SEQUENCE LISTING (8 sequences)

SEQ ID NO: 1 - DNA, 28 bp, synthetic construct
  source 1..28: mol_type = other DNA
actagcagctactgactagagagtgcag

SEQ ID NO: 2 - DNA, 28 bp, synthetic construct
  source 1..28: mol_type = other DNA
agtctgcagatagtagctgctgcagctg

SEQ ID NO: 3 - DNA, 29 bp, synthetic construct
  source 1..29: mol_type = other DNA
agcagtatgctagtgcgctactactatag

SEQ ID NO: 4 - DNA, 15 bp, synthetic construct
  source 1..15: mol_type = other DNA
tcgtctgtaaccatt

SEQ ID NO: 5 - DNA, 13 bp, synthetic construct
  source 1..13: mol_type = other DNA
tcgtctgtaacca

SEQ ID NO: 6 - DNA, 15 bp, synthetic construct
  source 1..15: mol_type = other DNA
atgacgtacttacgc

SEQ ID NO: 7 - DNA, 15 bp, synthetic construct
  source 1..15: mol_type = other DNA
ttgacacttagtacc

SEQ ID NO: 8 - DNA, 15 bp, synthetic construct
  source 1..15: mol_type = other DNA
ggattcgattagtag

(19) United States
(12) Patent Application Publication
(10) Pub. No.: US 2024/0309450 A1
(43) Pub. Date: Sep. 19, 2024

(54) ENCODED NUCLEIC ACID METHYLATION ASSAYS

(71) Applicant: PLENO, INC., San Diego, CA (US)

(72) Inventors: Jeffrey BRODIN, Poway, CA (US); Lorenzo BERTI, Poway, CA (US); Donald Brian EIDSON, San Diego, CA (US); Christian SCHLEGEL, San Diego, CA (US); Angela BLUM, San Diego, CA (US); Rachel SCHOWALTER, San Diego, CA (US); Ludovic VINCENT, San Diego, CA (US); Allen ECKHARDT, San Diego, CA (US); Pieter VAN ROOYEN, San Diego, CA (US); Gavin STONE, San Diego, CA (US)

(21) Appl. No.: 18/670,329

(22) Filed: May 21, 2024

Related U.S. Application Data
(63) Continuation of application No. PCT/US2022/037778, filed on Jul. 21, 2022.
Application No. PCT/US2022/037778 is a continuation of application No. PCT/US2021/060647, filed on Nov. 23, 2021.
(60) Provisional application No. 63/314,384, filed on Feb. 26, 2022.
(60) Provisional application No. 63/346,052, filed on May 26, 2022.

(51) Int. Cl. C12Q 1/6876; C12Q 1/682; C12Q 1/6844

(52) U.S. Cl. CPC C12Q 1/6876; C12Q 1/682; C12Q 1/6846; C12Q 2600/154

(57) ABSTRACT

Methods are provided for conducting an assay for a set of targets. The methods include (a) performing a methylation sensitive transformation process on a sample comprising a set of target nucleic acid sequences; (b) performing a hybridization and ligation reaction with an encoded detection probe from a set of encoded detection probes unique for each of the targets, the probes comprising a nucleotide sequence code from a set of nucleotide sequence codes to generate an amplifiable probe, wherein the probes unique for unmethylated targets are not amplifiable; and (c) performing an amplification and detection process on the sample, wherein the methylated targets are detected by decoding the amplified codes associated with the methylated targets.

### Related Applications

This application is a continuation application of International Application No. PCT/US2022/037778, filed Jul. 21, 2022, which claims the benefit of U.S. Provisional Application No. 63/346,052, filed on May 26, 2022; U.S. Provisional Application No. 63/314,384, filed on Feb. 26, 2022; and International Patent Application No. PCT/US2021/060647, filed on Nov. 23, 2021, each of which is herein incorporated by reference in its entirety.

### Incorporation by Reference of Sequence Listing

The present application is being filed along with a Sequence Listing in electronic format. The Sequence Listing is provided as a file entitled 64100-715_301_SL.XML, created May 21, 2024, which is 7.83 kilobytes in size. The information in the electronic format of the Sequence Listing is incorporated by reference in its entirety.

### Field of the Invention

The invention relates to encoded nucleic acid methylation assays, in which a target analyte is detected based on association of the target with a code, and detection of the code as a surrogate for detection of the target analyte.

### BACKGROUND OF THE INVENTION

Many assays such as single base detection assays require high-level of sensitivity and specificity and are associated with low signal level. Low signal requires amplification (e.g., PCR, immunostaining cascades, and the like) resulting in complex and lengthy protocols, high-level of background and other biases limiting the performance of the assay. There is a need in the art for assays that are easier to read and detect at higher sensitivity than the analyte itself.

### BRIEF DESCRIPTION OF DRAWINGS

The features and advantages of the present invention will be more clearly understood from the following description taken in conjunction with the accompanying drawings, which are not necessarily drawn to scale, and wherein:

FIG. 1 is a diagram illustrating an encoding method that uses a 4-state encoding trellis with 3 transitions per state.

FIG. 2 is a diagram illustrating an encoding trellis for a 4-bases-per-cycle pyrosequencing.

FIG. 3 is a diagram illustrating a pyro-code example, followed by a snapshot from a spreadsheet with relevant parameters. FIG. 3 discloses SEQ ID NOS 1-3, respectively, in order of appearance.

FIG. 4 shows a hypothetical emission spectrum, which is detected at varying intensities by Channel A, Channel C and Channel G, and not detected by Channel T.

FIG. 5 is a schematic diagram of an example of a coded padlock probe.

FIG. 6A is a schematic diagram illustrating an example of adding an index sequence to a probe using a ligand protein coupling strategy.

FIG. 6B is a schematic diagram illustrating an example of adding an index sequence to a probe by restriction endonuclease cleavage followed by index ligation.

FIG. 7 is a schematic diagram of an example of a surface-based workflow, wherein the target is immobilized on the surface and used to template the ligation of a probe to produce a circular modified probe.

FIG. 8 is a schematic diagram of an example of a surface-based workflow, wherein the target immobilized on the surface is used as a primer to initiate the amplification of the probe to generate a nanoball product.

FIG. 9 is a schematic diagram of an example of a surface-based workflow, wherein the probe is immobilized on the surface and the immobilized probe is used capture a target.

FIG. 10A is a schematic diagram of an example of a dual probe workflow, wherein a first probe is immobilized on the surface and the second probe is in solution.

FIG. 10B is a schematic diagram illustrating an example of a surface bound probe that includes a second surface adapter.

FIG. 11 is a schematic diagram illustrating an example of a process for synthesizing a surface bound probe using a splint oligonucleotide.

FIG. 12A is a schematic diagram illustrating an example of a surface bound probe structure comprising a second surface adapter.

FIG. 12B is a schematic diagram illustrating an example of a surface bound probe structure comprising a second surface adapter and showing a process of performing bridge amplification.

FIG. 13 is a schematic diagram illustrating an example of a probe that includes a restriction enzyme site that may be used to linearize the probe for capture on a flow cell for bridge amplification prior to sequencing.

FIG. 14A is a schematic diagram illustrating an example of a process of using a surface-bound oligonucleotide to initiate an RCA reaction.

FIG. 14B is a schematic diagram illustrating an example of capturing a nanoball on a cation-coated surface.

FIG. 14C is a schematic diagram illustrating an example of capturing a nanoball on a streptavidin-coated surface.

FIG. 14D is a schematic diagram showing an example of using a biotin-streptavidin linkage to perform a surface-bound RCA reaction.

FIG. 15A is a schematic diagram of a transformation process for circularizing a linear probe to form a circular modified probe.

FIG. 15B is a schematic diagram showing RCA amplification of the circular modified probe to yield a nanoball product.

FIG. 15C is a schematic diagram showing the addition of sequencing adapters to a nanoball concatemer for subsequent clustering and sequencing.

FIG. 16A is a schematic diagram of an example of a portion of the nanoball of FIG. 15 that includes restriction sites that may be used to separate repeated copies of the probe in the nanoball.

FIG. 16B is a schematic diagram of an example of a portion of the nanoball including restriction sites of FIG. 16A.

FIG. 17 is a schematic diagram of an example of a process for circularizing and amplifying unit length nanoball fragments to produce multiple RCA nanoball products.

FIG. 18 is a schematic diagram of an example of an alternative process for circularizing and amplifying unit length nanoball fragments to produce multiple RCA nanoball products.

FIG. 19 is a schematic diagram of an example of a process for capturing an unknown region of a target for sequencing.

FIG. 20 is a flow diagram of an example of a methylation assay workflow.

FIG. 21 is a flow diagram of an example of a methylation assay workflow of direct detection of methylated nucleic acids using methylation-specific inhibition of ligation.

FIG. 22 is a schematic diagram illustrating a process for detecting methylated nucleic acids using padlock probe ligation in combination with methylation-specific restriction enzyme digestion.

FIG. 23 is a schematic diagram illustrating an alternative embodiment of the process described with reference to FIG. 22.

FIG. 24 is a schematic diagram illustrating a process of using dual hybridization probes in combination with a methylation sensitive endonuclease digestion reaction to differentiate a methylated target sequence and an unmethylated target sequence.

FIG. 25 is a schematic diagram illustrating an example of a solution-phase proximity ligation process that uses a linear detection probe in combination with a modified methyl binding protein for detection of methylated cytosine.

FIG. 26 is a schematic diagram illustrating an example of a solid-phase detection process for detection of a methylated site in a target sequence of interest.

FIG. 27 is a schematic diagram illustrating some of the factors considered in the design of an encoded probe for decoding by hybridization.

FIG. 28A is a schematic diagram illustrating an overview of process for decoding by hybridization.

FIG. 28B is a schematic diagram illustrating the code space in decoding by hybridization.

FIG. 29 is a schematic diagram of an example of a method for encoding symbols onto each segment of a code.

FIG. 30 is a schematic diagram of another example of a method for encoding symbols onto a code wherein the length of the code sequence comprises a single segment that requires a relatively large number of decoding oligos.

FIG. 31 is a schematic diagram of another example of a method for encoding symbols onto a code wherein a mix of segment number and flows/segment in the decoding process balances the length of a code and the complexity required in the decoding oligo pool.

FIG. 32 is a screenshot of an example of the permutations (e.g., colors, flows/segment, total segments, and total flows) that may be used to achieve a relatively large combination space (codespace) from which to select a subset of codes.

FIG. 33A is a plot showing the relationship of the number of codes in a code space.

FIG. 33B is a summary table of the number of segments, flows, and colors required for a given number of targets for detection.

FIG. 34 is a schematic diagram of an example of a trellis code and a process of using the trellis code to select a set of codes with desired properties for an assay from a large code space.

FIG. 35A is a representation of a strategy for designing oligo segments on a probe that will encode for the symbols that make up the trellis code (or other type of code).

FIG. 35B shows examples of excluded sequences and temperature parameters for the strategy for designing oligo segments on a probe of FIG. 35A.

FIG. 36 is a representation of an overview of a decoding process comparing hard decoding vs. soft decoding. FIG. 36 discloses SEQ ID NOS 4-8, respectively, in order of appearance.

FIG. 37 is a schematic diagram of an example of a soft decoding process that may be used in the assays of the invention.

FIG. 38 is a summary of a channel model for a base calling algorithm that may be used in a soft decoding process.

FIG. 39 is a flow chart illustrating aspects of the disclosed methods.

FIG. 40A is a plot showing qPCR amplification plot after 30 minutes of cutting with Hha I.

FIG. 40B is a plot showing qPCR amplification plot after 2 hours of cutting with Hha I.

FIG. 41 is a schematic diagram showing an example of a code element probe and some of the workflow steps in the multiplexed assay.

FIG. 42 is a schematic diagram and a plot showing the specificity of target interaction in the multiplexed assay.

FIG. 43 is a schematic diagram and a plot showing the allele ratio in the multiplexed assay.

FIG. 44A is a photo showing the density, size and uniformity of nanoballs generated in an RCA reaction performed on a polylysine-coated MiSeq flow cell.

FIG. 44B is a photo showing the density, size and uniformity of nanoballs generated in an RCA reaction performed on a polylysine-coated microplate.

FIG. 45A is a panel of photos of a comparison of nanoballs generated on a polylysine surface to nanoballs absorbed to a surface after an RCA solution reaction.

FIG. 45B is a pair of plots of a comparison of nanoballs generated on a polylysine surface to nanoballs absorbed to a surface after an RCA solution reaction.

### SUMMARY OF THE INVENTION

In one embodiment of the invention, a method is provided of conducting an assay for a set of targets, the method comprising: (a) dividing a sample comprising a set of target nucleic acid sequences into two parallel samples for analysis, one for methylation specific analysis (MS sample) and one for non-methylation specific analysis (nMS sample); (b) adding a methylation specific binding moiety to the MS sample and incubating for a period of time sufficient for binding to methylated cytosines; (c) hybridizing an encoded detection probe in a set of encoded detection probes to each target in the set, the probes comprising a nucleotide sequence code from a set of nucleotide sequence codes; (d) performing a molecular transformation in which a modified probe is produced to enable differentiation of a methylated and an unmethylated status of each target in the MS sample; and (e) amplifying and detecting the codes bound to target in the nMS sample and the codes bound to the targets that are unmethylated in the MS sample, wherein the targets are detected by decoding the amplified codes associated with the targets.

In another embodiment, a method is provided of conducting an assay for a set of targets, the method comprising: (a) performing a hybridization and ligation reaction on a sample comprising a set of target nucleic acid sequences in which each target is hybridized to an encoded detection probe in a set of encoded detection probes, the probes comprising: (i) a nucleotide sequence code from a set of nucleotide sequence codes and (ii) a sequence for a methylation sensitive endonuclease site, wherein the bound probes are circularized; (b) performing a transformation process on the sample, in which the methylation sensitive endonuclease cuts the targets that are unmethylated and does not cut the methylated targets; and (c) performing an amplification and detection process on the sample, in which the circularized probes bound to methylated targets are amplified and the cut probes bound to unmethylated targets are not amplified, wherein the methylated targets are detected by decoding the amplified codes associated with the methylated targets.

In other aspects of the invention, a method is provided of conducting an assay for a set of targets, the method comprising: (a) performing a transformation process on a sample comprising a set of target nucleic acid sequences, in which a methylation sensitive endonuclease cuts the targets that are unmethylated at a target site but does not cut the methylated targets; (b) performing a hybridization and ligation reaction with an encoded detection probe, from a set of encoded detection probes unique for each of the targets, the probes comprising: (i) a nucleotide sequence code from a set of nucleotide sequence codes and (ii) a sequence for the methylation sensitive endonuclease site, wherein the probes hybridized to methylated targets are circularized thereby generating an amplifiable probe, and wherein the probes unique for unmethylated targets are not circularized and are not amplifiable; and (c) performing an amplification and detection process on the sample, wherein the methylated targets are detected by decoding the amplified codes associated with the methylated targets.

In one instance, a method is provided of conducting an assay for a set of targets, the method comprising: (a) performing a hybridization and ligation reaction on a sample comprising a set of target nucleic acid sequences in which a pair of encoded ligation detection probes, from a set of encoded ligation detection probes is hybridized to each of the targets, each pair of probes comprising: (i) a nucleotide sequence code from a set of nucleotide sequence codes and (ii) a sequence for a methylation sensitive endonuclease, wherein the probes are hybridized to the target immediately adjacent to each other leaving no gap and are ligated to produce a ligated dual probe strand; (b) performing a methylation sensitive endonuclease reaction on the sample in which the hybridized targets that are unmethylated are cut and the hybridized targets that are methylated are not cut; (c) performing a single-stranded DNA ligation reaction on the sample in which uncut methylated target is circularized to generate an intact primer site, and in which cut unmethylated target is circularized to generate two circular elements that do not include an intact primer site; and (d) performing an amplification and detection process on the sample, wherein the methylated targets are detected by decoding the amplified codes associated with the methylated targets. In another instance, steps (a) and (b) are reversed.

The methods of the invention include a method of conducting an assay for a set of targets, the method comprising: (a) performing a hybridization and methyl binding moiety reaction on a sample comprising a set of target nucleic acid sequences, in which: (i) a location-specific oligonucleotide probe is hybridized adjacent to a site on each of the targets, the location-specific oligonucleotide probe comprising a first hybridization sequence complementary to the target and a second hybridization sequence complementary to a corresponding sequence on a linear encoded detection probe in a set of linear encoded detection probes, (ii) a methylation specific binding moiety is bound to a methyl group on each of the targets that are methylated at the site, the moiety modified with an oligonucleotide complementary to a corresponding sequence on the detection probe, and (iii) the detection probe is hybridized to the sequence on the location specific oligonucleotide probe and the oligonucleotide on the methyl binding moiety which effectively circularizes the detection probe, the detection probe comprising a nucleotide sequence code from a set of nucleotide sequence codes; (b) performing a ligation reaction on the sample, in which the detection probe is circularized to generate an intact primer site for the targets that are methylated at the site, but not for the targets that are not methylated at the site; and (c) performing an amplification and detection process on the sample, wherein the methylated targets are detected by decoding the amplified codes associated with the methylated targets.

In another embodiment, a method is provided of conducting an assay for a set of targets, the method comprising: (a) contacting a sample comprising a set of target nucleic acid sequences with a substrate having a methylation specific binding moiety immobilized thereto, wherein targets having one or more methylated cytosines are captured on the substrate; (b) hybridizing a set of target-specific, encoded detection probes to the fully or partially methylated targets in the set, the probes comprising: (i) a nucleotide sequence code from a set of nucleotide sequence codes and (ii) an amplification primer binding site; (c) optionally, performing a ligation reaction in which the detection probe is circularized; and (d) performing an amplification and detection process on the sample, wherein the methylated targets are detected by decoding the amplified codes associated with the methylated targets.

In the methods of the invention for conducting an assay for a set of targets, each code may include at least one segment encoding one or more symbols that correspond to a sequence of one or more nucleotides.

In the methods of the invention, the set of encoded detection probes may comprise at least 10, 100, 1,000, or 10,000 encoded detection probes and each of the encoded detection probes may comprise a soft decodable code.

In various embodiments, the codes are decoded using a soft decision decoding method. For example, decoding the codes may include recording signal produced in response to interrogation of each segment of the codes and, upon completion of the interrogation, determining a probably of the presence of each of the codes by applying a soft-decision probabilistic decoding algorithm to the recorded signal. The signal produced may include, but is not limited to, signal from one or a combination of nanopore sequencing, next-generation sequencing, massively parallel sequencing, Sanger sequencing, sequencing by synthesis (SBS), pyrosequencing, sequencing by hybridization, decoding by hybridization, single molecule real-time sequencing, SOLiD, and sequencing by ligation.

In one embodiment, interrogation of the code segments including one symbol corresponding to more than one nucleotide is performed by decoding by hybridization.

In some instances, at least one of the code segments is interrogated more than one time by hybridization with one or more hybridization probes each having at least one label to produce the signal. At least four different labels may be utilized in the decoding by hybridization. In one example, each code includes at least four segments and at least sixteen symbols.

In the case that at least one of the code segments is interrogated more than one time by hybridization with one or more hybridization probes each having at least one label to produce the signal, a unique number of possibilities at each of the code segments includes up to a number of the different labels to the power of a number of the hybridizations per segment.

The label on the hybridization probe may be an optical label. The label may be a fluorescent label. In some instances, at least one hybridization probe may include two or more of the labels to create a pseudo label and generate a larger number of the code symbols.

In the methods of the invention, the set of targets may include tens of target analytes, hundreds of target analytes, thousands of target analytes, or tens of thousands of target analytes.

In various embodiments, encoded probes, sets of encoded probes, and compositions including the sets of encoded probes are provided.

In one instance, a set of coded oligonucleotide probes is provided, each probe including a code from a set of codes. In this instance, each code is a soft decodable code that includes at least one segment encoding one or more symbols that correspond to a sequence of one or more nucleotides.

The set of coded oligonucleotide probes may include padlock probes.

The set of coded oligonucleotide probes may include at least 10, 100, 1,000, or 10,000 probes.

### DETAILED DESCRIPTION OF THE INVENTION

### Terminology

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” “comprising,” “include,” “includes,” and “including,” are intended to be non-limiting, such that recitation of items in a list is not to the exclusion of other like items that may be substituted or added to the listed items.

“Preferably,” “commonly,” and “typically” and the like are not utilized herein to limit the scope of the claimed embodiments or to imply that certain features are critical or essential to the structure or function of the claimed embodiments. These terms are intended to highlight alternative or additional features that may or may not be utilized in a particular embodiment of the present disclosure.

“Substantially” is utilized herein to represent the inherent degree of uncertainty that may be attributed to any quantitative comparison, value, measurement, or other representation and to represent the degree by which a quantitative representation may vary from a stated reference without resulting in a change in the basic function of the subject matter at issue.

“Target” means a nucleic acid analyte (e.g., mRNA, cfDNA etc.) or a proxy for the target analyte of interest (e.g., an antibody conjugated with oligonucleotide). Thus, in some instances, the term “target” and the term “target analyte” are used interchangeably. “Target” with respect to a nucleic acid may include the presence or absence of one or more methyl groups on the nucleic acid target.

“Coded” and “encoded” are intended to have the same meaning and are herein used interchangeably.

“Decoding” with respect to a code includes determining the presence of a known code or a probability of the presence of a known code with or without determining the sequence of the code. Decoding may be hard decision decoding. Decoding may be soft decision decoding.

“Hard decision decoding” or “hard decision” refers to a method or model that includes making a call for each nucleotide in a nucleic acid segment (commonly referred to as a “base call”) in order to determine the sequence of nucleotides in the nucleic acid segment. Models of the invention incorporate hard decision decoding models. The particular nucleic acid being decoded may be or include a code of the invention.

“Soft decision decoding” or “soft decision” refers to a method or a model that uses data collected during a sequencing or decoding process to calculate a probability that a particular nucleic acid or nucleic acid segment is present. The probability may optionally be calculated without making a base call for each nucleotide in a nucleic acid segment. In another example, a probability is calculated without making a hard call that a string of nucleic acids in a segment are present. Instead of making a hard call for each nucleotide or nucleotide segment, a probabilistic decoding algorithm is applied to the recorded signal upon completion of signal collection. A probability of the presence of each of the codes may be determined without discarding signal in contrast to hard decision decoding method in which hard calls are made during the signal collection process. In soft decision decoding, the data may, for example, include or be calculated from, intensity readings in spectral bands for signals produced by the sequencing/decoding chemistry. In one embodiment, soft decision decoding uses data collected during a sequencing/decoding process to calculate a probability that a particular nucleic acid segment from a known set of sequences is present. Models of the invention may be used for soft decision decoding. The particular nucleic acid or nucleic acid segment being decoded may be or include a code of the invention.

“Phasing” or “signal phasing” means misalignment of SBS cycles during an SBS process caused by the non-incorporation of a nucleotide during a cycle or by the incorporation of two or more nucleotides during an SBS cycle.

“Droop” or “signal droop” means signal decay that occurs during an SBS process, which may be caused by some complementary strands being synthesized as part of the SBS process being blocked, preventing further nucleotide incorporation.

“Sample” means a set of nucleic acids for testing. A sample preparation process may be used to produce a sequencing-ready sample from a raw sample or partially processed sample. Note that one or more samples may be combined for sample preparation and/or sequencing and may be distinguished post-sequencing using sample-specific DNA barcodes linked to sample fragments. A sample may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes.

“Set” includes sets of one or more elements or objects. A “subset” of a set includes any number elements or objects from the set, from one up to all of the elements of the set.

“Crosstalk” refers to the situation in which a signal from one nucleotide addition reaction may be picked up by multiple channels (referred to as “color crosstalk”) or the situation in which a signal from a nanoball or sequencing cluster interferes with an adjacent or nearby cluster or nanoball (referred to as “cluster crosstalk” or “nanoball crosstalk”).

“Color channel” means a set of optical elements for sensing and recording an electromagnetic signal from a sequencing reaction. Examples of optical elements include lenses, filters, mirrors, and cameras.

“Spectral band” or “spectral region” means a continuous wavelength range in the electromagnetic spectrum.

“Invention,” “the invention” and the like are intended to refer to various embodiments or aspects of subject matter disclosed herein and are not intended to limit the invention to the specific embodiments or aspects of the invention referred to.

“Identify,” “determine” and the like with respect to codes, targets or anlaytes of the invention are intended to include any or all of: (A) an indication of the presence or absence of the relevant code, target or anlayte, (B) an indication of the probability of the presence or absence of the relevant code, target or anlayte, and/or (C) quantification of the relevant code, target or anlayte.

The description and examples should not be construed as limiting the scope of the invention to the embodiments and examples described herein, but as encompassing all modifications and alternatives falling within the true scope and spirit of the invention.

### Encoded Nucleic Acid Methylation Assays

The invention provides encoded nucleic acid methylation assays. At a high level, in an encoded nucleic acid methylation assay, a target analyte (“target”) is detected based on association of the target with a code and decoding of the code is a surrogate for detection of the analyte.

In various embodiments, an encoded nucleic acid methylation assay may include a recognition event in which a target is uniquely recognized by a recognition element. The recognition event may be effected by submitting targets of a set of targets to a recognition event, in which each target is uniquely recognized by and bound to a recognition element associated with a code, thereby yielding a set of coded targets comprising the target and the recognition element.

In various embodiments, an encoded nucleic acid methylation assay may include a transformation event, in which a high-fidelity molecular transformation of the recognition element associated with a code produces a modified recognition element. The transformation event may be effected by submitting each recognition element of the set of coded targets to a transformation event, in which a molecular transformation of each recognition element produces a modified recognition element, thereby yielding a set of modified recognition elements comprising the code.

In various embodiments, an encoded assay may include a detection event, which decodes the code as a surrogate for detection of the analyte, e.g., by recognizing or determining the presence or the sequence of the code (and optionally other elements). The detection event may include an amplification step in which each code of the set of modified recognition elements is amplified, thereby yielding a set of amplified codes. Amplified codes of the set of amplified codes may have their sequences determined using a variety of techniques, including for example, microarray detection, or nucleic acid sequencing. In some cases, the decoding step may be integrated with the amplification step, e.g., as in amplification with intercalating dyes.

In one embodiment, the method may include:
(i) submitting each target of a set of targets to a recognition event, in which each target is uniquely recognized by and bound to a recognition element associated with a code, thereby yielding a set of coded targets comprising the target and the recognition element;
(ii) submitting each recognition element of the set of coded targets to a transformation event, in which a molecular transformation of each recognition element produces a modified recognition element, thereby yielding a set of modified recognition elements comprising the code;
(iii) submitting each code of the set of modified recognition elements to an amplifying event, in which each code is amplified, thereby yielding a set of amplified codes;
(iv) submitting each amplified code of the set of amplified codes to a detection event, thereby determining the presence of the code or the nucleic acid sequence of the code.

In one embodiment, the method may include:
(i) a recognition event in which the target is uniquely recognized by a recognition element, which associates a code (and optionally other elements) with the target via the recognition element;
(ii) a transformation event, in which a high-fidelity molecular transformation of the recognition element produces a modified recognition element that produces a readable code;
(iii) a detection event, which detects the code as a surrogate for detection of the analyte, e.g., by recognizing or determining the presence or the sequence of the code (and optionally other elements).

As described in more detail herein, the recognition event, transformation event, and the decoding event may occur sequentially, or combinations of the steps may occur simultaneously, e.g., as a single combined step. For example, the transformation event and the coding event may be simultaneous, such that the sequential process involves (i) recognition event, followed by (ii) transformation event/coding event, followed by (iii) detection event.

To further illustrate the encoded assays:
(i) In the recognition event, the target may be detected by a targeted molecular binding event, such as binding of the target by a complementary sequence or a polypeptide binder.
(ii) In the transformation event, a ligation or a gap-fill ligation may produce the modified recognition element, i.e., a version of the recognition element that is ligated or gap-fill ligated.
(iii) In the coding event, a code reagent may be associated with the modified recognition element based on recognition of the modified recognition element. For example, the novel coded padlock probes of the invention may be configured with a sequence that recognizes the modified recognition element and circularize only if the modified recognition element is present.
(iv) In the detection event, the reading of the code may involve any means of determining the presence of or the sequence of the code (and optionally other elements).

The codes may be error corrected and thus easy to distinguish from each other, so they can be detected a low abundance and in the presence of high level of background and in the presence of many other codes.

Since many assays can be converted into codes, the invention provides for multi-omic assays where a sample is analyzed in multiple parallel workflows that are analyte-dependent and then converge codes that can then be detected simultaneously in a single platform. Parallel assay workflows may be merged into a single workflow, where multiple targets and target-types (e.g., nucleic acids and polypeptides) may be detected simultaneously in a single workflow and also read simultaneously within the same readout platform.

Following recognition and transformation, the codes may be decoded and matched to targets for identification and/or quantification of targets present in the sample.

### Code Design and Decode

The encoded assays of the invention make use of codewords or codes. The codes may be detected as surrogates in the place of direct analysis of target analytes. As an example, a target analyte may be a particular nucleic acid fragment (e.g., a nucleic acid fragment with a specific mutation); in the assays of the invention, a codeword may be associated with the nucleic acid fragment and the codeword may be decoded to identify the presence of and/or quantify the nucleic acid fragment in the sample.

For example, a code may in some embodiments be a predetermined sequence ranging from about 3 to about 100 nucleotides or about 3 to about 75 nucleotides. Codes may have sequences selected to avoid inadvertent interaction with other assay components, such as targets, probes, or primers. Code sequences may be selected to ensure that codes differ from each other to permit unique identifiability during the decoding process.

The invention includes a dataset or database of codes generated using the methods of the invention. The dataset or database may associate the codes with other assay elements, such as primers or probes linked to the codes. The invention also includes a method of making a probe set comprising synthesizing probes having the sequences set forth in the dataset or database.

### Homopolymer-Free Encoding

In one embodiment, the codes are homopolymer-free codes. For standard genomic applications that use a full 4-ary nucleotide alphabet of {ACGT}, the method uses a 4-state encoding trellis with 3 transitions per state.

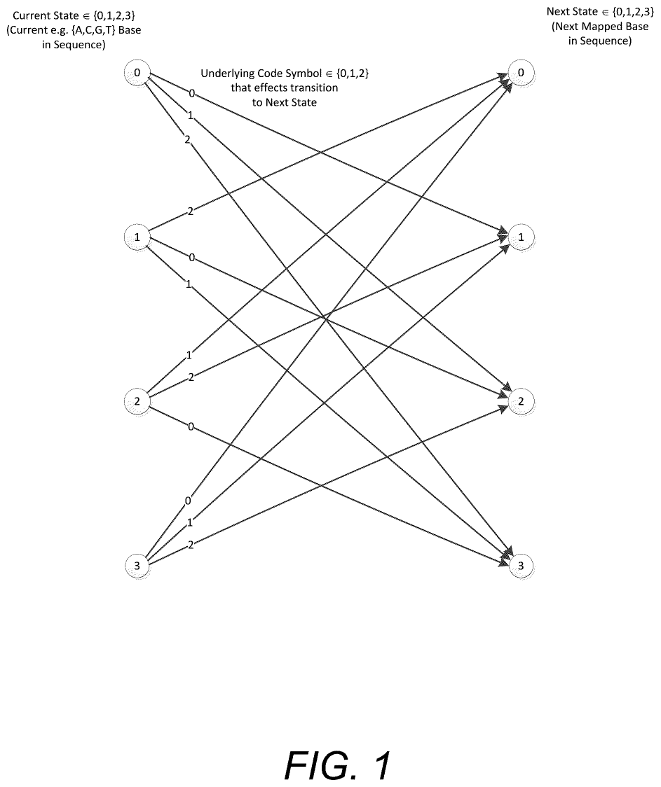

As illustrated in FIG. 1, the current state is the last mapped nucleotide, and the next state is the next (to-be) mapped nucleotide. By forbidding a transition from the current state (say, the ‘A’ state) in the present trellis section (of 4 states), to the analogous same state (of ‘A’) in the next trellis section (of 4 states), a repeated mapping to the same nucleotide base [symbol] in any generated sequence [symbol] is avoided. An ‘A’ state can only transition to a ‘C’, ‘G’, or ‘T’ state in the next trellis section. Since this involves 3 transitions per state, the mapping trellis is mated to an underlying 3-ary (i.e., ternary-) alphabet error correction code that drives transitions through trellis sections. The underlying (ternary) error correction code is the mechanism that guarantees all generated codewords differ in multiple sequence positions.

A similar method may apply to 3-ary alphabets (where only 3 of the four nucleotide bases, say {CGT} are used), and 5-ary or higher alphabets, where the underlying correction code uses an alphabet of order one less than the mapping alphabet.

In one embodiment, codes for the set of codes are selected using a 4-ary alphabet, avoid homopolymers, and every code in the set is different from every other code in the set. The codes may be generated using the trellis method.

In one embodiment, codes for the set of codes are selected using a 3-ary alphabet, avoid homopolymers, and every code in the set is different from every other code in the set. The codes may be generated using the trellis method.
(i) In another embodiment, a homopolymer-free code composed from a 4-ary nucleotide alphabet of {ACGT} may be generated as follows:
(ii) From GF(4) (i.e., the quaternary algebraic alphabet), select an error correction code that will deliver many more codewords than necessary (because some of the generated codewords will later be eliminated);
(iii) Generate all of the codewords for the code;
(iv) Assess the number of repeated symbol locations in each codeword;
(v) Re-order the list of codewords, sorting by the number of base-repeat instances in each codeword.
(vi) From the re-ordered sort, keep only the top K codewords, where K is the desired library size of codewords (this will eliminate the codes with the highest number of polymer-repeats; each repeat will require subsequent fixing that weakens the overall code.)
(vii) For each codeword in the list of survivors, ‘smart fix’ the repeat positions in each codeword with the following procedure:
   a. Start from the beginning base position in a codeword, and find the first repeat instance of a base;
   b. Go to the second base in the first repeat instance, its base assignment will require change;
   c. If the second base is not at the end of a codeword, look ahead one base position in the codeword, and assess the assignment there;
   d. For the second base (in the repeat), choose a new base assignment that is also different from the base assigned one sample ahead; n that, in addition to removing a length-2 run, this step will also fix a length-3 run;
   e. Process the revised codeword at each remaining repeat location, fixing the second base in each repeat using the process outlined in steps c-d.

This method will eliminate all repeats. The same method can be applied to generate homopolymer free codes for 3-ary alphabets (eg., {C, G, T}), and larger 5-ary+ alphabets (such as oligopolymers).

Codes optimized for pyrosequencing and similar cyclic serial dispensation schemes

The invention provides a locus code-encoding approach for pyrosequencing or similar serial (rather than pooled) primer dispensation methods. The method generates homopolymer-free codes.

When the locus code is encapsulated between header and tail bases, all generated codewords finish decoding at the same time. The technique avoids unexpected spurious incorporations that change how long in time that a codeword needs to finish its decoding. This is important because then a sequencer only need sample for a prescribed number of samples to obtain complete data for decoding the samples, regardless of the underlying codeword. This also keeps all codewords candidates aligned, so that the theoretical design distances between codewords are maintained.

The aforesaid synchrony ensures that soft decision block decoding techniques can be applied during the decoding of its blocks of samples. This soft decision decoding guarantees that signal to noise ratio (SNR) requirements are improved by at least 2 dB [symbol] and sometimes by many factors-more when the signal strength significantly fades during the reception of codeword samples.

In pyrosequencing, nucleotides are dispensed sequentially (and non-overlappingly) in a cycle, such as G, C, T, A, G, C, T, A, G, C, . . . etc. This encoding is quite original because it doesn't directly encode bases; instead, it encodes base POSITIONs within G, C, T, A cycles. Each cycle element can be either populated, or unpopulated [symbol] and multiple elements within a cycle can be populated. For this to be implemented, the underlying code must be derived from a binary alphabet, with 1s and 0s. To emphasize, with these codes, more than one base can be incorporated within a single G, C, T, A dispensation cycle. This also implies that sequencing, though serial in nature, can be fast. And with the underlying {0,1} alphabet that underpins and drives the encoding of the populated/unpopulated cycle positions, all codewords are guaranteed to be of the same length [symbol] and to finish decoding in the same amount of time.

To provide coding gain, the sequence of 0s and 1s that compose each codeword are derived from constructions of optimal binary error correction codes. Such codes possess many redundant parity bits, and these parity bits are designed such that each codeword varies from each other in multiple positions. This quality results in strong error correction capabilities.

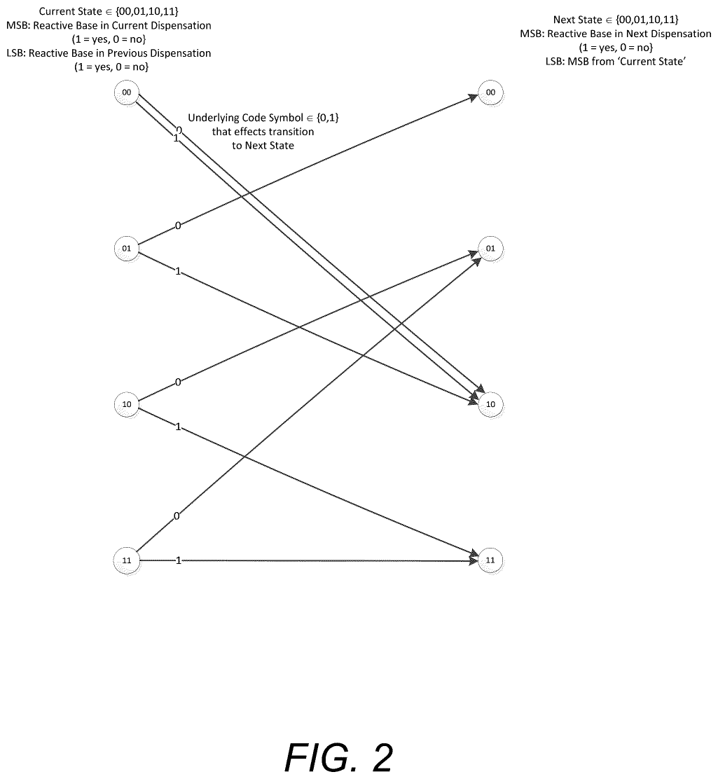

FIG. 2 illustrates an encoding trellis for a 4-bases-per-cycle pyrosequencing. The techniques may be used for encoding 3-cycle, 3-base-alphabet, and 5+-cycle, 5-and-higher-alphabet oligo-polymer hybrid schemes.

Note the use of 4 states in the trellis. Each state represents previous mappings of that last two positions:
(i) both unpopulated, (00);
(ii) both populated, (11);
(iii) newest-populated and older-unpopulated, (10);
(iv) newest-unpopulated and the older populated, (01).

Transitions to next states indicate an update which either does not populate or does populate the next position in a sequence.

Four (4) states are used to correctly implement a pyrosequencing scheme that is homopolymer-free; one position is populated every 3 positions. Note that if 3 consecutive positions were allowed to be unfilled, then the 4^{th} position would need to be filled (because an unzipped hybrid will have an opening to at least one of the four nucleotides). That 4^{th} position being filled would result in generation of a homopolymer (repeat) of bases in a sequence [symbol] since the last filled base was the same base in the cycle before.

This aforementioned restriction explains the double transition from the 00 state to the 10 state in the trellis diagram. A current state of 00 transitioning to a next state of 00 would imply 3 positions in a row were unfilled.

Optimal error correction codes are constructed to maximize distance between their sets of codewords. They are not constrained to disallow runs of three consecutive zeros. That would reduce the degrees of freedom they use to maximize distance. By contrast, the mappings to pyro-sequenced positions comply with homopolymer-free and pyrosequencing constraints.

All other transitions in the picture design trellis are natural results of populating a position with a ‘0’ or a ‘1’ and updating the next state to reflect that transition. Since 7 of the 8 transitions in the trellis perfectly express the underlying error correction code's structure, such a code can be quite effective and powerful.

Weakening transitions occur when the underlying code has 3 consecutive zeros. One way to reduce those appearances is to use the sorting methodology described above. This method modestly reduces the library of codes. This method also ensures that the pyro-mapped codewords that best reflect the underlying binary code's structure are faithfully reproduced, while those least reflective are not.

Another method to improve the weakening due to transitions involves breaking up strings of zeros by interleaving the code. Within a code, the (systematic) information section of bits [symbol] which precede the redundant section of parity bits [symbol] are the bits where the most consecutive zeros are usually seen. One way to eliminate those strings of zeros is to interleave the entire code design, so that the parity and information bits are intermingled. All codewords may be intermingled by the same interleaving pattern. The interleaving technique does not help for the all-zeros codeword, which is generated by almost all linear codes. The all-zeros codeword can be excluded from the codeword set.

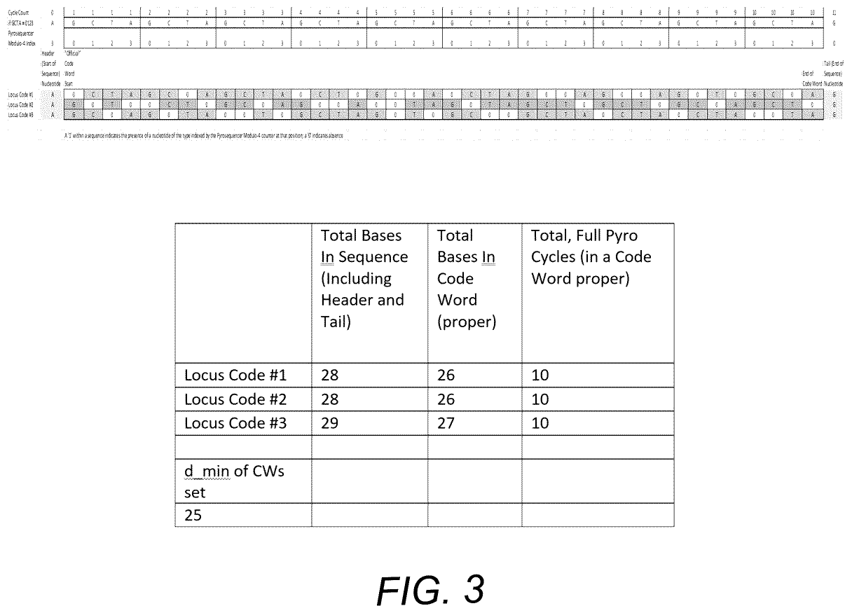

FIG. 3 shows a pyro-code example, followed by a snapshot from a spreadsheet with relevant parameters. The code is a 10-cycle, 40 position code that maps {GCTA} in cycles. It possesses a huge minimum distance between codewords, and is an example code accommodating three codewords. Note that the number of bases assigned to each codeword is not the same, although, clearly, from the illustration, all codewords are of the same time duration, and would finish decoding at the same time. Observe also the usage of populated ‘header’ and ‘tail’ positions. These are used to encapsulate the codeword and ensure that it is homopolymer free throughout. These terminating positions may be butted-up against the ends of the codewords for effective encapsulation.

### Amplifying and Reading Codes

In an encoded nucleic acid methylation assay, a target is detected based on association of the target with a code, and detection of the code is used as a surrogate for detection of the analyte. A variety of techniques may be used to amplify and read the codes. Examples include nanoballs, oligo clusters, oligo amplicons, bead-attached oligos, patterned oligos, and microarrays.

In one embodiment, codes of the invention are amplified using rolling circle amplification (RCA) to produce nanoballs that include many duplicates of the code. An RCA reaction may include one or more rounds of amplification to produce the nanoball product. A nanoball may be from about 10,000 to about 1,000,000 nucleotides in length. A nanoball may include from about 100 to about 10,000 copies of the amplified code.

In one embodiment, the codes of the invention are amplified using a linear PCR amplification reaction to generate double stranded DNA amplicon products.

In one embodiment, codes of the invention are amplified using bridge amplification to produce clusters of oligos on a surface.

In one embodiment, codes of the invention are amplified on bead surfaces to produce bead-attached oligos.

In one embodiment, the amplified codes are read in a sequencing reaction. Any sequencing technology may be used to sequence. Examples of sequencing technologies that may be used include sequencing by synthesis (e.g., pyrosequencing; sequencing by reversible terminator chemistry (Illumina)), avidity sequencing (Element Biosciences), sequencing by hybridization, sequencing by ligation, and nanopore sequencing.

In one embodiment, codes of the invention are detected using a patterned array, such as a microarray comprising oligos which are complimentary to the codes.

In one embodiment, the amplified codes are read using oligonucleotide probes in a hybridization-based reaction.

In one embodiment, codes of the invention are detected in situ, i.e., in a cell or tissue.

In one embodiment, in situ detection comprises reading the code in a sequencing reaction.

In one embodiment, codes of the invention are detected using an electronic/electrical sensing mechanism.

A variety of techniques and models may be used to decode a nucleic acid code of the invention. In one embodiment, the invention provides models that make use of hard decision decoding methods or models. In another embodiment, the invention provides models that make use of soft decision decoding methods or models.

When using soft decision decoding techniques, it is not necessary for the model to identify each base specifically. For example, signals generated during each nucleotide addition cycle of a sequencing process may be detected and recorded to produce a data set that may be used as input into a model of the invention to calculate a probability that a specific code is present without requiring a hard decoding model. Although it is not necessary in a soft decision decoding model to make a hard decision about the identity of each nucleotide, a model developed according to the methods of the invention may nevertheless include a model for assigning a probability or identity to each nucleotide in the sequence of a code.

Data gathered during a sequencing process may, for example, include intensity readings for signals produced by the sequencing chemistry in various spectral bands. For example, in some cases the data is collected across a set of spectral bands that corresponds to part or all of the spectral bands expected to be produced by a series of nucleotide extension steps during a sequencing process.

In some embodiments, it is not necessary to filter light from each nucleotide extension step in order to distinguish between the nucleotides. Instead, a set of intensity readings may be detected, stored and used as input into a model of the invention for determining a probability that a particular code is present. In other embodiments, one or more filters may be used to refine signals from a sequencing process.

A model may be developed or trained using sequencing data from known codes, such as signal intensity data across a predetermined spectrum, during a sequencing process. The model may be used to calculate a set of probabilities across a set of one or more codes, indicating, for example, for each code, a probability that it is present in a sample.

In some cases, the model is developed or trained using data corresponding to color intensity signals across multiple color channels. In some cases, the model is developed or trained using data corresponding to color intensity signals across four color channels, each generally corresponding to the signal produced by addition of one of the four nucleotides A, T, C or G during a sequencing process. As discussed elsewhere in this specification, the channels may experience color crosstalk.

A model may be built using data obtained using multiple light sensing channels. Each channel may be specific for a specific frequency bandwidth. In some cases, the model may be built using four channels, wherein the bandwidth of each channel may be selected for signals produced by addition of one of the four nucleotides A, T, C or G. In other cases, more or less than four channels may be used to collect data used to produce the model.

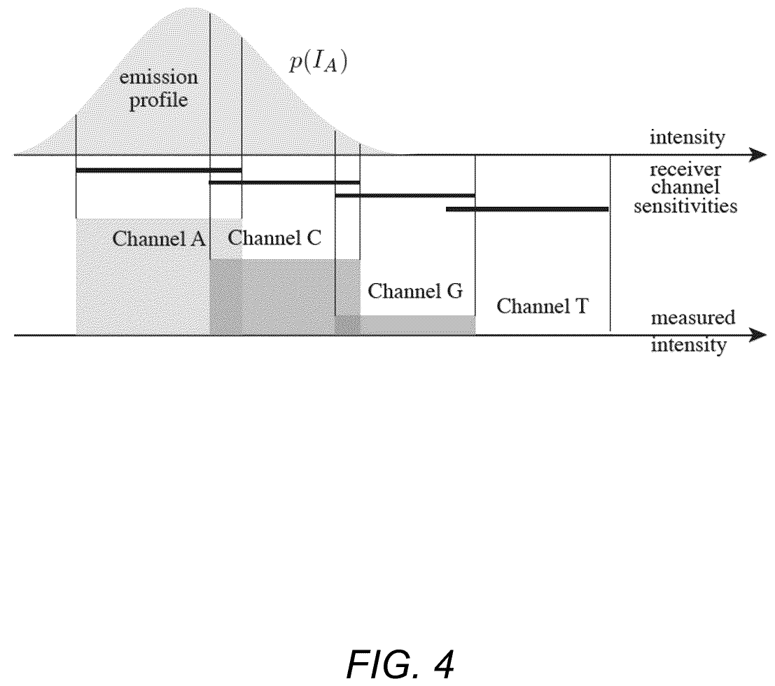

In certain embodiments of the invention, each channel detects a bandwidth region of a fluorescence signal produced by addition of one of the four nucleotides. Nevertheless, the bandwidth of the signal produced by addition of one of the four nucleotides may be spread across a spectral band that overlaps with other channels. This effect is illustrated in FIG. 4. FIG. 4 shows a hypothetical emission spectrum, which is detected at varying intensities by Channel A, Channel C and Channel G, and not detected by Channel T.

As will be discussed in the examples below, a color crosstalk model may be empirically developed and used as input into the model of the invention for producing a probability that a code is present. Relative coefficient strength may be experimentally determined across color channels for signal produced by addition of each nucleotide (A, T, C, G) from empirically produced test data.

Other factors that may be included in a statistical model according to the invention for calculating a probability that a code is present include signal phasing, signal droop, color cross-talk values, fluctuations in in color cross-talk values, noise, amplitude noise, gaussian amplitude models, and base calling algorithms.

The model of the invention may also take into account various sources of noise and error, such as variability in the concentration of the active molecules in the assay, variability in color channel response due primarily to limited ability to estimate the color channel responses individually for each cluster, and background and random error noise sources. A concentration noise model may be used to model the variable density of active molecules for a given cluster. A transduction noise model may be included to model variability in the color crosstalk matrix.

Accurately modeling the biochemical opto-mechanical processes in DNA sequencing is a complex process. Furthermore, to derive the inputs for a soft decision probabilistic signal estimator requires estimating the parameters driving the model, as well as having strong confidence that the model is accurate. Under these two assumptions, metrics can be computed that work directly with the received signals. In the commercially available base call algorithms, channel distortion effects are compensated for before the decision process; however, in soft decision decoding of the invention it is not necessary to compensate for distortions before decoding. Embodiments which do not compensate for distortions before decoding will have the advantage of avoiding information loss compensations, such as inversions.

The probability that a particular code is present may be indicative of the probability that a particular target associated with the probe is present. Data indicating the probability that a particular target is present may be used, for example, to calculate probabilities relevant to diagnosis or screening of various medical conditions, or selection of drugs for treatment of various medical conditions.

The disclosure provides encoded probes that can be decoding using soft decision decoding methods or models. The codes may be generated using the trellis method and the codes may be referred to as “trellis codes”. The probes of the invention may be padlock probes that include a soft decodable code, such as a trellis code. The probes of the invention may be a dual probe that includes a soft decodable code, such as a trellis code. In some cases, mixtures of encoded probes include sets of 10, 100, 1000, 100000, 1 million or more codes in which each probe of the set includes a soft decodable code.

The disclosure provides assays that make use of encoded probes that may be decoded using soft decision decoding (“soft decoding”). In various embodiments, the assays make use of mixtures of probes, each with a soft decodable code. A mixture may include 100s, 1000s, or 10000s of encoded probes.

In some instances of the methods of the invention, determining the presence of or the sequence of the code is performed without making a specific base call for each nucleotide in the code.

In some embodiments, a hybridization-based detection method may be used to decode the code. In one embodiment, the amplified codes are decoded using oligonucleotide probes in a hybridization-based reaction. The amplified codes may be decoded using sequencing by hybridization. In one example, the hybridization-based detection method uses fluorescently labeled oligonucleotide probes. The code data may then be used as a digital count of the target-specific detection events.

### Methylation Assays

The invention makes use of recognition elements and encoded oligonucleotide probe sequences (“encoded probes”) for detecting a panel of target nucleic acids in a sample (e.g., a blood sample or fraction thereof), wherein the encoded probes are used as surrogates for bioanalysis of the targeted nucleic acids.

A methylation assay using encoded probes (i.e., an encoded assay) may include: (i) a recognition event, in which a target nucleic acid is uniquely recognized and bound by a recognition element associated with a code (i.e., an encoded probe); (ii) a transformation event, in which a molecular transformation of the recognition element produces a modified recognition element comprising the code; and (iii) a detection event, that uses the code as a surrogate for detection of the target nucleic acid, e.g., by recognizing or determining the presence or the sequence of the code (and optionally other elements).

In one embodiment, an encoded methylation assay may be performed in a plate-based format, such as a multi-well plate. The multi-well plate may include, for example, an array of nanowells.

In one embodiment, an encoded methylation assay may be performed on a microfluidics device.

An encoded methylation assay may be a solution-based assay.

An encoded methylation assay may be a surface-bound assay.

An encoded methylation assay may be a hybrid assay that includes a surface-bound component and a solution-based component.

An encoded probe may include other functional sequences such as sequencing primers, one or more amplification primer sequences, unique identifier sequences (UMIs) and sample indexes. The sequencing primers may be adjacent to the code sequence. The amplification primer sequences may be universal primer sequences that are common to all probes in a set of encoded probes.

An encoded probe may be a coded padlock probe that includes a recognition element associated with a code. The code may be a soft decodable code, such as a trellis code.

An encoded probe may be a split or dual oligonucleotide probe, wherein a ligation event is used to generate an amplifiable or readable code. The code may be a soft decodable code, such as a trellis code.

The detection event may include an amplification step in which the code sequence (among other elements) is amplified. Amplification may be by any method of amplification, including for example, on-surface PCR, isothermal amplification, rolling circle amplification, and/or ultrarapid amplification. Surface based amplification may be performed using PCR with surface-anchored primers (e.g., Illumina bridge amplification technology) or recombinase polymerase amplification (RPA) (e.g., ExAmp technology).

In one embodiment, the amplification step comprises a rolling circle amplification reaction (RCA) to generate a nanoball output product.

In one embodiment, an encoded probe may include a sequence which may not be extended in an amplification reaction. The non-extendable sequence may, for example, be located between a pair of amplification primer sequences. In some embodiments, incorporating a non-extendable sequence into the encoded probe may prevent generic rolling circle amplification (RCA) of the padlock probe, thereby allowing for linear double-stranded PCR products.

In one embodiment, an encoded probe may include a restriction enzyme site that may be cleaved to yield a linear DNA molecule.

In the detection event, the amplified code may in certain embodiments be sequenced to determine the sequence of the code associated with the target nucleic acid. Any sequencing technology may be used to sequence. Examples of sequencing technologies that may be used include sequencing by synthesis (e.g., pyrosequencing; sequencing by reversible terminator chemistry (Illumina)), avidity sequencing (Element Biosciences), sequencing by hybridization, sequencing by ligation, and nanopore sequencing.

In some embodiments, a sequencing library may be generated from a set of modified recognition elements comprising the codes. The library may be sequenced to decode the code associated with a target of interest. The code data may then be used as a digital count of the target-specific detection events.

In one embodiment, a sequencing library comprising the code (among other elements) may be generated from a circularized padlock probe.

In one embodiment, a sequence library comprising the code (among other elements) may be generated from a nanoball product.

In one embodiment, a nanoball or a portion of the nanoball that includes the code (and optionally other elements) may be directly sequenced to decode the code associated with the target of interest. The code data may then be used as a digital count of the target-specific detection events.

In some embodiments, a hybridization-based detection method may be used to decode the code. In one embodiment, the amplified codes are determined using oligonucleotide probes in a hybridization-based reaction such as, for example, sequencing by hybridization. In one example, the hybridization-based detection method uses fluorescently labeled oligonucleotide probes. The code data may then be used as a digital count of the target-specific detection events.

In one embodiment, a methylated target sequence may be detected using a methylation-specific inhibition of ligation reaction. For example, methylation specific inhibition of ligation may be based on the use of a protein that binds specifically to methylated nucleic acid groups thereby interfering with the activity of the ligase and preventing the circularization of a coded padlock probe. In another example, methylation-specific aptamers may be used for methylation specific inhibition of ligation reaction.

In one embodiment, a methylated target sequence may be detected using a padlock probe ligation reaction in combination with a methylation-specific restriction enzyme digestion reaction.

In one embodiment, dual oligonucleotide probes in combination with a methylation sensitive endonuclease digestion reaction may be used to differentiate a methylated target sequence and an unmethylated target sequence.

In one embodiment, a proximity-based ligation process using a linear detection probe in combination with a modified methyl binding protein (e.g., a methylation specific antibody or fragment thereof) and a location specific oligonucleotide probe may be used for detection of a methylation site in a target sequence of interest.

### Coded Padlock Probes

The disclosure provides assays that make use of novel padlock probes comprising codes that may be used as a surrogate for detection of a target, e.g., by recognizing or determining presence of or the sequence of the code (and optionally other elements). The code in a padlock probe may be a soft decodable code (e.g., a trellis code). A coded padlock probe may include target-specific regions that may be used for target recognition and enrichment. A coded padlock probe may include a 5′ terminal phosphate that may be used to facilitate ligation (i.e., circularization) after target recognition. A coded padlock probe may include a 3′ nucleotide that is the complement to a nucleotide at a target site of interest (e.g., a 3′ SNP-specific nucleotide). A coded padlock probe may include an RCA priming site that includes a primer sequence suitable for priming an RCA reaction.

For example, the coded padlock probe may include regions at the 3′ and 5′ ends that are complementary to regions of a target. The probe regions may hybridize to the target, and the probe may be circularized, e.g., by a ligation or gap-fill ligation reaction. As described elsewhere in this disclosure, the target may be a nucleic acid analyte (e.g., mRNA, cfDNA etc.) or a proxy for the analyte of interest (e.g., an antibody conjugated with oligonucleotide).

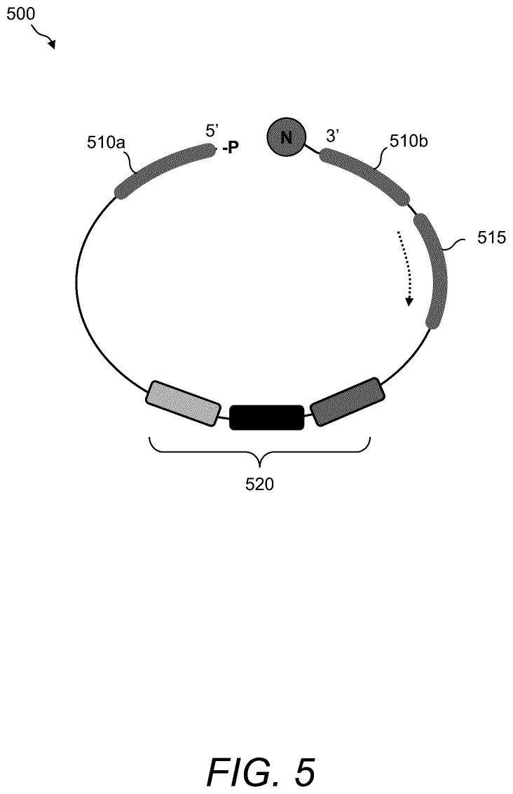

FIG. 5 is a schematic diagram of an example of a coded padlock probe 500. Coded padlock probe 500 may include a 5′ target specific region 510a and a 3′ target specific region 510b that are complementary to regions of a target (not shown). The target may be a nucleic acid analyte (e.g., mRNA, cfDNA, etc.) or a proxy for the analyte of interest (e.g., an antibody conjugated with an oligonucleotide). Target specific region 510a may include a 5′ terminal phosphate (P) that may be used to facilitate ligation (i.e., circularization) after target recognition. Target specific region 510b may include a terminal 3′ nucleotide “N” that is the complement of a nucleotide at a target site of interest. In this example, the 3′ target site specific nucleotide “N” may be a SNP specific nucleotide.

Target specific regions 510a and 510b may hybridize to the target, and the probe may be circularized. For example, when the complementary nucleotide is present in the target, the 3′ SNP specific nucleotide hybridizes to the target, enabling circularization, e.g., by ligation or gap-fill ligation. Other types of features or mutations may be detected by varying the terminal nucleotide (N) or nucleotides of target specific region 510a and/or target specific regions 510b to hybridize when the target feature is present and not hybridize when the target feature is not present.

Coded padlock probe 500 may include an RCA priming site 515 that includes a primer sequence suitable for priming an RCA reaction. In this example, RCA priming site 515 is downstream from target specific region 515b. However, other locations are possible, as long as the positioning the primer site doesn't interfere with the other functions of the probe, e.g., the probe hybridization function and the encoding function.

A coded padlock probe may optionally include other functional sequences. For example, the probe may include index sequences which are unique oligo identifiers present in the probe sequence or inserted as part of the assay. Index sequences, such as sample barcodes, allow differentiation among different samples, experiments, etc. during the detection event (i.e., reading (decoding) the code).

The coded padlock probe may include unique molecular identifiers. UMIs may be inserted anywhere within the probe to address downstream readout and data analysis purposes. For example, UMIs may be introduced to distinguish unique recognition events with single-molecule resolution during the readout. UMI's may facilitate error correction and/or individual molecule counting.

A coded padlock probe may include other primers in addition to the priming region required for RCA amplification. Other priming regions may, for example, be present to facilitate the readout of an index, a UMI or other oligonucleotide sequences present in the probe. Priming regions may allow parallel or serial reading schemes. They may also be used to increase the amount of multiplexing or allow sequential readout. For instance, if a plurality of probes or amplified objects are present, only those containing a specific primer will be amplified or read. Primers may also be used to facilitate the capture and immobilization of a probe or amplified object onto a surface (e.g., via DNA-DNA hybridization).

A coded padlock probe may include one or more sequences recognizable by enzymes, such as endonucleases. Various sequences may be selected and used to facilitate additional transformations, such as digestion, nick or gap formation, phosphorylation etc. In one embodiment, the probe includes one or more restriction sites.

A coded padlock probe may include one or more non-natural NTP components. Examples include phosphorothioate groups, locked DNA (LNA), peptide DNA (PNA) and others, which may be included to improve certain features of the probe, such as melting temperature for target recognition, or primer recognition, or resistance to degradation. Additionally, abasic NTPs (“wobble bases”) may be included in the probe sequence to add degeneracy to targeting or priming regions and extend the ability to recognize a broader number of complementary sequences.

A coded padlock probe may include one or more chemical moieties. Such chemical moieties may be included in the probe structure or added at any stage of the workflow to enable additional transformations or properties. Examples include cleavable groups to open or linearize the probe, reactive groups to add additional components such as dyes, and groups to facilitate immobilization on surfaces.

A coded padlock probe may include CRISPR recognition sequences, oligo sequences designed to be recognized by CRISPR enzymes and replaced with other arbitrary sequences. The probe may optionally include one or more oligo sequences designed to be recognized by transposases and replaced with other arbitrary sequences.

A coded padlock probe may optionally include one or more adapter primers for compatibility with sequencing by synthesis (SBS) and other non-SBS platforms. The adapter primers may be included in the probe sequence or added at any stage as part of the workflow. Such adapter primers may be used directly to immobilize, cluster, extend, and amplify as precursor activities to a decoding run by SBS.

In one embodiment, a padlock probe assay workflow may include:
(i) hybridizing the probe to a target;
(ii) optionally, extending the hybridized probe to fill any single-stranded gap remaining between the two probe arms;
(iii) circularizing the probe when the target analyte is present;
(iv) cleaning-up (e.g., by exonuclease or other mean) non-circularized probes remaining after ligation;
(v) amplifying the circularized probe by RCA or other methods;
(vi) capturing of the amplified product on a surface;
(vii) degrading the amplified product to generate a sequencing compatible library;
   preparing the library for sequencing, using sequencing sample preparation workflows suitable for a desired sequencing platform; and reading out or decoding the code.

### Index Sequences

Index sequences, such as sample barcodes, allow differentiation among different samples, experiments, etc. during the detection event (i.e., reading (decoding) the code). Indexes may be added to a padlock probe using a variety of strategies.

Indexes may be added during the synthesis of a padlock probe. In this case, for every probe manufactured, the number of probes is N×P, where N is the number of indices and P is the plexity of the probe pool.

Indexes may be added after probe synthesis as part of manufacturing or at a site of use as a step prior to performing an encoded assay. In this case, only one synthesis is required for each probe and additional functional elements. Additional functional elements may be added to a probe to enable insertion of an index. Examples of functional elements that may be added include (i) non-natural nucleotides (e.g., biotin, amine, etc.) and (ii) polynucleotides that enable biochemical transformation of the probe to contain an index sequence such as adapters for ligations or extension ligations, restriction endonuclease recognition sites, and transposome binding sites.

Indexes may be added during an encoded assay. For example, a ligation reaction to insert an index can occur at the same time as ligation of the padlock probe at the target site of interest to generate a circularized padlock probe (i.e., the transformation event). In some cases, the ligation reaction may be a gap-fill extension/ligation reaction.

Indexes may be added after ligation of the padlock probe and RCA by including modified nucleotides during the RCA reaction. The modified nucleotides may then be coupled to an index sequence. In cases where there is a covalent or non-covalent interaction, either moiety can be linked to the index sequence or incorporated during RCA.

Examples of coupling strategies include: (i) ligand protein pairs such as biotin-streptavidin, antigen-antibody, CLIP tag and SNAP tag pair (i.e., O6-benzylguanine derivatives coupling to O6-alkylguanine-DNA-alkyltransferase, wherein either the protein or the substrate may be bound to the probe), carbohydrate-protein pairs (e.g., lectins), and digoxigenin-DIG-binding protein; (ii) peptide-protein pairs (e.g., SpyTag—SpyCatcher); and (iii) hybridizing indexes to a common sequence on the RCA product.

Indexes may be added to RCA products by restriction endonuclease cleavage followed by index ligation.

Indexes may be added to RCA products using a transposase enzyme that fragments and indexes the RCA products.

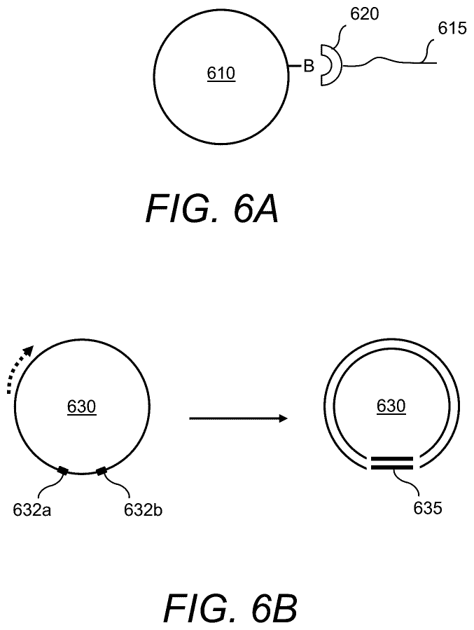

FIG. 6A is a schematic diagram illustrating an example of adding an index sequence to a probe using a ligand protein coupling strategy. In this example, the ligand protein pair is biotin—streptavidin. Biotinylated nucleotides “B” may be incorporated into a probe 610 and an index sequence 615 may be attached to a streptavidin protein 620. Index sequence 615 may then be coupled to probe 610 via formation of a streptavidin—biotin linkage.

FIG. 6B is a schematic diagram illustrating an example of adding an index sequence to a probe by restriction endonuclease cleavage followed by index ligation. A probe 630 may include a pair of restriction sites 632a and 632b. A polymerase extension reaction (indicated by dashed arrow) may be performed to convert probe 630 to a double-stranded molecule prior to cleavage. An index sequence 635 may be added to probe 630 by restriction endonuclease cleavage followed by index ligation.

### Surface attachment

The encoded assays of the invention may be performed on a surface. For example, a target may be immobilized on a surface for conducting assays of the invention. The probes of the invention may be immobilized on a surface for conducting assays of the invention. DNA nanoballs of the invention may be immobilized on a surface for conducting assays of the invention. Various intermediate assemblies of molecules of the assays of the invention may be immobilized on a surface for conducting assays of the invention.

Various steps of the invention may be performed on a surface, such as target capture, recognition events, transformation events, amplification, and/or detection events, i.e., determination of the absence or presence of the code (e.g., by sequencing or hybridization-based detection).

Thus, for example, the disclosure provides a surface having a probe as described herein immobilized on the surface. The disclosure provides a surface having a nanoball as described herein immobilized on the surface. The disclosure provides a surface having a target immobilized on the surface. The disclosure provides a surface having a target immobilized on the surface with a probe as described herein hybridized to the target. The disclosure provides a surface having a probe immobilized on the surface with a target as described herein hybridized to the probe. The disclosure provides a surface having a target nucleic acid immobilized on the surface, and a protein or peptide bound to the target nucleic acid. The disclosure provides a surface having a target nucleic acid immobilized on the surface, and an antibody, aptamer, binder, or antibody fragment bound to the target nucleic acid. The disclosure provides a surface having a ligand that has affinity for any of the foregoing immobilized on the surface. For example, the ligand may have affinity for a probe as described herein, a nanoball as described herein, or a target as described herein. The ligand may, for example, be a protein, peptide, antibody, aptamer, binder, or antibody fragment.

A variety of surfaces may be used for the surface attachments described herein. In various embodiments, the surface includes an oxide, a nitride, a metal, an organic or an inorganic polymer (e.g., hydrogel, resin, plastic or other).

The surface may take a variety of forms, e.g., it may be flat or curved. It may be beads or particles. In some cases, the surface is the surface of a flow cell. Beads or other particles may in some embodiments range in size from less than 100 nm up to several centimeters.

Various surface modifications may be used to permit attachment of various components of the assays of the invention to a surface. For example, various anchoring ligands may be used (e.g., streptavidin, biotin, aptamers, antibodies, etc.). Chemical handles, such as click chemistry handles, may be used. Examples include azides, alkynes, unsaturated bonds, amines, carboxylic acids, NHS, DBCO, BCN, tetrazine, epoxy and the like. Single-or double-stranded oligonucleotides may be used. Size ranges of the oligonucleotides may, in some cases, be from about 10 to about 200 nucleotides. Proteins or peptides may be used for surface attachment. Charge-based molecules or polymers may be used, e.g., polyethylenimine.

Various techniques may be used to prepare a surface for binding to a target or to a component of an assay of the invention. In one example, a flow cell with primers may be used. A splint DNA segment that comprises a segment complementary to the primer and a segment that is complementary to the target or the component of the assay may be hybridized to the primer. A variety of splints may be used on a surface, with various subsets of the splints having different segments complementary to different components of the invention or different targets. Specific splints may be arranged on different regions of a surface. For example, splints may be arranged in a manner that permits the identification of distinct regions of a surface targeted to specific analytes or components of the assays.

In various embodiments, amplification of a nucleic acid may occur on the surface. The nucleic acid may be a target or any nucleic acid component of an assay of the invention. For example, a target analyte may be amplified on a surface, or a probe of the invention may be amplified on a surface, and/or a fragment of any of the foregoing may be amplified on a surface. The amplification may be performed on a bead or particle, or on a flat surface, such as on the surface of a flow cell.

It should also be noted that DNA may be amplified in solution, e.g., in an aqueous suspension or emulsion, such as in microdroplets. Solution-based amplification may be performed, for example, in an open environment, such as the well of the microtiter plate, in a nanowell, or in an enclosed space, droplet in an emulsion, or on a flow cell or other microfluidic device.

Amplification may be by any method of amplification, including for example, PCR, isothermal amplification and/or ultrarapid amplification.

Attachment for immobilization of components of the assays or of targets may be covalent or non-covalent (e.g., Coulombic in nature), temporary or permanent, and/or rendered labile when subject to a particular stimulus.

Examples of mechanisms of lability include:
Enzymatic—protease, restriction endonuclease, CRISPR-Cas9
Chemical—reduction, hydrolysis, nucleophilic attack, displacement, reducing of a disulfide bond
Temperature—melting of duplexed hybridized DNA, thermodynamically unfavorable conditions (Positive deltaG)
pH—hydrazone, carbonate, etc.
Light—O-nitrobenzyl or derivatives where absorption of light of a particular wavelength(s) can cause bond rearrangements or cleavage. Light sensitive groups include nitro-benzene derivatives
Ligand mediated—competitive competition for binding site (see examples below)
   Peptide—tagged oligos with protein interactions—e.g., Spy-catcher. The moiety may be the ligand or the protein.
   Peptide—tagged oligo with heavy metal interactions—e.g., Hexa-histidine—to Cu. The moiety may be the ligand or the protein.
   CLIP tag and SNAP tag pair—i.e., O6-benzylguanine derivatives coupling to O6-alkylguanine-DNA-alkyltransferase. Either the protein or the substrate may be bound to the oligo.
   Carbohydrate—protein pairs, e.g., lectins
   The moiety may be a ligand (e.g., biotin, digoxigenin) coupled to a fluorescently-tagged protein (e.g., avidin, streptavidin, DIG-binding protein)
Cleavage can be performed by cleaving a moiety dangling on a nucleotide, or a nucleotide or a nucleobase within the oligo sequence or the di-nucleotide linkage, e.g., uracil and USER cocktail (uracil-N-deglycosylase (UNG)) followed by Endonuclease VIII or FPG (Formamidopyrimidine DNA Glycosylase with Bifunctional DNA glycosylase with DNA N-glycosylase and AP lyase activities)
Cleavage can be performed by an enzyme

### Surface-Based Workflows

A variety of surface-based workflows are possible within the scope of the assays disclosed. In some embodiments, a surface-based workflow may use a padlock probe that includes a recognition element associated with a code. The code may be a soft decodable code, such as a trellis code. In some embodiments, a surface-based workflow may use a dual probe that includes a recognition element associated with a code (e.g., a trellis code).

In some embodiments, a surface-based workflow may include immobilizing a target on a surface and hybridizing a probe to the target. In one embodiment, a surface-based workflow may include:
(i) immobilizing the target on a surface;
(ii) hybridizing a probe to the immobilized target;
(iii) circularizing the probe to produce a circular modified probe; and
(iv) releasing the circular modified probe from the target.

In some embodiments, the target may be a nucleic acid, e.g., DNA. In this case, immobilization of the nucleic acid target (e.g., DNA) may be at an end of the target or via a side chain or internal segment of the target.

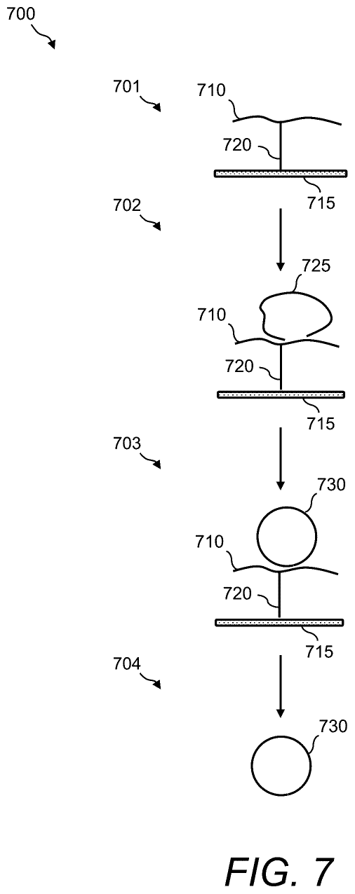

FIG. 7 is a schematic diagram of an example of a surface-based workflow 700, wherein the target is immobilized on the surface and used to template the ligation of a probe to produce a circular modified probe. Workflow 700 may include, but is not limited to, the following steps.

In a step 701, a target is immobilized on a surface. For example, a target 710 is immobilized on a surface 715 by an anchor element 720. In one example, target 710 is DNA and anchor element 720 is an oligonucleotide.

In a step 702, a linear probe is hybridized to the immobilized target. For example, a solution that includes a probe 725 is added and a hybridization reaction is performed to bind probe 725 to target 710. In one example, probe 725 is a coded padlock probe.

In a step 703, the probe is circularized. For example, a ligation reaction is performed to circularize probe 725 to produce a circular modified probe 730. In some cases, a gap-fill extension/ligation reaction is used to circularize probe 725 to produce the circular modified probe.

In a step 704, the circular modified probe is released from the immobilized target for downstream processing. For example, circular modified probe 730 may be dehybridized from target 710 and amplified in an RCA reaction to produce a nanoball product.

In some cases, the RCA reaction may be performed in a solution that remains in contact with the surface on which the target is immobilized (e.g., in the same container, well, reservoir, liquid volume or droplet). In some cases, the solution comprising the released modified probe may be transferred to a separate container prior to performing the RCA reaction. In some cases, the solution comprising the released modified probe may be transferred to a different surface prior to performing the RCA reaction.

In some embodiments, the immobilized target (e.g., DNA) may be used to prime the RCA reaction. In one embodiment, a surface-based workflow may include:
(i) immobilizing the target on a surface;
(ii) hybridizing a probe to the target;
(iii) circularizing the probe to produce a circular modified probe; and
(iv) using the target to prime an RCA reaction to generate a nanoball product.

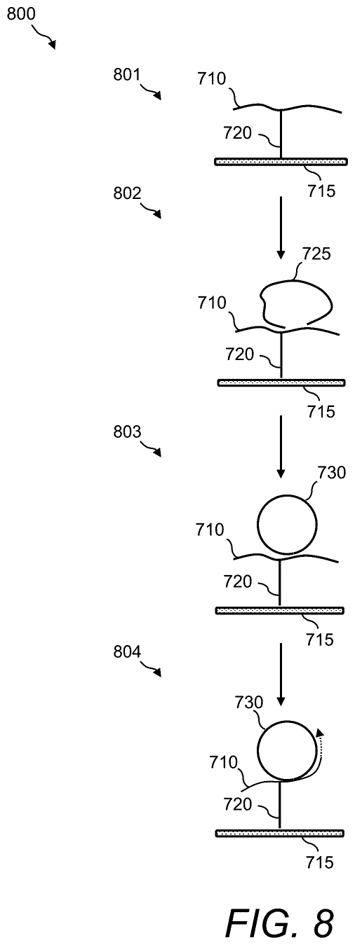

FIG. 8 is a schematic diagram of an example of a surface-based workflow 800, wherein the target immobilized on the surface is used as a primer to initiate the amplification of the probe to generate a nanoball product. Workflow 800 may include, but is not limited to, the following steps.

In a step 801, a target analyte is immobilized on a surface. For example, a target 710 is immobilized on a surface 715 by an anchor element 720. In one example, target 710 is DNA and anchor element 720 is an oligonucleotide.

In a step 802, a linear probe is hybridized to the immobilized target. For example, a solution that includes a probe 725 (e.g., a coded padlock probe) is added and a hybridization reaction is performed to bind probe 725 to target 710.

In a step 803, the probe is circularized. For example, a ligation reaction is performed to circularize probe 725 to produce a circular modified probe 730. In some cases, a gap-fill extension/ligation reaction is used to circularize probe 725 to produce the circular modified probe.

In a step 804, the immobilized target 710 is used to as a primer to initiate an RCA reaction to generate a nanoball product.

In some embodiments, a surface-based workflow may include immobilizing a probe (or a part thereof) on a surface and using the immobilized probe to capture a target. In one embodiment, a surface-based workflow may include:
(i) immobilizing the probe (or a part thereof) on a surface;
(ii) hybridizing a target to the probe;
(iii) circularizing the probe to produce a circular modified probe; and
(iv) using the target to prime an RCA reaction to generate a nanoball product.

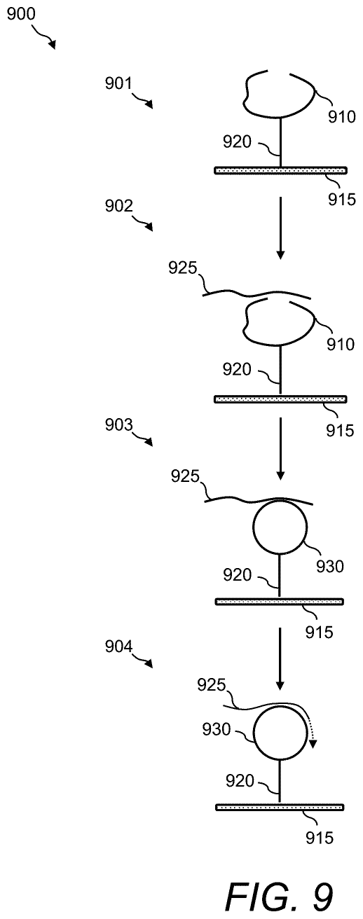

FIG. 9 is a schematic diagram of an example of a surface-based workflow 900, wherein the probe is immobilized on the surface and the immobilized probe is used capture a target. Workflow 900 may include, but is not limited to, the following steps.

In a step 901, a linear probe is immobilized on a surface. For example, a probe 910 is immobilized on a surface 915 by an anchor element 920. In one example, probe 910 is a padlock probe and anchor element 920 is an oligonucleotide.

In a step 902, a target is hybridized to the immobilized probe. For example, a solution that may include a target 925 is added and a hybridization reaction is performed to bind target 925 to probe 910.

In a step 903, the probe is circularized. For example, a ligation reaction is performed to circularize probe 910 to produce a circular modified probe 930. In some cases, a gap-fill extension/ligation reaction is used to circularize probe 910 to produce the circular modified probe.

In a step 904, the circular modified probe is amplified in an RCA reaction to generate a nanoball product. Circular modified probe 930 may be amplified without being released from the surface. For example, circular modified probe 930 may be amplified in an RCA reaction using target 925 as a primer to initiate the amplification reaction.

In some embodiments, the circular modified probe may be released from the surface prior to amplification. In some cases, the RCA reaction may be performed in a solution that remains in contact with the surface on which the probe was anchored (e.g., in the same container, well, reservoir, liquid volume or droplet). In some cases, the solution comprising the released modified probe may be transferred to a separate container prior to performing the RCA reaction.

In some embodiments, the solution comprising the released modified probe may be transferred to a different surface prior to performing the RCA reaction. In one embodiment, oligonucleotides bound to the new surface may be used as capture moieties to immobilize the circular modified probe on the surface and to initiate the amplification reaction. In one embodiment, the target may be immobilized on the new surface and used to initiate the amplification reaction.

A surface-based workflow may use a dual probe as a recognition element. In one embodiment, a surface-based workflow using a dual probe may include:
(i) hybridizing a target to a first probe;
(ii) hybridizing the target to a second probe; and
(iii) performing a ligation or a gap-fill ligation reaction to link the first probe and the second probe.

In some embodiments, the first probe and the second probe may both be immobilized on the surface.

In some embodiments, the first probe is immobilized on the surface and the second probe is in solution. The surface may, for example, be the surface of a flow cell.

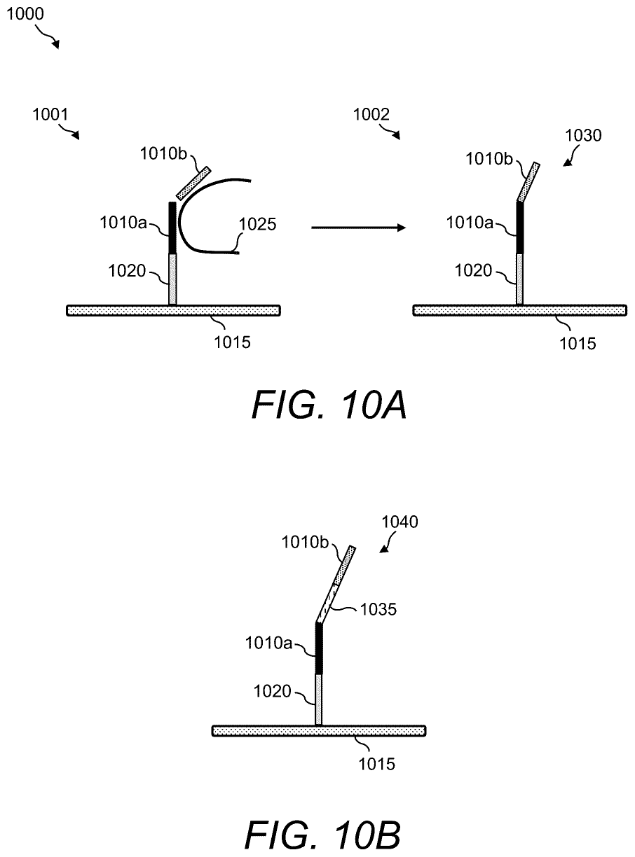

FIG. 10A is a schematic diagram of an example of a dual probe workflow 1000, wherein a first probe is immobilized on the surface and the second probe is in solution. Workflow 1000 may include, but is not limited to, the following steps.

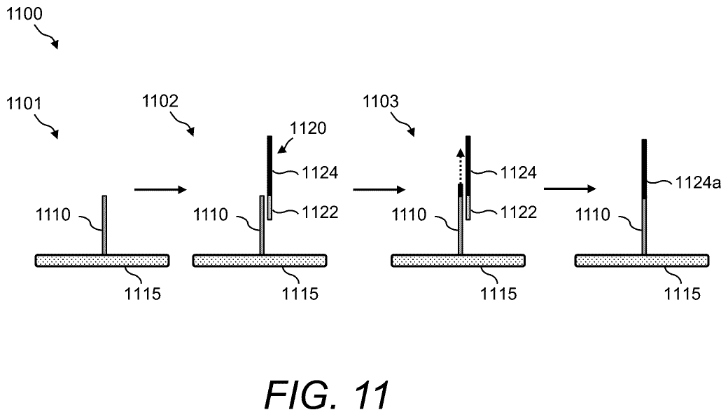

In a step 1001, a target is hybridized to a first probe immobilized on a surface. For example, a first probe 1010a is immobilized on a surface 1015 via an anchor element 1020. In one example, anchor element 1020 is a surface bound primer. The surface bound primer may, for example, be a primer on a sequencing flow cell. A process for anchoring a probe (or a segment thereof) on a surface bound primer is described below with reference to FIG. 11.

First probe 1010a may be used as a capture element for recognizing and binding a target. For example, a solution that may include a DNA target 1025 is added and a hybridization reaction is performed to bind target 1025 to first probe 1010a.

In a step 1002, the target is hybridized to a second probe. For example, a second probe 1010b that includes a sequence for recognizing and binding target 1025 is added and a hybridization reaction is performed to hybridize second probe 1010b to target 1025.

In a step 1003, the dual probe is ligated to link the first probe and the second probe to produce a modified probe immobilized on the surface. For example, a ligation reaction is performed to link first probe 1010a and second probe 1010b to produce a modified probe 1030. In some cases, a gap-fill extension/ligation reaction is used to link first probe 1010a and second probe 1010b to produce the modified probe.

In some cases, second probe 1010b may further include a surface oligonucleotide adapter for binding to another surface bound primer. FIG. 10B is a schematic diagram illustrating an example of a surface bound probe that includes a second surface adapter. In this example, probe 1010b may further include a surface adapter 1035 that is introduced during ligation of probe 1010a and 1010b to produce a modified probe 1040.

The disclosure provides a process for preparing a surface for binding to a target or to a component of an assay of the invention. Surface modifications may serve a dual purpose. For example, a surface modification may (i) capture the target of interest and (ii) initiate the amplification of a probe or a portion thereof on the surface. In another example, a surface modification may (i) capture a component of the assay (e.g., a circular modified probe), and (ii) initiate an RCA reaction to generate a nanoball product.

A surface bound primer may be enzymatically modified to include a capture sequence. A capture sequence may be a target-specific probe or a sequence that is specific for a component of an assay. A surface bound primer may be enzymatically modified to include a probe or a portion thereof (e.g., a probe arm or a primer binding site). For example, a splint oligonucleotide that includes a segment that is complementary to a surface bound primer and a segment that is complementary to a probe (or a portion thereof) may be hybridized to the primer and used to template the synthesis of a surface bound probe. In one example, the surface bound probe is one arm of a dual probe.

FIG. 11 is a schematic diagram illustrating an example of a process 1100 for synthesizing a surface bound probe using a splint oligonucleotide. Process 1100 may include, but is not limited to, the following steps.

In a step 1101, a surface is provided with a surface bound primer. For example, a primer 1110 is bound to a surface 1115. Surface 1115 may, for example, be the surface of a flow cell.

In a step 1102, a splint oligonucleotide is hybridized to the surface bound primer. For example, a splint 1120 that includes a segment 1122 that is complementary to primer 1110 and a capture segment 1124 is hybridized to primer 1110. In one example, capture segment 1124 is one arm of a dual capture probe.

In a step 1103, a primer extension reaction is performed to synthesize the surface bound probe. For example, in the primer extension reaction, splint 1120 is used to template the synthesis of a capture segment 1124 extending from primer 1110 to produce a surface bound probe arm 1124a.

Amplification Strategies

Amplification may be by any method of amplification, including for example, on-surface PCR, isothermal amplification, rolling circle amplification, and/or ultrarapid amplification.

Surface based amplification may be performed using PCR with surface-anchored primers (e.g., Illumina bridge amplification technology) or recombinase polymerase amplification (RPA) (e.g., ExAmp technology).

Clonally amplified material may be a nanoball or a DNA cluster (e.g., Illumina surface-based amplification).

An amplification strategy may include adding a second surface adapter to a probe. The second surface adapter may be complementary to a second primer on a flow cell surface (e.g., a bridge amplification primer). The second surface adapter may, for example, be added to a probe during the ligation or gap-fill ligation event or added separately by PCR or through its own ligation to a probe. For example, an amplification strategy may include using the splint ligation approach described with reference to FIG. 11 to add a second surface adapter to a surface bound probe to facilitate bridge amplification. Bridge amplification may be used to create clusters for sequencing.

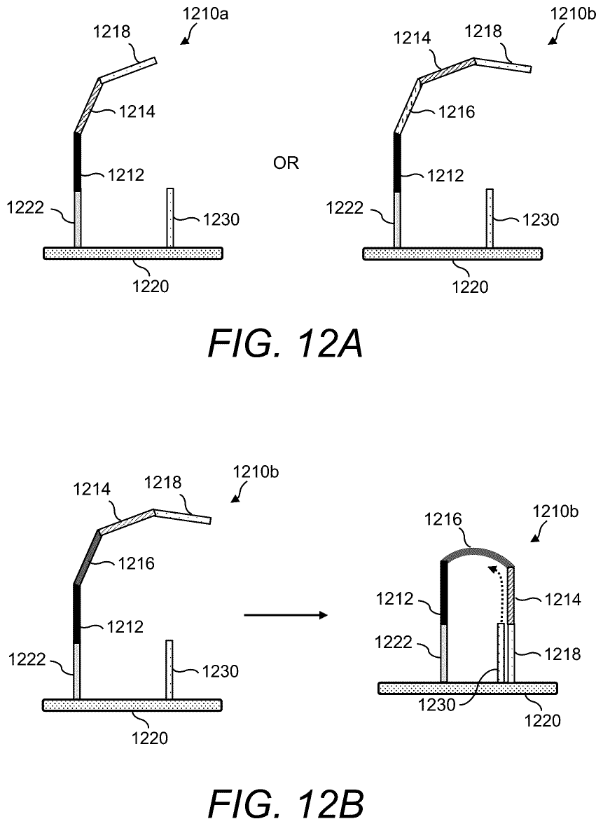

FIG. 12A and FIG. 12B are schematic diagrams illustrating an example of surface bound probe structures comprising a second surface adapter and showing a process of performing bridge amplification, respectively. In this example, the probe structure is a modified dual probe as described above with reference to FIG. 10, wherein the probe further includes a second surface adapter for bridge amplification. For example, a probe structure 1210 (i.e., probe structures 1210a and 1210b) may be anchored to a surface 1220 by a primer 1222. Primer 1222 may be a primer used in a bridge amplification reaction. Probe structure 1210a may include a first probe element 1212, a second probe element 1214, and a surface adapter 1218. Surface adapter 1218 may be complementary to a second primer 1230 on surface 1220. Second primer 1230 may be a primer used in a bridge amplification reaction. Probe structure 1210b may include first probe element 1212 and second probe element 1214 that are separated by an adapter 1216, and surface adapter 1218. A bridge amplification reaction (see FIG. 12B) may be performed to create clusters (not shown) for sequencing.

An amplification strategy may include adding a restriction enzyme site in a probe. For example, the probe may include a restriction enzyme site that when hybridized with a complementary oligonucleotide provides a double-stranded site for a restriction endonuclease to cleave the probe, rendering a linear strand. The linear strand may be amplified for downstream processing, e.g., for sequencing. For example, the linear strand may be captured on a flow cell and amplified by bridge amplification (e.g., Illumina bridge amplification technology) or recombinase polymerase amplification (RPA) (e.g., ExAmp technology).

The probe may include surface primers or surface adapter sequences that are complementary to surface bound primers of a flow cell. The adapter sequences may be linked to or adjacent to the restriction site, so that when the site is cut by a restriction enzyme the linear strand is ready for sequencing. As noted, other forms of cleavage are possible, such as CRISPR mediated cleavage or any other double-stranded break inducing protein.

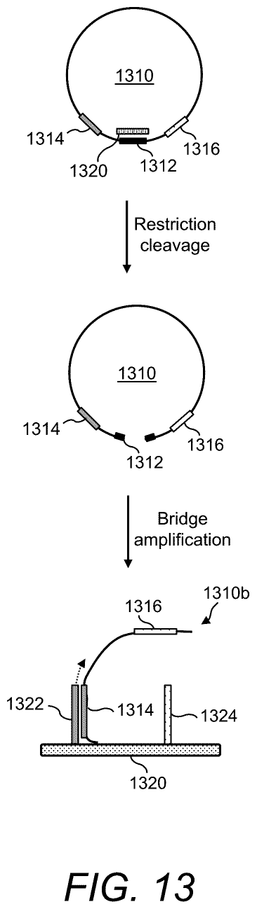

FIG. 13 is a schematic diagram illustrating an example of a probe that includes a restriction enzyme site that may be used to linearize the probe for capture on a flow cell for bridge amplification prior to sequencing. For example, a probe 1310 may include a restriction site 1312. Restriction site 1312 may be linked to a first surface adapter 1314 and a second surface adapter 1316. An oligonucleotide 1320 that is complementary to restriction site 1312 may be hybridized to probe 1310 to provide a double-stranded site for restriction endonuclease cleavage. Cleavage at restriction site 1312 generates a linear probe 1310b. Linear probe 1310b may be loaded on a surface 1320 (e.g., a flow cell surface) that includes a first primer 1322 and a second primer 1324 immobilized thereon. Hybridization of adapter 1314 to primer 1322 may be used to initiate a bridge amplification reaction to generate clusters for sequencing.

Similarly, a nanoball may include surface primers or sequencing adapters linked to or adjacent to a restriction site, so that when the site is cut by a restriction enzyme the linear strands are released ready for sequencing. As noted, other forms of cleavage are possible, such as CRISPR mediated cleavage.

In another embodiment, a nanoball with adapter sequences complementary to surface bound primers may be seeded directly onto the surface without cleaving. Amplification may proceed through bridge amplification (e.g., Illumina bridge amplification technology) or recombinase polymerase amplification (RPA) (e.g., ExAmp technology) initiated directly.

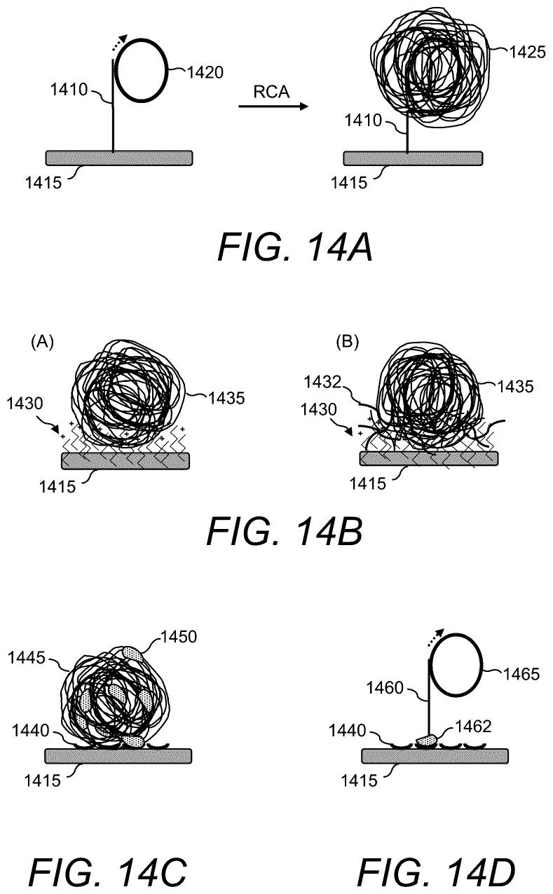

Rolling circle amplification (RCA) may be used to produce nanoballs as part of the assays of the invention. An RCA reaction may be performed as a surface-bound reaction. For example, RCA may be initiated by an oligonucleotide bound to a surface (e.g., beads, flow cells, microwell, or nanowells). Any method may be used to bind the oligonucleotide to the surface. In one example, the oligonucleotide may be covalently bound to the surface. FIG. 14A is a schematic diagram illustrating an example of a process of using a surface-bound oligonucleotide to initiate an RCA reaction (indicated by the arrow). An oligonucleotide 1410 may be covalently attached to a surface 1415. Oligonucleotide 1410 may include an RCA primer sequence that is complementary to an RCA primer site on a probe 1420. Oligonucleotide 1410 may be used to capture probe 1420 by hybridization of the complementary sequences and initiate the RCA reaction. Because oligonucleotide 1410 is covalently bound to the surface, the surface-bound RCA reaction generates a nanoball 1425 that is covalently attached to the surface.

In another example, a cation-coated surface (e.g., beads, flow cells, microwells, or nanowells) may be used to capture nanoballs. In one example, the cation-coated surface may be a polylysine-coated surface. FIG. 14B is a schematic diagram illustrating an example of capturing a nanoball on a cation-coated surface. A surface 1415 may be coated with a polylysine coating 1430. An RCA reaction may be performed in the presence of the polylysine coated surface, resulting in simultaneous immobilization and amplification of a nanoball 1435. RCA primers may be supplied in solution (panel A) or bound to the polylysine-coated surface prior to performing the RCA reaction (panel B).

In another example, a streptavidin-coated surface (e.g., beads, flow cells, microwells, or nanowells) may be used to capture nanoballs. In this approach, biotin-linked deoxynucleotides may be incorporated into the nanoballs during RCA. The nanoballs will then be bound to the surface by a biotin-streptavidin linkage. FIG. 14C is a schematic diagram illustrating an example of capturing a nanoball on a streptavidin-coated surface. A surface 1415 may be coated with a streptavidin coating 1440. An RCA reaction may be performed in the presence of the streptavidin coated surface using biotin-linked deoxynucleotides to produce a nanoball 1445 that includes biotin moieties 1450 resulting in simultaneous immobilization and amplification of nanoball 1445.

In another embodiment, biotin linked RCA primers may be bound to a surface by a streptavidin-biotin linkage and used to initiate an RCA reaction as described above with reference to FIG. 14A. An example of using a biotin-streptavidin linkage to perform a surface-bound RCA reaction is shown in FIG. 14D. A surface 1415 may be coated with a streptavidin coating 1440. An oligonucleotide 1460 that includes a biotin moiety 1462 may be attached to surface 1415 through a biotin-streptavidin linkage. Oligonucleotide 1416 may include an RCA primer sequence that is complementary to an RCA primer site on a probe 1465. Oligonucleotide 1460 may be used to capture probe 1465 by hybridization of the complementary sequences and initiate the RCA reaction (indicated by the arrow) to produce a nanoball. Amplification in the presence of the streptavidin coated surface further anchors nanoball to the surface.

Following the formation of a nanoball, a determination may be made with respect to the identity of the code. Prior to making the determination, various secondary processing steps are possible within the scope of the assays described herein. The probe may include various elements that facilitate secondary processing steps. Examples include restriction endonuclease sites and CRISPR sites.

The nanoball may be converted to double-stranded DNA (dsDNA) prior to fragmentation. The dsDNA nanoball may be fragmented. In one embodiment, the probe includes restriction sites which are replicated in the nanoball, and the nanoball is fragmented using a restriction enzyme having specificity for the restriction sites.

CRISPR may be used to fragment the nanoball at specific sites.

Random fragmentation of nanoballs may be performed, using known fragmentation techniques.

Tagmentation may be performed on the nanoball, and the tagmentation may be used to add sequencing adapters.

### Sequencing Preparation

This disclosure provides a variety of techniques for amplifying and preparing circularized probes for sequencing. In certain embodiments, amplification and preparation for sequencing may be performed sequentially (e.g., PCR+primer ligation). In certain embodiments, amplification and preparation for sequencing may be performed in a single reaction (e.g., adapter addition via PCR). Addition of sequencing adapters may be performed with or without RCA amplification of circularized probes.

In one embodiment, sequencing adapters are added via PCR. In this case, amplification and preparation for sequencing may be a single step. Depending on the probe design, the code, UMI, and index may be read in a single step or in two separate reads with a dehybridization step.

In one embodiment, RCA products (nanoballs) may be fragmented with restriction endonucleases (RE) to yield a multitude of code-containing single stranded nucleic acids. The single-stranded nucleic acids (i.e., the RE reaction products) may then be prepared for sequencing by ligation to adapter sequences.

In one embodiment, sequencing adapters may be added by transposomes that simultaneously fragment double-stranded DNA and add adapters.

As discussed elsewhere in the application, the assays of the invention include a transformation step. Typically, the transformation involves circularization of a probe when a target is present (e.g., by ligation or gap-fill ligation).

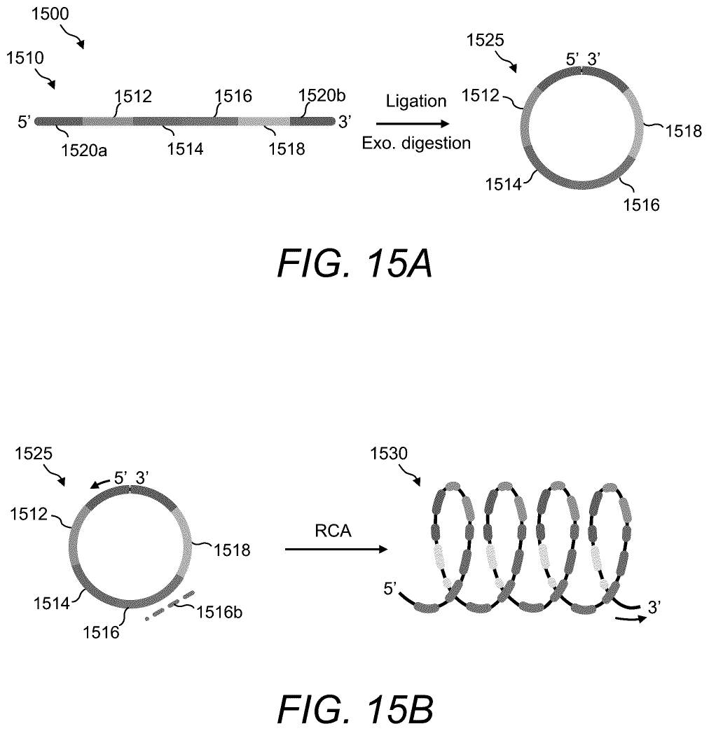

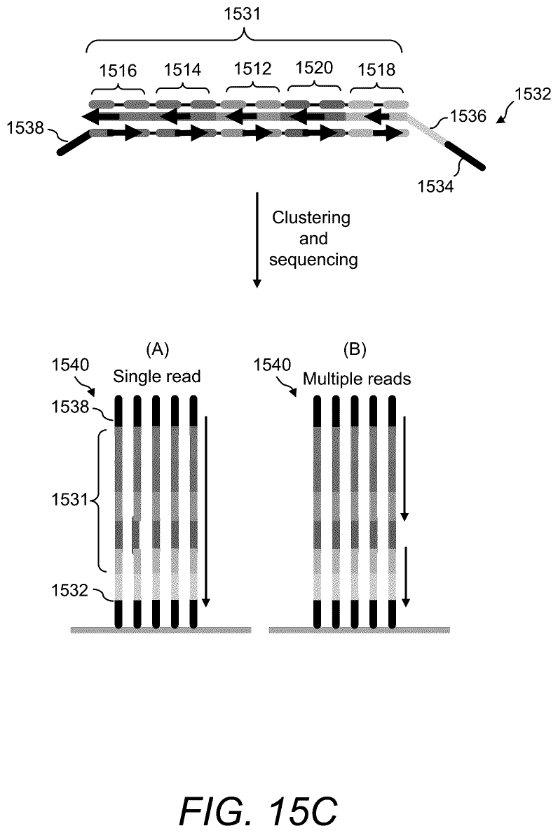

FIG. 15A is a schematic diagram of a transformation process 1500 for circularizing a linear probe to form a circular modified probe. In this example, a probe 1510 includes a UMI sequence 1512, a code 1514, an SBS primer 1516, and an index primer 1518 all situated between a 5′ target recognition element 1520a and a 3′ target recognition element 1520b. In the presence of a target (not shown), probe 1510 is circularized in a ligation reaction to yield a circular modified probe 1525. The ligation reaction may be followed by an exonuclease digestion step to remove unligated probes 1510 and target.

The circular modified probe shown in FIG. 15A may, in some cases, be amplified in a rolling circle amplification to form a nanoball product. FIG. 15B is a schematic diagram showing RCA amplification of the circular modified probe to yield a nanoball product. For example, in an RCA reaction an SBS primer 1516b that is the reverse complement to SBS primer 1516 may be hybridized to circular modified probe 1525 and used to initiate the RCA reaction to generate a nanoball 1530. Nanoball 1530 is a polymeric molecule (concatemer) that includes multiple repeated copies of circular modified probe 1525, wherein each copy includes SBS primer 1516, code 1514, UMI sequence 1512, target recognition elements 1520, and index primer 1518. In this example, the complement (i.e., copy) of modified probe 1525 is indicated by the dashed line.

In some embodiments, the RCA products (nanoballs) may be sequenced directly. In some embodiments, sequencing adapters may be added by PCR amplification, followed by clustering and sequencing.

FIG. 15C is a schematic diagram showing the addition of sequencing adapters to a nanoball concatemer for subsequent clustering and sequencing. The PCR reaction may use a pair of amplification primers 1532 and 1538. Amplification primer 1532 may include a sequencing adapter sequence 1534 (e.g., a P7 adapter sequence) and an index sequence 1536 (e.g., a sample index sequence). Amplification primer 1538 may include a second sequencing adapter sequence (e.g., a P5 adapter sequence). Amplification primers 1532 and 1538 are used in the PCR reaction to initiate amplification of nanoball 1530 to generate multiple single probe copies 1540 of modified probe 1525 that now include the adapter sequences and the index sequence. In this example, a single probe copy 1531 (indicated by the dashed lines) of the sequences in the original circular modified probe 1525 is shown. A bridge amplification reaction may then be performed to generate a clonal cluster 1540 for sequencing. Sequencing may be performed as a single read (A) or as multiple reads (B). Sequencing as a single read provides the UMI sequence, the code sequence, and the index sequence. Sequencing as multiple reads may include, for example, one read to provide the UMI and code sequences, and a second read to provide the index sequence.

In another embodiment, the probes of the invention may include restriction sites. The probes may be designed with restriction sites, or the restriction sites may be added to the probes as part of the assay process. The restriction sites will be amplified into the nanoball and will provide multiple sites at which to cut the nanoball into fragments.

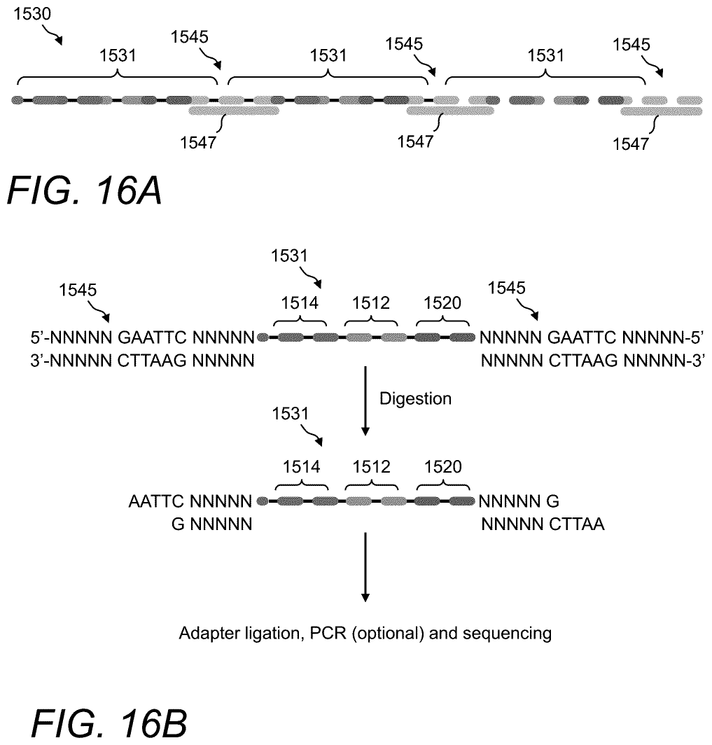

FIG. 16 is a schematic diagram of an example of a portion of nanoball 1530 of FIG. 15 that includes restriction sites that may be used to separate repeated copies of the probe in the nanoball. Referring to panel “A”, in this example, nanoball 1530 includes three probe copies 1531 that may be separated by cleavage at a restriction endonuclease site 1545. A restriction site (RS) complementary sequence 1547 may be hybridized to restriction sites 1545 to provide a double-stranded region for cleavage.

Referring to panel “B”, restriction sites consist of a recognition sequence and flanking bases to ensure that strands remain hybridized after cleavage. Flanking sequences (NNNNNN) may be of length ranging from about 5 to about 50 bases and can be designed to minimize interactions with other probe components and tune the melting temperature (Tm). In this example, the flanking sequences include five bases (N). The RS sequences can be used as an SBS primer such that sequencing begins with the code or may include a spacer region that is read prior to the code.

Digestion of nanoball 1530 hybridized to RS complementary sequences 1547 yields many code-containing DNA fragments with termini that contain single-stranded DNA overhangs or “sticky ends”. The digestion products may be further processed for sequencing. For example, adapters may be ligated to the sticky ends resulting from the restriction digestion.

Alternatively, the ends may be blunt ended (i.e., the single-stranded overhangs removed) and prepared for ligation to adapters. Blunt ended fragments may then be processed via typical sequencing sample preparation protocols such as A-tailing and adapter ligation.

An additional embodiment includes using a primer and polymerase to create RCA products where the entire concatemer is double stranded. This structure can then be processed via the restriction endonuclease procedure described above.

Another embodiment includes employing hyperbranched RCA to create many double stranded, code-containing sequences that can be processed via the restriction endonuclease procedure described above.

In certain embodiments, the restriction endonuclease may be a member of the cas family of proteins or a derivative thereof. These proteins recognize longer sequences of DNA, making them more specific.

In an additional embodiment, circularized probes may be prepared for sequencing without RCA.

In certain embodiments, the nanoballs of the invention may be compacted prior to sequencing. Rolling circle amplification produces linear concatemers of single-stranded DNA.

When the substrate for RCA is a circularized probe, these concatemers may contain 100s-1000s of copies of a code. When preparing RCA products for sequencing, it is useful to compact them. The compacting may produce spherical structures. The compacted structures can increase localization of signal.

Compaction of RCA products into spherical nanoballs can be accomplished by a variety of techniques. In one embodiment, cationic additives that condense high molecular weight DNA (e.g., spermidine, Mg ions, cationic polymers) may be used. The compactness of a spherical nanoball may be tuned by controlling the concentration of the cationic reagent used. The concentration of the cationic reagent used may be selected to avoid aggregation of multiple nanoballs.

In one embodiment, multivalent oligonucleotide sequences that crosslink sites on RCA products may be used to compact RCA products into spherical nanoballs. The RCA binding sites may be separated by a nucleic acid or polymeric linker to control the degree of compaction. The compactness of the spherical nanoball may, for example, be tuned by controlling the degree of crosslinking in the RCA product.

In one embodiment, incorporation of modified nucleotides followed by crosslinking may be used to compact RCA products into spherical nanoballs. Examples of modified nucleotides that may be used include biotinylated nucleotides that bind to streptavidin proteins and nucleotides that covalently react with multifunctional linkers (e.g., amino nucleotides and NHS-terminated linkers). The compactness of the spherical nanoball may, for example, be tuned by controlling the degree of crosslinking in the RCA product.

In certain embodiments, the assays of the invention make use of nanopore sequencing. A nanoball or a circular modified probe may be sequenced using nanopore sequencing. Various nanopore sequencing sample preparation techniques are known in the art. Amplification is optional. Various components required for other sequencing techniques, such as sequencing primers, may be omitted from the probe. Purification can be accomplished using, for example, SPRI beads or BluePippen. Oxford Nanopore Technologies, Inc. (Oxford, UK) provides kits for sample preparation. Examples include Ligation Sequencing Kit, Native Barcoding Kit 96, and Rapid Barcoding Kit.

In certain embodiments, it may be useful to further amplify RCA products prior to sequencing. For example, in applications that use cell-free DNA (cfDNA) as the input where the analyte number may be low, it may be useful to amplify the RCA product prior to sequencing. In one embodiment, a circle-to-circle amplification approach may be used to produce multiple RCA products from one initial RCA product by monomerization of the concatemer (i.e., cleavage to unit length fragments), recircularization of the unit length fragments (i.e., monomers) and amplification of the newly generated circles in a second RCA reaction to produce multiple RCA product copies for further processing or sequencing. The restriction enzyme approach described with reference to FIG. 16 may be used to digest the initial RCA product to unit length (i.e., monomers). In some cases, an end-to-end joining oligonucleotide plus an end-to-end ligation reaction may be used to circularize the unit size fragments.

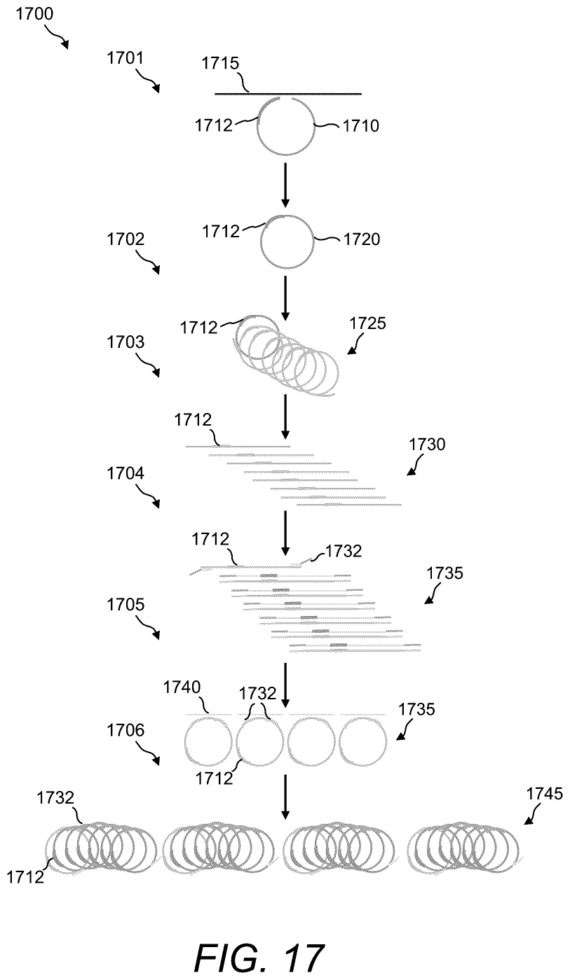

FIG. 17 is a schematic diagram of an example of a process 1700 for circularizing and amplifying unit length nanoball fragments to produce multiple RCA nanoball products. Workflow 1700 may include, but is not limited to, the following steps.

In a step 1701, a probe is hybridized to a target and circularized to yield a circular modified probe. For example, a probe 1710 that includes a code 1712 and a restriction site (not shown) is hybridized to target 1715. A ligation reaction is then performed to circularize probe 1710 to produce a circular modified probe 1720.

In a step 1702, the circular modified probe 1720 is amplified in an RCA reaction to generate a nanoball product 1725. During amplification, the restriction site is amplified into the nanoball and provides multiple sites at which to cut nanoball 1725 into fragments.

In a step 1703, the nanoball product is cleaved to produce multiple unit sized fragments each comprising the code. For example, nanoball 1725 is cleaved at the restriction sites to produce multiple unit size fragments 1730 each comprising code 1712. The cleavage reaction may, for example, be performed as describe with reference to FIG. 16.

In a step 1704, the unit size fragments are amplified in a PCR reaction to generate multiple double-stranded fragments. For example, indexed amplification primers 1732 are hybridized to unit size fragments 1730 and a PCR reaction is performed to produce multiple unit size fragments 1735 that include code 1712 and the indexed amplification primer 1732.

In a step 1705, the amplified unit size fragments are circularized to generate circular unit size fragments. For example, an end-to-end joining oligonucleotide 1740 that is complementary to sequences in amplification primer 1732 is hybridized to unit size fragment 1730 and an end-to-end ligation reaction is performed to generate circular unit size fragments 1735 comprising the code.

In a step 1706, the circular unit size fragments are amplified in a second RCA reaction to produce multiple nanoball copies for further processing or sequencing. For example, circular unit size fragments 1735 are amplified in an RCA reaction to produce multiple nanoballs 1745 each comprising code 1712 and indexed amplification primers 1732.

In an embodiment of process 1700 of FIG. 17, the PCR amplification step 1704 may be omitted and the unit size fragments comprising the code may be re-circularized for subsequent amplification in a second RCA reaction.

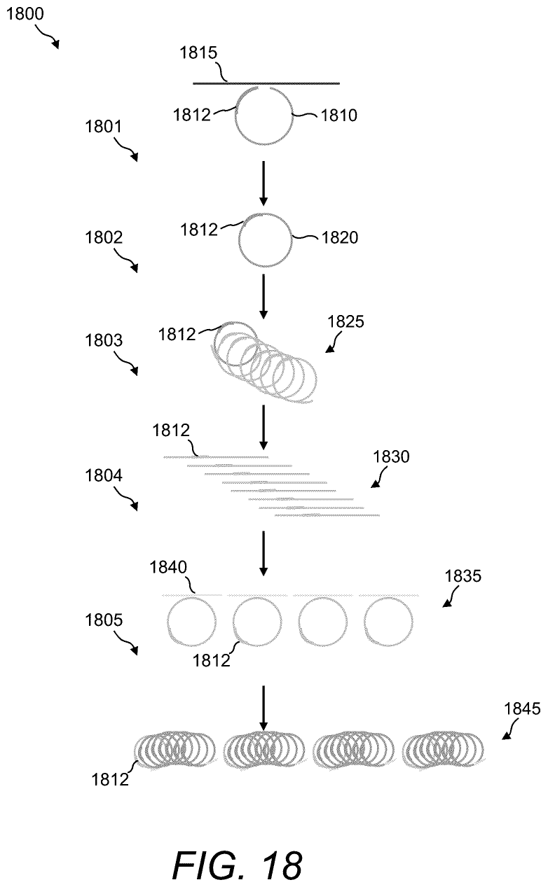

FIG. 18 is a schematic diagram of an example of an alternative process 1800 for circularizing and amplifying unit length nanoball fragments to produce multiple RCA nanoball products. Workflow 1800 may include, but is not limited to, the following steps.

In a step 1801, a probe is hybridized to a target and circularized to yield a circular modified probe. For example, a probe 1810 that includes target recognition sequences (not shown), a code 1812 and a restriction site (not shown) is hybridized to a target 1715. A ligation reaction is then performed to circularize probe 1810 to produce a circular modified probe 1820.

In a step 1802, the circular modified probe 1820 is amplified in an RCA reaction to generate a nanoball product 1825. During amplification, the restriction site is amplified into the nanoball and provides multiple sites at which to cut nanoball 1825 into fragments.

In a step 1803, the nanoball product is cleaved to produce multiple unit sized fragments each comprising the code. For example, nanoball 1825 is cleaved at the restriction sites to produce multiple unit size fragments 1830 each comprising code 1812. The cleavage reaction may, for example, be performed as describe with reference to FIG. 16.

In a step 1804, the unit size fragments are circularized to generate circular unit size fragments. For example, a splint oligonucleotide 1840 that is complementary to the target recognition sequences in unit size fragments 1830 is hybridized to the fragments and a ligation reaction is performed to generate circular unit size fragments 1835 comprising the code.

In a step 1805, the circular unit size fragments are amplified in a second RCA reaction to produce multiple nanoball copies for further processing or sequencing. For example, circular unit size fragments 1835 are amplified in an RCA reaction to produce multiple nanoballs 1845 each comprising code 1812.

Examples of sequencing techniques suitable for use with the assays disclosed herein include nanopore sequencing, next-generation sequencing, massively parallel sequencing, Sanger sequencing, sequencing by synthesis (SBS), pyrosequencing, sequencing by hybridization, single molecule real-time sequencing, SOLID, and sequencing by ligation.

In some embodiments, a process for circularizing a probe may include a gap-fill ligation reaction that may be used to circularize the probe and capture an unknown region of the target that may then be sequenced along with the code.

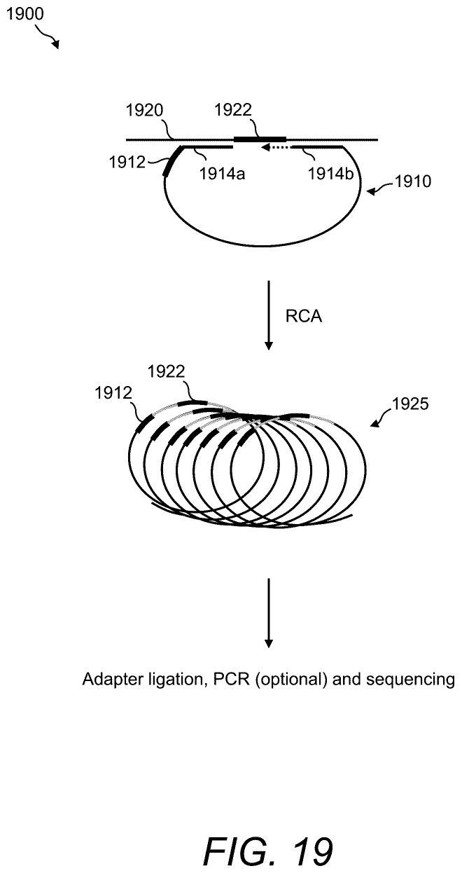

In some embodiments, an unknown region of a target sequence may be captured by a probe transformation reaction and sequenced along with the code. FIG. 19 is a schematic diagram of an example of a process 1900 for capturing an unknown region of a target for sequencing. Process 1900 may include, but is not limited to, the following steps.

In a step 1910, a probe is hybridized to a target and circularized in a gap-fill ligation reaction that captures an unknown region of the target sequence. For example, a probe 1910 that includes a code 1912 (among other elements not shown) and a pair of target recognition elements 1914 (e.g., 1914a and 1914b) is hybridized to a target analyte 1920. Target 1920 may include a region 1922 comprising an unknown sequence. Target recognition elements 1914a and 1914b recognize and bind to target 1920 at sites flanking region 1922. A gap-fill ligation reaction (indicated by dashed arrow) is performed to copy region 1922 into probe 1910 and circularize the probe to yield a circular modified probe (not shown) comprising the unknown region of target 1920. The ligation reaction may be followed by an exonuclease digestion step to remove unligated probes 1940 and target.

In a step 1915, the circular modified probe is amplified in an RCA reaction to form an RCA product 1925 comprising multiple copies of the unknown region 1922 and the code 1912 (among other sequences). The RCA product 1925 may be sequenced directly or sequencing adapter may be added by PCR amplification, followed by clustering and sequencing as described herein above.

Methylation Assay Workflows

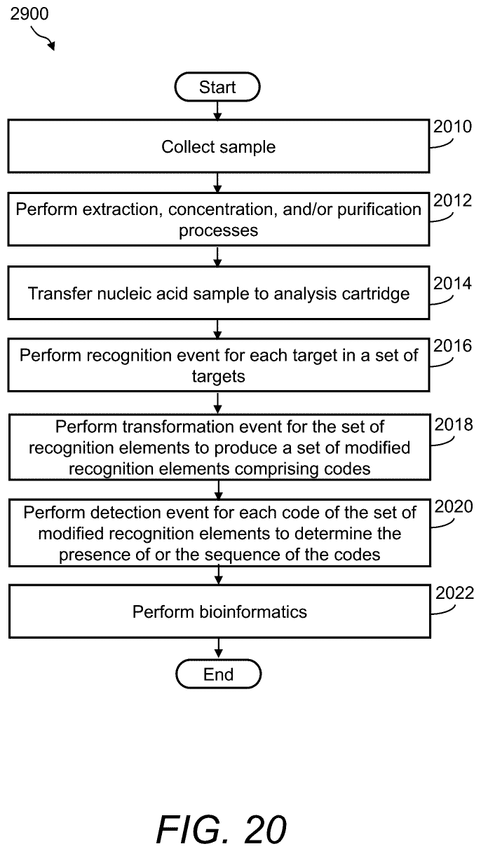

FIG. 20 is a flow diagram of an example of a methylation assay workflow 2000. Methylation assay workflow 2000 may include, but is not limited to, the following steps.

At a step 2010, a sample is collected. For example, a blood or saliva sample may be collected. In one example, a whole blood sample may be collected and processed to separate the plasma fraction from the cellular components of whole blood.

At a step 2012, nucleic acid extraction, concentration, and/or purification processes are performed. For example, the cell-free DNA (cfDNA) in a plasma sample may be extracted, purified, and concentrated for methylation analysis. For example, a proteinase K (ThermoFisher, Waltham, MA) digestion step may be used to digest proteins present in the plasma sample. In some cases, a heat denaturation step (e.g., 94-98° C. for 20-30 seconds) may be used to denature double-stranded DNA into single-stranded nucleic acid. A bead-based extraction and concentration protocol may be used to capture single-stranded DNA in the plasma sample. In some embodiments, the bead-based extraction protocol uses magnetically responsive nucleic acid capture beads. The bead-bound DNA may be released from the capture beads using an elution buffer (or other elution means suitable to the capture bead used) to produce a processed DNA sample for analysis.

At a step 2014, the processed DNA sample is transferred into the analysis cartridge. In one example, the analysis cartridge includes an array of nanowells.

At a step 2016, a recognition event for each target in a set of targets is performed. For example, each target is uniquely recognized by and bound to a recognition element associated with a code, e.g., a trellis code (and optionally other elements). In one example, the recognition event for the set of targets uses a panel of coded padlock probes. The recognition event yields a set of coded targets comprising the target and the recognition element.

At a step 2018, a transformation event for the set of recognition elements is performed to produce a set of modified recognition elements comprising codes. For example, the coded padlock probes may be circularized (i.e., ligated together) in the transformation process if a target matching the probe sequence is present in the sample, thereby generating a readable code sequence. A DNA sample may undergo a variation of this reaction in which the ligation efficiency is affected by the methylation status of the locus being interrogated. Methylation specificity may be provided by several different methods including, for example: 1) methylation-specific inhibition of ligation, 2) methylation-specific aptamers, 3) methylation-specific restriction enzymes or 4) methylation-specific immuno-precipitation. The use of coded padlock probes for detecting targets of interest is described in more detail below with reference to FIG. 5 through 10.

At a step 2020, a detection event is performed for each code of the set of modified recognition elements to decode the codes. In some embodiments, the detection event may include an amplification step in which the code sequence (among other elements) is amplified. In one embodiment, the amplification step comprises a rolling circle amplification reaction (RCA) to generate a nanoball output product. In another example, the amplification step comprises a linear PCR amplification reaction to generate double stranded DNA amplicons. The amplified code may be decoded to associate the code with the target nucleic acid. In one example, the amplified code may be sequenced to determine the sequence of the code associated with the target nucleic acid. In another example, the code may be decoded using a hybridization-based detection process using, for example, fluorescent oligonucleotide probes.

At a step 2022, using the decoded code information from step 2020, bioinformatics may be performed.

Methylation-Specific Inhibition of Encoded Probe Ligation

In one embodiment of workflow 2000, a methylated target sequence may be detected using a methylation-specific inhibition of ligation reaction. Methylation specific inhibition of ligation may be based on the use of a probe or protein that binds specifically to methylated nucleic acid groups thereby interfering with the activity of the ligase and preventing the circularization of the coded padlock probes. For example, if an antibody or methyl CpG binding domain (MBD) or the Uhrflpr domain is bound to the methyl-C targeted by the coded padlock probe it effectively prevents ligation of the probe. When compared to the amount of probe ligated in the reference sample the relative fraction of methylation at the specific locus can be determined.

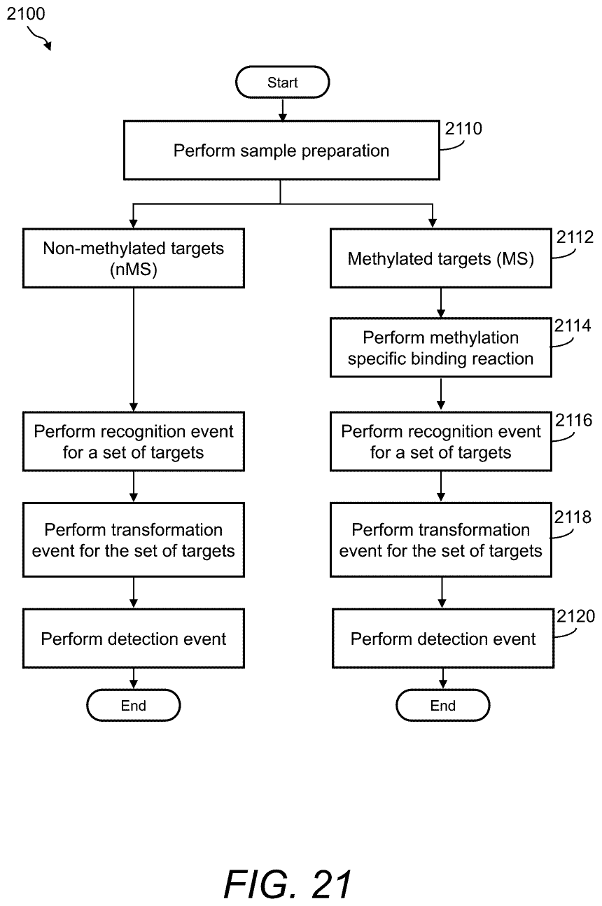

FIG. 21 is a flow diagram of an example of a methylation assay workflow 2100 of direct detection of methylated nucleic acids using methylation-specific inhibition of ligation. Methylation assay workflow 2100 may include, but is not limited to, the following steps.

In a step 2110, a sample preparation process is performed. In one example, the process preparation steps may include step 2010 through step 2014 described with reference to FIG. 20.

In a step 2112, the processed DNA sample is divided into two parallel streams for analysis. For example, the processed DNA may be separated into two parallel streams for analysis-one for methylation sensitive or methylation specific analysis (MS) and one for non-methylation sensitive or specific analysis (nMS). The MS sample may be used to determine the methylation status of certain loci within the nucleic acid and the nMS sample may serve as a baseline reference enabling the fraction or percent of methylation to be determined at each locus of interest (e.g., about 20% or less, or about 10% or less,). The sample may be split evenly between the MS and nMS analyses or the sample may be unevenly divided between the two types of analyses.

At a step 2114, a methylation specific binding reaction is performed on the MS sample. For example, a methylation specific binding protein is combined with the MS sample and incubated for a period of time sufficient for protein binding to methylated cytosines. Examples of methylation specific binding proteins that may be used include monoclonal antibodies, polyclonal antibodies, nanobodies, MBD or Uhrf1pr proteins.

At a step 2116, a recognition event for each target in a set of targets is performed for both the MS and nMS samples. For example, each target is uniquely recognized by and bound to a recognition element associated with a code. In one example, the recognition event may use a panel of coded padlock probes as described in step 2016 of FIG. 20. The coded padlock probes may include universal amplification primer sequences. In one embodiment, the coded padlock probes may include a non-extendable or blocking sequence located between a pair of amplification primer sequences. Incorporation of a non-extendable sequence into the encoded probe may prevent generic rolling circle amplification (RCA) of the padlock probe, thereby allowing for linear double-stranded PCR products. The recognition event yields a set of coded targets comprising the target and the recognition element.

At a step 2118, a transformation event for the set of recognition elements is performed. For example, the coded padlock probes may be circularized (i.e., ligated together) in the transformation process if a target matching the probe sequence is present in the sample, thereby generating a readable code sequence. In the MS sample, the coded padlock probes may anneal to the target sequence and ligation may occur if the target sequence is not methylated. If the target sequence is methylated, the binding proteins bound to the methylated nucleotides in the MS sample effectively interfere with binding and ligation of the padlock probe.

At a step 2120, a detection event is performed. For example, a detection event for each code of the set of modified recognition elements is performed to determine the sequence of the codes. The detection event may include an amplification step in which the code sequence (among other elements) is amplified. In one embodiment, the amplification step comprises a linear PCR amplification reaction to generate double-stranded DNA amplicons. In one embodiment, the amplification step comprises an RCA reaction. In the amplification step, only the unmethylated target sequences in the MS sample may be amplified because the methylated sequences were blocked from coded padlock probe ligation by the methylation specific proteins or antibodies. In the nMS sample all or substantially all of the target sequences may be amplified because of the absence of any methylated specific binding proteins. The amplified code may be decoded to associate the code with the target nucleic acid. In one example, the amplified code may be sequenced to determine the sequence of the code associated with the target nucleic acid.

In an embodiment of workflow 2100, methylation-specific aptamers may be used for the indirect detection of 5-methylcytosine (5-mC) found in CpG dinucleotide motifs. Aptamers may be created that bind specifically to 5-mC and thus may be used to elucidate the state of methylation of CpGs. The binding specificity is independent of the 5′-and 3′-NA sequence flanking the CpG dinucleotide with the exception of when CpGs are in close proximity with one another. Because of their smaller footprint relative to antibodies and methyl domain binding proteins, aptamers may be used to improve the resolution of detecting clusters of methylated sites in close proximity. Aptamers can be created to bind to a single 5-mCpG or to two or more adjacent 5-mCpGs or to multiple 5-mCpG that have one or more nucleotides inserted between the 5-mCpGs. A collection of aptamers can be employed to differentiate methylated and non-methylated CpGs that are found alone or near other CpGs.

The aptamers may be used in combination with coded padlock probes or restriction endonucleases. Coded padlock probes may be designed to hybridize to the nucleic acid sequences flanking the methylated or unmethylated CpG. In combination with aptamers, the presence or absence of coded padlock probe hybridization may provide a mechanism to determine the methylation status of specific 5-mCs. If an aptamer is bound to a 5-mC, it may prevent the annealing of the coded padlock probe to that region. If the coded padlock probe is unable to anneal to the target nucleic acid sequence, ligation of the probe will not occur leading to no signal for that specific 5-mC. If the CpG dinucleotide is unmethylated, the coded padlock probe can hybridize to the nucleic acid target sequence and ligation can occur producing a positive signal.

### Coded Padlock Probe Ligation in Combination With a Methylation-Specific Restriction Enzyme

In another embodiment of workflow 2000, a methylated target sequence may be detected using a padlock probe ligation reaction in combination with a methylation-specific restriction enzyme digestion reaction. For example, a methylation sensitive endonuclease such as Hhal may be used to cut a coded padlock probe-nucleic acid fragment resulting in the inability to produce a rolling circle amplification (RCA) or PCR product. If the target nucleic acid is methylated, Hhal is unable to cut the fragment which may then be subsequently amplified and detected by PCR or other methods. Examples of methylation-specific restriction endonucleases that may be used include, but are not limited to, Aat II, Acc II, Aor13H I, Aor51H I, BspT104 I, BssH II, Cfr10 I, Cla I, Cpo I, Eco52 I, Hae II, Hap II, Hha I, Mlu I, Nae I, Not I, Nru I, Nsb I, PmaC I, Psp 1406 I, Pvu I, Sac II, Sal I, Sma I, and SnaB I.

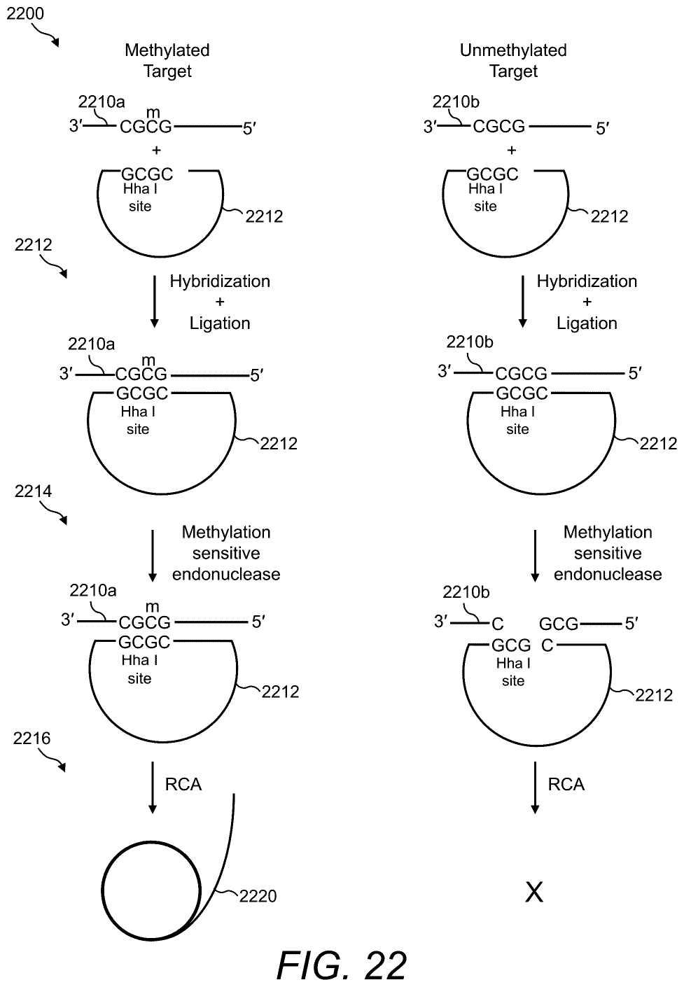

FIG. 22 is a schematic diagram illustrating a process 2200 for detecting methylated nucleic acids using padlock probe ligation in combination with methylation-specific restriction enzyme digestion. Sample preparation for input into process 2200 may, for example, be performed as described for FIG. 20 starting from a whole blood sample (step 2010), performing the nucleic acid extraction, concentration, and/or purification processes (step 2012), and transferring the nucleic acid sample to the analysis cartridge (step 2014). In this example, the nucleic acid sample may include a target sequence of interest, where the target sequence may be a methylated target 2210a or an unmethylated target 2210b. In FIG. 22, two different schemes (i.e., “Methylated Target” and “Unmethylated Target”) are shown to highlight the different outcomes for detecting a methylated target versus an unmethylated target, however both reactions may occur in a single sample and only the methylated targets are detected. Process 2200 may include, but is not limited to, the following steps.

In step 2212, a recognition event and a transformation event are performed. In the recognition event, a target is recognized and bound by a recognition element associated with a code. In this example, the recognition element is coded padlock probe. For example, hybridization probes 2212 that are designed to hybridize adjacent to a target site of interest are hybridized to the methylated target 2210a and the unmethylated target 2210b. Hybridization probe 2212 includes, for example, a primer sequence (not shown), a sample index (not shown), a code element (not shown), and sequences for a methylation sensitive endonuclease site Hha I that flank a target site of interest.

In the transformation event, a ligation reaction is performed to ligate the target bound recognition elements (i.e., hybridization probes 2212) comprising the code. Ligation occurs for both the methylated target 2210a and unmethylated target 2210b.

In step 2214, a methylation sensitive endonuclease digestion reaction is performed. For example, the methylation sensitive endonuclease will cut at an unmethylated site but will not cut at a methylated site. In this example, the methylation sensitive endonuclease is Hha I. In the presence of Hha I the methylated target 2210a is not cut and the unmethylated target 2210b is cut at the restriction endonuclease site to generate two fragments. In this step, the uncut methylated target 2210a is circularized to create a circular recognition element (not shown) that includes an intact amplification primer site.

In step 2216, an amplification reaction is performed. In this example, the amplification reaction is a rolling circle amplification reaction (RCA) that produces a nanoball product 2220 that includes multiple copies of the target specific code. The uncut methylated target 2210a that is circularized and includes an intact amplification primer site is amplified and the unmethylated target 2210b is not amplified. The amplified code may be identified to associate the code with the target nucleic acid. In one example, the amplified code may be sequenced to determine the sequence of the code associated with the target nucleic acid. In another example, the code may be decoded in a hybridization-based detection process using, for example, fluorescent oligonucleotide probes.

In another embodiment (not shown), hybridization probe 2212 may further include a non-extendable sequence which would allow for linear double stranded PCR products in a subsequent amplification step. In this embodiment, an exonuclease I digestion reaction may then be used to digest any remaining single stranded nucleic acid, such as unreacted coded padlock probes, amplification primers, and single stranded target sequences. This approach may provide a method for differentiating desired products from waste and may allow for an efficient cleanup step.

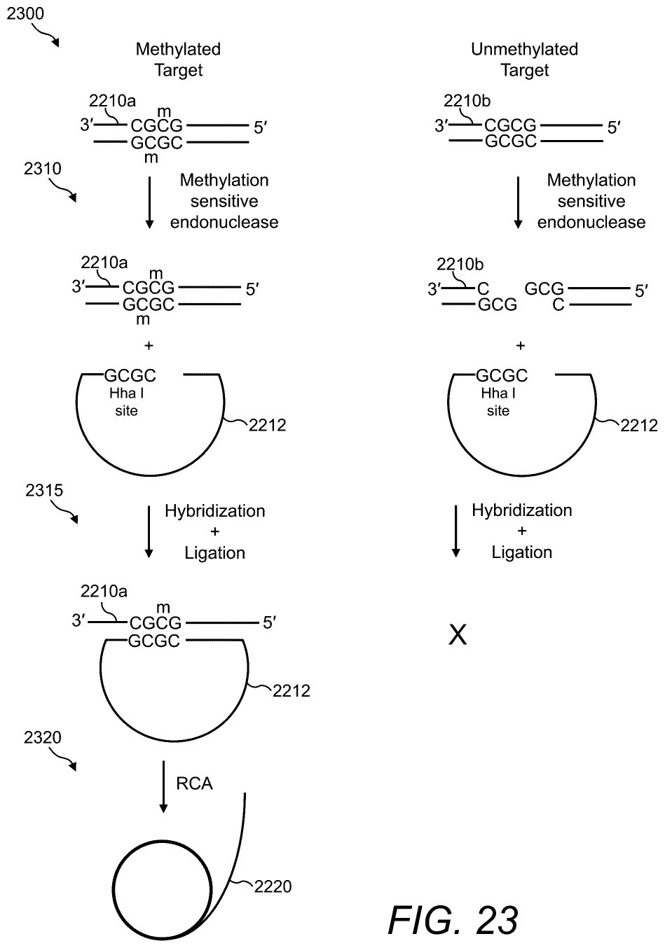

In another embodiment of the example described with reference to FIG. 22, the process steps of hybridization and ligation, and digestion using a methylation sensitive endonuclease may be reversed. FIG. 23 is a schematic diagram illustrating an alternative embodiment of the process described with reference to FIG. 22. In this example, the process step of methylation sensitive endonuclease digestion is performed prior to the step of hybridization and ligation of a target specific padlock probe. In this example, the input sample may be a double-stranded DNA target, such as a cell-free DNA target in a liquid biopsy sample. In FIG. 23, two different schemes (i.e., “Methylated Target” and “Unmethylated Target”) are shown to highlight the different outcomes for detecting a methylated target versus an unmethylated target, however both reactions may occur in a single sample and only the methylated targets are detected.

In step 2310, a methylation sensitive endonuclease digestion reaction is performed. The methylation sensitive endonuclease will cut at an unmethylated site but will not cut at a methylated site. In this example, the methylation sensitive endonuclease is Hha I. For example, in the presence of Hha I the methylated target 2210a is not cut and the unmethylated target 2210b is cut at the restriction endonuclease site to generate two fragments.

In step 2315, a recognition event and a transformation event are performed. In the recognition event, a target is recognized and bound by a recognition element associated with a code, e.g., a trellis code. In the transformation event, a ligation reaction is performed to ligate the target bound recognition element to produce a modified recognition element comprising the code. In this example, the recognition element is a coded padlock probe. For example, hybridization of the uncut methylated target 2210a to padlock probe 2212 and ligation of padlock probe 2212 generates a modified probe that includes an intact amplification primer site (not shown). Hybridization and ligation of the restriction digested fragments of the unmethylated target does not occur and an amplifiable product is not generated.

In step 2320, an amplification reaction is performed. In this example, the amplification reaction is a rolling circle amplification reaction (RCA). Amplification of the modified probe produced by the hybridization and ligation of padlock probe 2212 to the uncut methylated target 2210a produces a nanoball product 2220 that includes multiple copies of the target specific code. An amplification product is not generated from the unmethylated target.

### Dual Encoded Probe Ligation in Combination With a Methylation-Specific Restriction Enzyme

In a variation of the ligation mediated assays/workflows described above for detection of methylated nucleotides in a target of interest, a dual probe approach in combination with a methylation sensitive endonuclease may be used. In this approach, the recognition element comprises a pair of probes (i.e., a dual probe) wherein transformation of the recognition element produces a readable code.

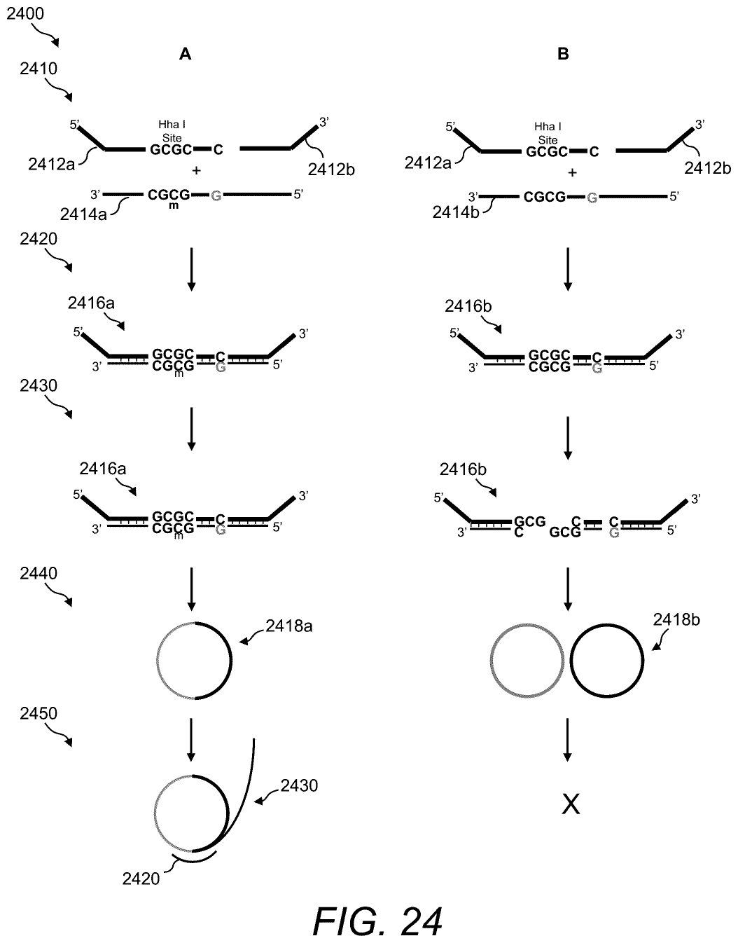

FIG. 24 is a schematic diagram illustrating a process 2400 of using dual hybridization probes in combination with a methylation sensitive endonuclease digestion reaction to differentiate a methylated target sequence and an unmethylated target sequence. Sample preparation for input into process 2400 may, for example, be a performed as described for FIG. 20 starting from a whole blood sample (step 2010), performing the nucleic acid extraction, concentration, and/or purification processes (step 2012), and transferring the nucleic acid sample to the analysis cartridge (step 2014). In this example, the nucleic acid sample may include a target sequence of interest, where the target sequence may be a methylated target 2414a or an unmethylated target 2414b. In FIG. 24, two different schemes (i.e., “A” and “B”) are shown to highlight the different outcomes for detecting a methylated target versus an unmethylated target, however both reactions may occur in a single sample and only the methylated targets are detected. Process 2400 may include, but is not limited to, the following steps and unspecified additional steps.

In a step 2410, a recognition event is performed. For example, a target 2414, (e.g., a cell-free DNA target) is hybridized with a pair of probes 2412a and 2412b. Probes 2412a and 2412b are designed to hybridize adjacent to the target site of interest. One probe in the pair of probes may include a code and one or both probes in the pair of probes may include other functional sequences. For example, probe 2412a may include, a first portion of a primer sequence (not shown), and sequences for a methylation sensitive endonuclease site that flank a target site of interest. Probe 2412b may include a second portion of the primer sequence (not shown). For example, in panel A, a methylated target 2414a is hybridized with probes 2412a and 2412b; and in panel B, an unmethylated target 2414b is hybridized with probes 2412a and 2412b.

In a step 2420, a transformation event is performed to ligate the target bound dual probe recognition element to produce a modified recognition element comprising the code. For example, probes 2412a and 2412b that are hybridized to methylated target 2414a immediately adjacent to each other leaving no gap can be ligated to produce a ligated dual probe 2416a. Similarly, probes 2412a and 2412b that are hybridized to unmethylated target 2414b immediately adjacent to each other leaving no gap can be ligated to produce a ligated dual probe 2416b.

In a step 2430, a methylation sensitive endonuclease digestion reaction is performed to distinguish between the ligated dual probe bound to the methylated target 2416a and the ligated dual probe bound to the unmethylated target 2416b. The methylation sensitive endonuclease will cut at an unmethylated site but will not cut at a methylated site. In this example, the methylation sensitive endonuclease is Hha I. For example, in the presence of Hha I the methylated target 2416a is not cut and the unmethylated target 2416b is cut at the restriction endonuclease site to generate two fragments.

In a step 2440, a single-stranded DNA ligation reaction is performed to generate circularized molecules. The single-stranded ligation creates an intact primer site that can be used in a rolling circle amplification reaction. In one example, the single-stranded DNA ligation reaction is performed using Circligase. In this step, the uncut methylated target 2416a is circularized to create a circular recognition element 2418a that includes an intact RCA primer site. The Hha I cut unmethylated target 2416b is circularized to create two circular modified recognition elements 2418b that do not include an intact RCA primer sites.

In a step 2450, the circularized modified recognition element 2418a is amplified in a rolling circle amplification (RCA) reaction to generate a nanoball product. For example, a primer sequence 2420 that straddles the intact primer site in modified recognition element 2418a may be used to initiate the RCA reaction that generates a nanoball product 2430. Because the primer sequence used in the amplification reaction must straddle an intact primer site, the two circular modified recognition elements 2416b that do not include an intact primer site are not amplified.

In an embodiment of the process described with reference to FIG. 24, the order of performing hybridization and ligation of a target specific padlock probe, and methylation-specific endonuclease digestion may be reversed as described with reference to FIG. 22 and FIG. 23.

### Proximity-Based Ligation Using a Linear Encoded Probe

In some embodiments, a methylation-specific binding protein (e.g., a methylation specific antibody or fragment thereof) may be used in combination with a coded nucleic acid probe for detection of a methylated site in a target sequence of interest. For example, a proximity-based ligation process using a linear detection probe in combination with a modified methyl binding protein (e.g., a methylation specific antibody or fragment thereof) and a location specific oligonucleotide probe may be used for detection of a methylation site in a target sequence of interest.

In some embodiments, the proximity-based ligation process for methylation detection may be used in a solution-phase assay.

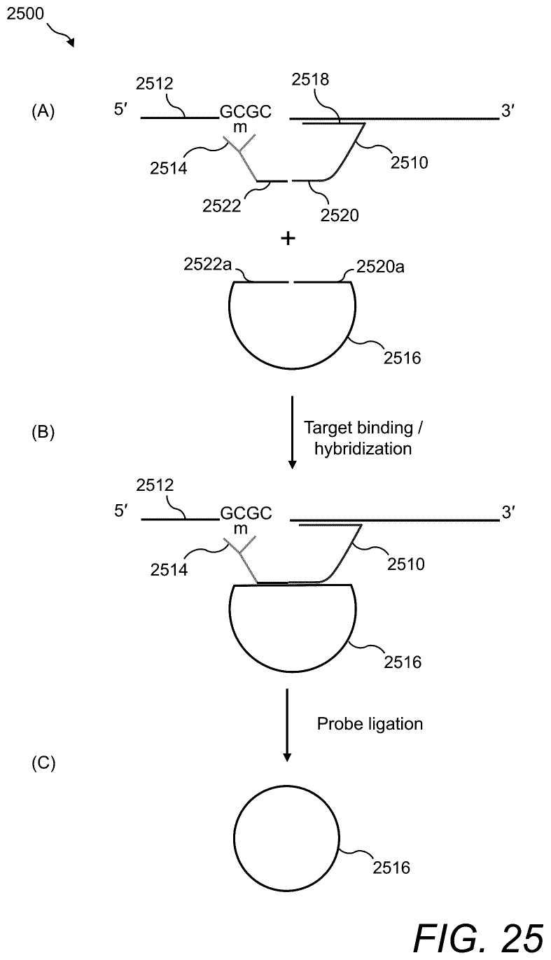

FIG. 25 is a schematic diagram illustrating an example of a solution-phase proximity ligation process 2500 that uses a linear detection probe in combination with a modified methyl binding protein for detection of methylated cytosine. Process 2500 may include, but is not limited to, the following steps as while as other unspecified steps.

In step A, a location-specific oligonucleotide probe 2510 that is designed to hybridize adjacent to a target 2512 is combined in a solution-phase proximity binding reaction with an oligonucleotide-modified methyl binding protein 2514 and a linear detection probe 2516. Location-specific oligonucleotide probe 2510 may include a first hybridization sequence 2518 that is complementary to the target 2512 of interest and a second hybridization sequence 2520 that is complementary to a corresponding sequence 2520a on linear detection probe 2516. Modified methyl binding protein 2514 may be modified with an oligonucleotide 2522 that is complementary to a corresponding sequence 2522a on linear detection probe 2516. Linear detection probe 2516 may further include, for example, a sequences for a split amplification primer binding site (not shown), a sample index (not shown), and a code element (not shown).

In step B, in the presence of target 2512, location-specific oligonucleotide probe 2510 is hybridized to target 2512 via hybridization sequence 2518 and methyl binding protein 2514 binds to the methylated cytosine in target 2512. Hybridization of location-specific oligonucleotide probe 2510 and binding of methyl binding protein 2514 to target 2512 brings hybridization sequence 2520 and hybridization sequence 2522 into proximity of each other. Further, hybridization sequence 2520 and hybridization sequence 2522 may be hybridized to their corresponding sequences 2520a and 2522a, respectively, on linear detection probe 2516, which effectively circularizes detection probe 2516.

In step C, a ligation reaction may then be performed to generate a closed circular detection probe 2516 that now includes an intact primer site that may be used in a subsequent amplification reaction.

In some embodiments, a methylation-specific binding protein (e.g., a methylation specific antibody or fragment thereof) in combination with an encoded detection probe may be used for detection of a methylation site in a target sequence of interest in a solid-phase capture assay. For example, the methylation-specific binding protein may be immobilized on a solid substrate. In one example, the solid substrate is a well of a multi-well plate that includes an array of nanowells. The plurality of immobilized methylation-specific binding proteins may be used to capture a methylated target of interest. In some embodiments, the target of interest includes a fully methylated site. In some embodiments, the target of interest includes a partially methylated site.

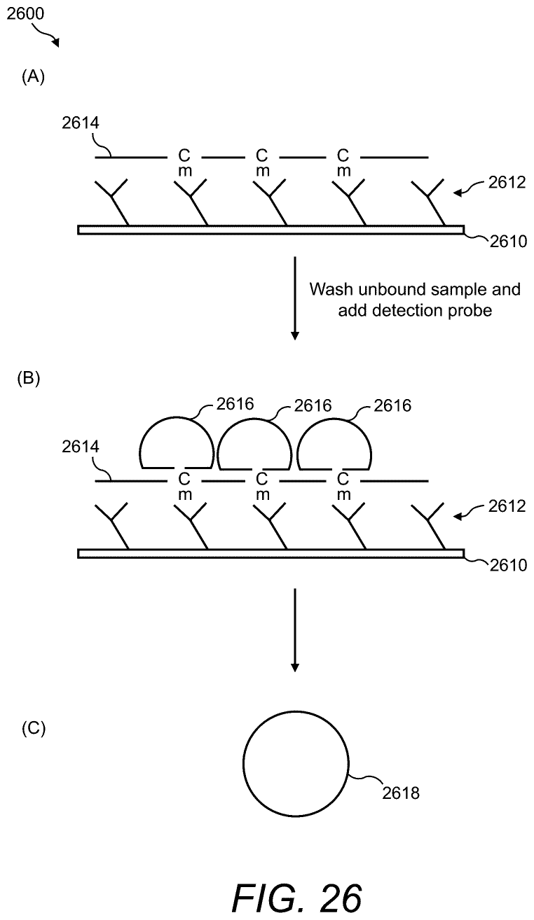

FIG. 26 is a schematic diagram illustrating an example of a solid-phase detection process 2600 for detection of a methylated site in a target sequence of interest. Process 2600 may include, but is not limited to, the following steps as well as other unspecified steps.

In step A, a substrate 2610 that includes a plurality of methyl binding proteins 2612 (e.g., a methylation specific antibody or a fragment thereof) immobilized thereon may be used to capture a target sequence 2614 that includes a plurality of methylated cytosines. In one example, substrate 2610 is a well of a multi-well plate.

In step B, substrate 2610 is washed to remove unbound material and a target-specific detection probe 2616 is added. In one embodiment, target-specific detection probe 2616 may be a target-specific probe that is designed to hybridize to methylated cytosines in a fully or partially methylated target of interest (e.g., CpG islands in a target of interest). Target-specific detection probe 2616 may further include, for example, a sequence for an amplification primer binding site (not shown), a sample index (not shown), and a code element (not shown).

In one embodiment, target-specific detection probe 2616 may be a linear detection probe. In this embodiment, an optional ligation reaction may be performed to circularize the hybridized detection probe prior to a subsequent amplification step. The ligation reaction may be used, for example, to add specificity to the detection reaction as described hereinabove.

In another embodiment, target-specific detection probe 2616 may be a pre-circularized detection probe.

In step C, an amplification reaction is performed. In one embodiment, a rolling circle amplification reaction (RCA) is performed, wherein the hybridized probe (i.e., a circularized probe 2616) is displaced from the templated strand (e.g., target sequence 2614) and amplified into a nanoball 2618 that may be used in a subsequent detection reaction, e.g., a high-throughput sequencing reaction.

### Samples

Examples of tissues from which nucleic acid may extracted using the techniques described herein may include solid tissue, lysed solid tissue, fixed tissue samples, whole blood, plasma, serum, dried blood spots, buccal swabs, other forensic samples, fresh or frozen tissue, biopsy tissue, organ tissue, cultured or harvested cells, and bodily fluids.

In various embodiments, a sample may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes.

### Targets

Targets may include any biological markers. Examples include biological markers for screening or diagnosing cancer. In one embodiment, targets include a panel of methylation markers for diagnosing cancer. Examples of panels of probes which may be targeted are set for the in WO2019195268, entitled “Methylation markers and targeted methylation probe panels,” and WO2020069350A1, entitled “Methylation markers and targeted methylation probe panel,” the entire disclosures of which (including without limitation the sequence listings) are incorporated herein by reference. Targets may be obtained from biopsies, circulating nucleic acid samples, or nucleic acids from other samples.

### Diagnostics and Screening

The methods of the invention may be used for screening or diagnosing a subject for a disease, such as cancer or for selecting a therapy for treating a disease, such as selecting a therapy for treating a cancer.

### Soft Decoding

In the methods of the invention, a soft decoding process may use decoding by hybridization (DBH).

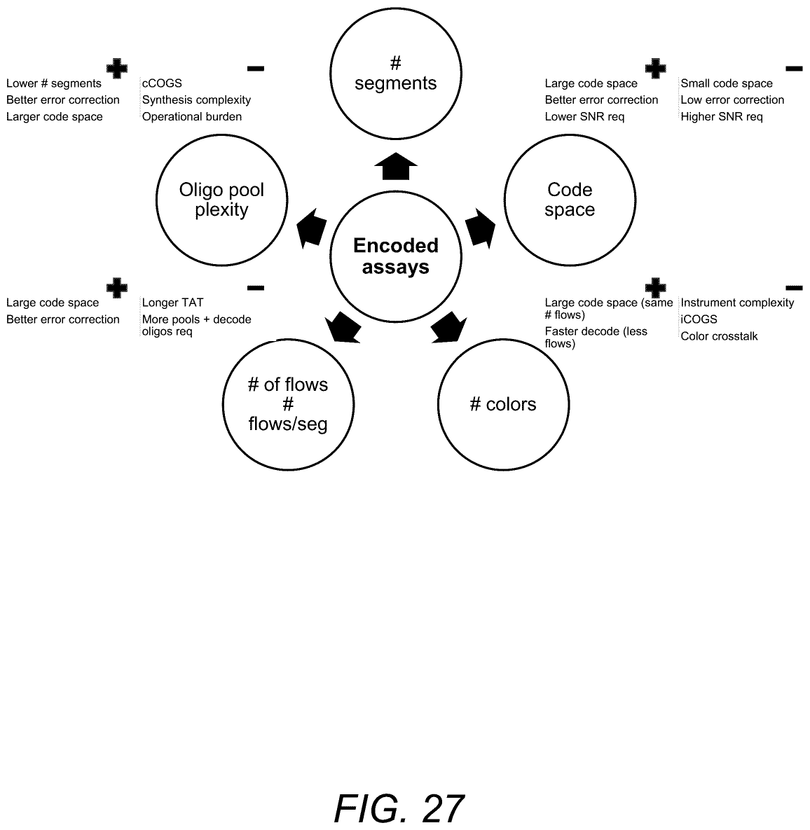

FIG. 27 is a schematic diagram illustrating some of the factors considered in the design of an encoded probe for decoding by hybridization.

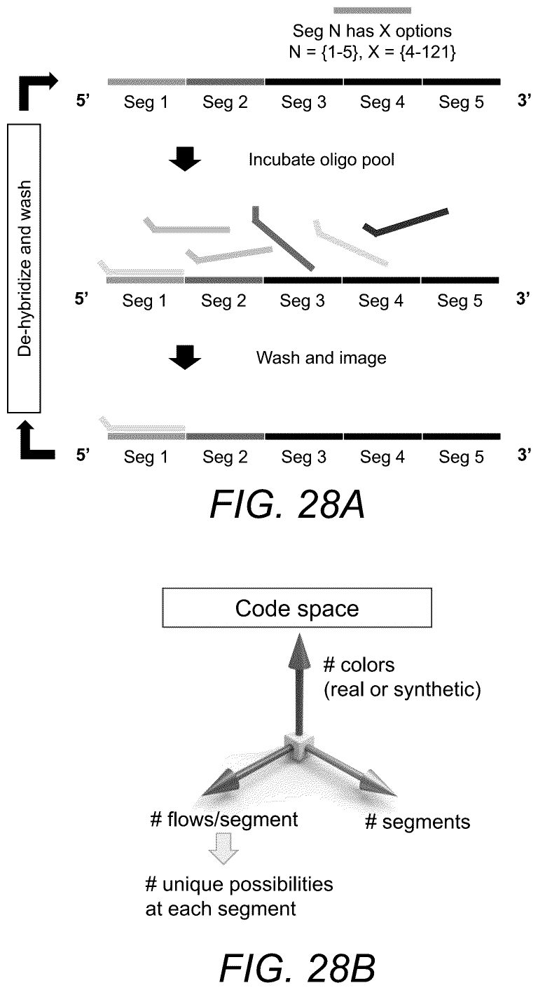

FIG. 28A is a schematic diagram illustrating an overview of process for decoding by hybridization. For example, a code may include 5 segments and decoding may use 1 flow/segment, 4 colors or oligonucleotides in the oligo pool/flow. The decoding by hybridization process may include repeated cycles of hybridizing a code sequence with a decoding oligonucleotide pool (decoding oligos) comprising fluorescently labeled oligos, washing the hybridization reaction to remove unbound decoding oligos, imaging the decoding reaction to determine the identity of the hybridized decoding oligo, and de-hybridizing the code sequence to initiate a subsequent decoding cycle.

FIG. 28B is a schematic diagram illustrating the code space in decoding by hybridization. For example, the code space may include the number of colors (real or synthetic), the number of flows per segment and the number of unique possibilities at each segment, and the number of segments in the code.

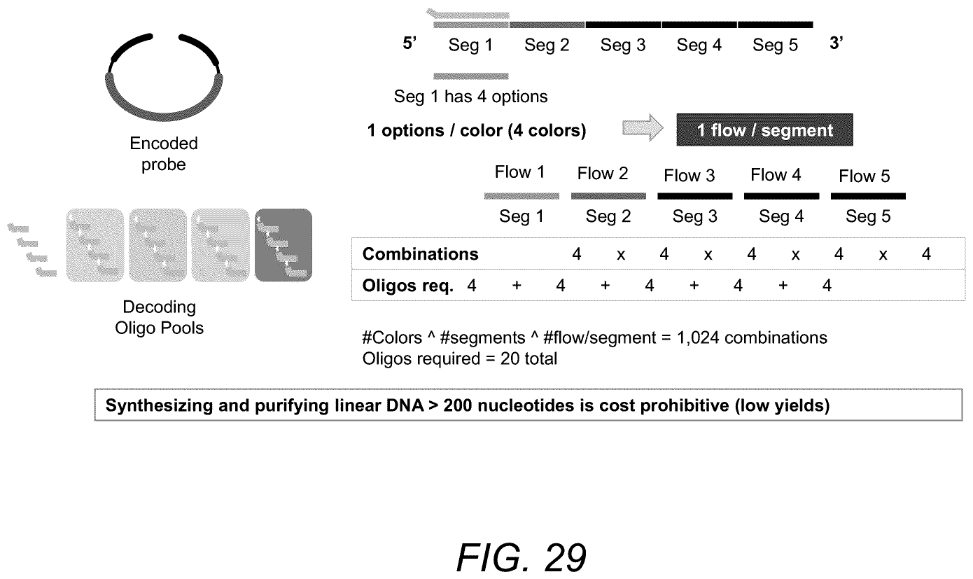

FIG. 29 is a schematic diagram of an example of a method for encoding symbols onto each segment of a code. In this example, the code comprises 5 segments (e.g., seg 1 through seg 5) which requires relatively few decoding oligos for decoding by hybridization. A code with 5 segments would require 5 decoding pools with 4 different labeled decoding oligos flowed for each segment decoded (i.e., 20 different decoding oligos are required).

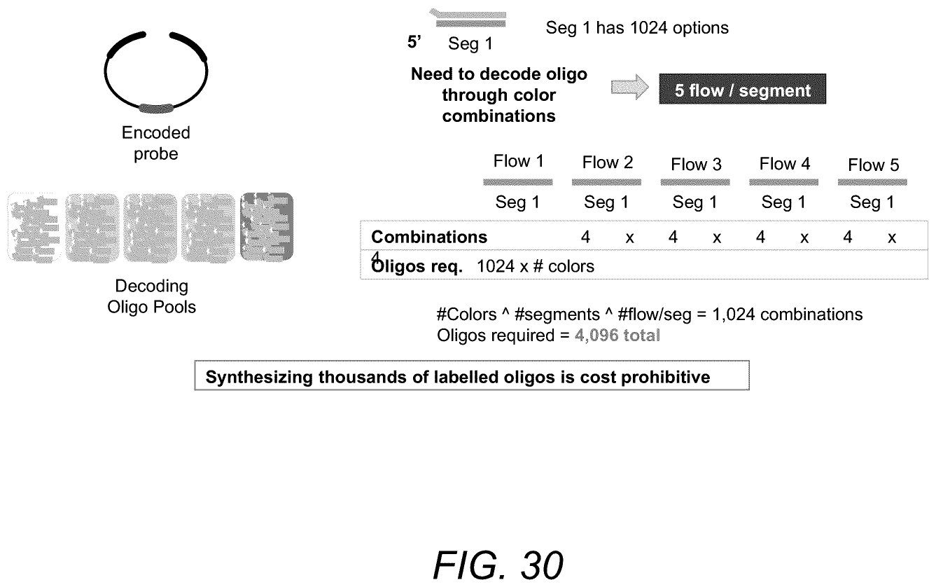

FIG. 30 is a schematic diagram of another example of a method for encoding symbols onto a code wherein the length of the code sequence comprises a single segment that requires a relatively large number of decoding oligos.

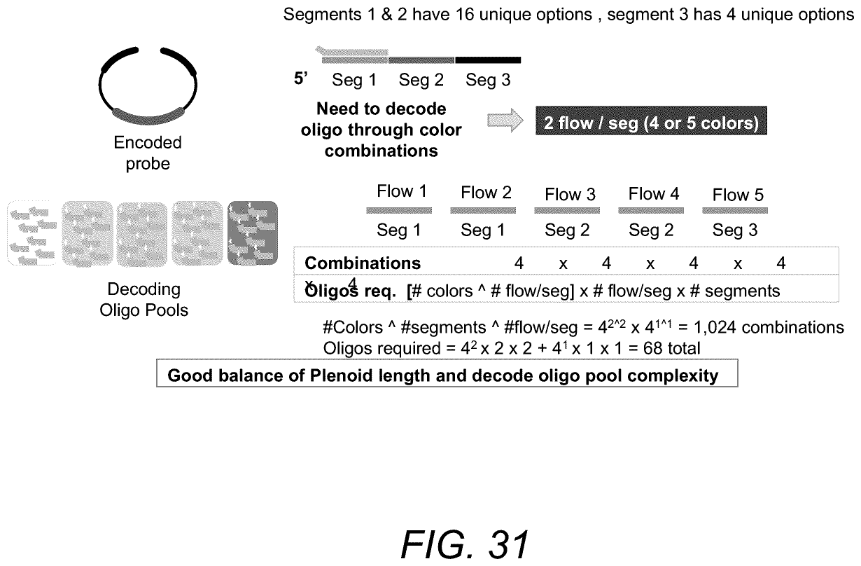

FIG. 31 is a schematic diagram of another example of a method for encoding symbols onto a code wherein the mix of segment number and flows/segment in the decoding process balances the length of a code and the complexity required in the decoding oligo pool.

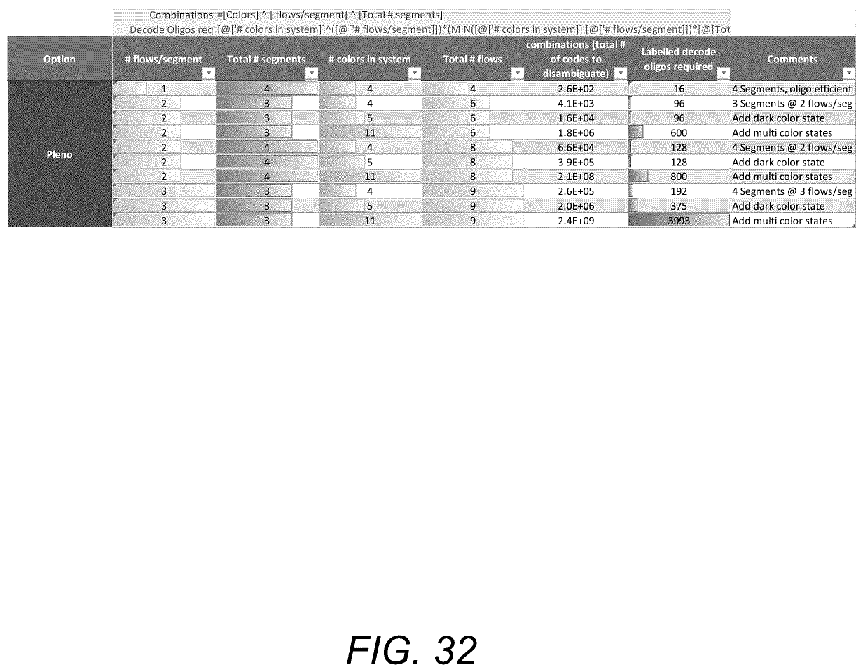

FIG. 32 is a screenshot of an example of the permutations (e.g., colors, flows/segment, total segments, and total flows) that may be used to achieve a relatively large combination space (codes pace) from which select a subset of codes.

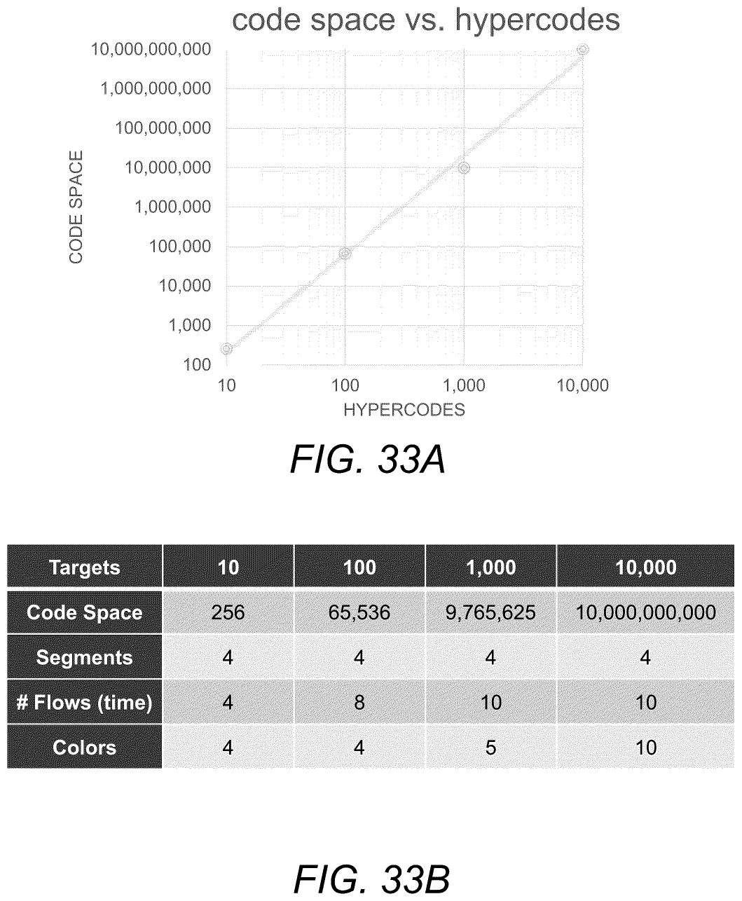

FIG. 33A and FIG. 33B are a plot showing the relationship of the number of codes in a code space, and a summary table of the number of segments, flows, and colors required for a given number of targets for detection, respectively.

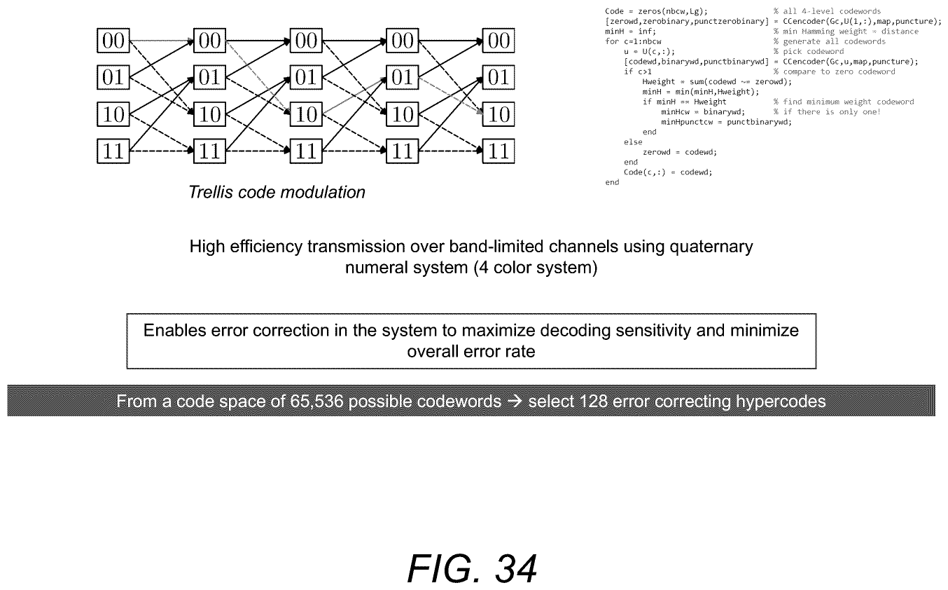

FIG. 34 is a schematic diagram of an example of a trellis code and a process of using the trellis code to select a set of codes with desired properties for an assay from a large code space. In this example, a 4-color system is used, which enables error correction in the system to maximize decoding sensitivity and minimize the overall error rate.

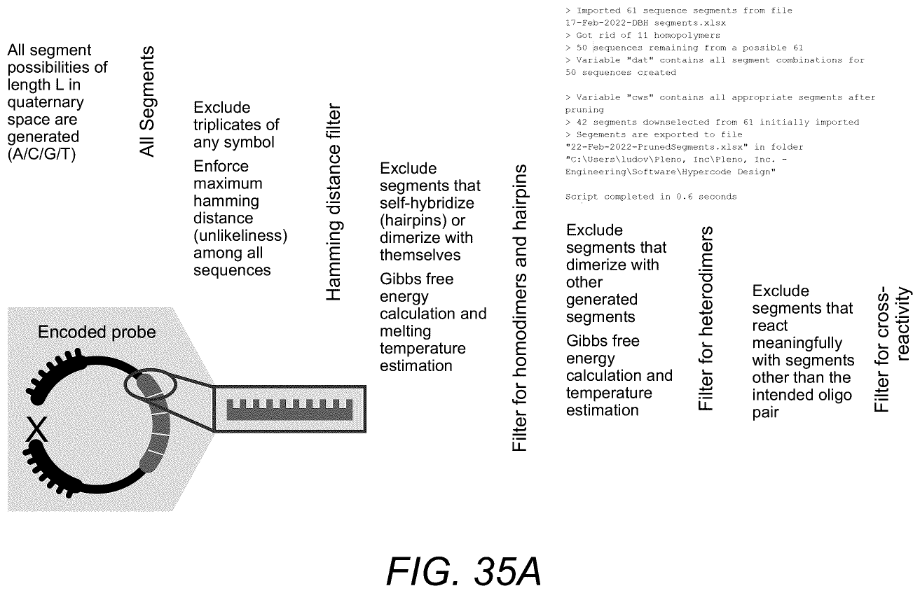

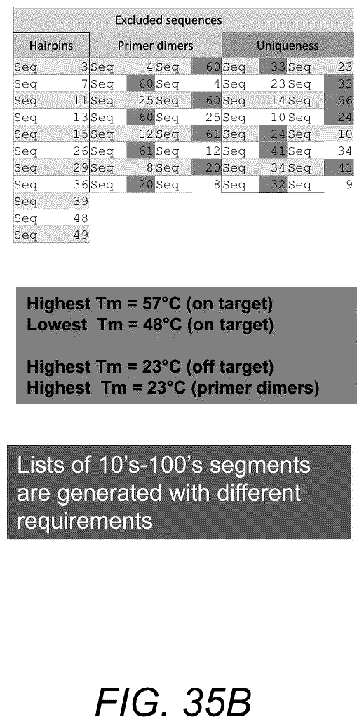

FIG. 35A and FIG. 35B are a representation of a strategy for designing oligo segments on a probe that will encode for the symbols that make up the trellis code (or other). The strategy may include translating the symbol from the code into the DNA backbone of the probe, either through 1 DNA base if sequencing, or decoding by (may be more than 1 base), or many bases if using decoding by hybridization (e.g., between 10-20 bases, though longer and shorter are possible).

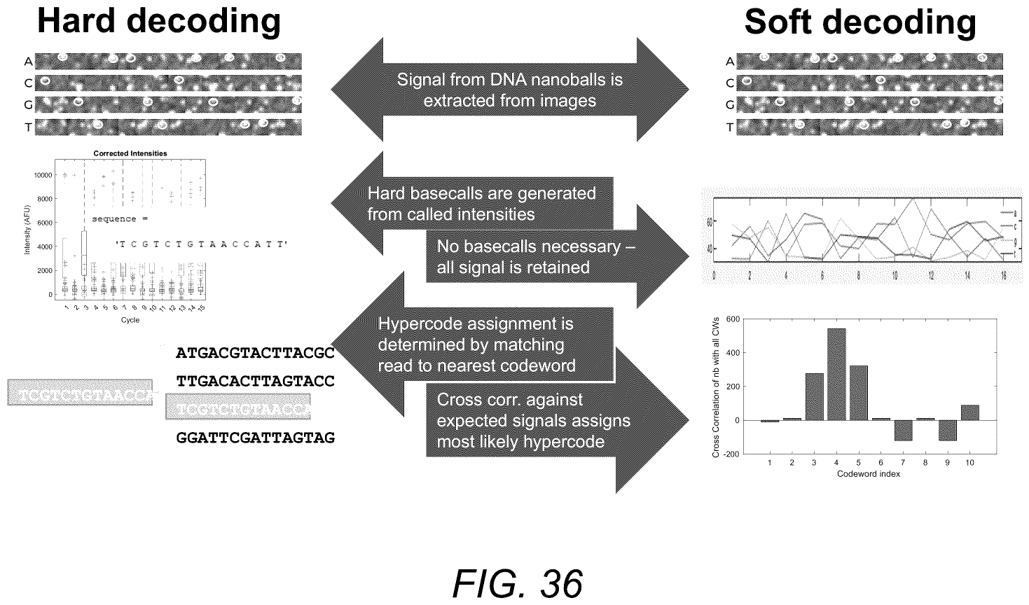

FIG. 36 is a representation of an overview of a decoding process comparing hard decoding vs. soft decoding.

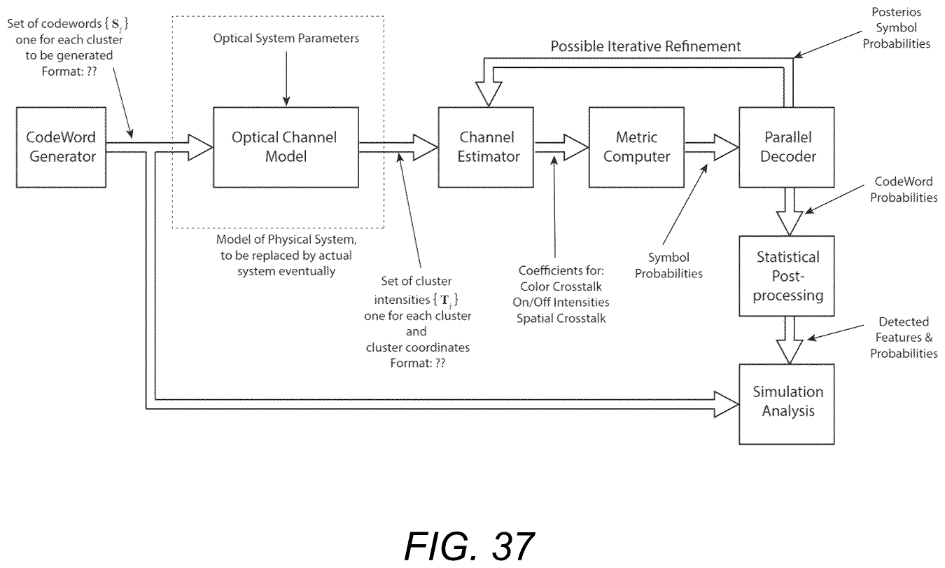

FIG. 37 is a schematic diagram of an example of a soft decoding process that may be used in the assays of the invention.

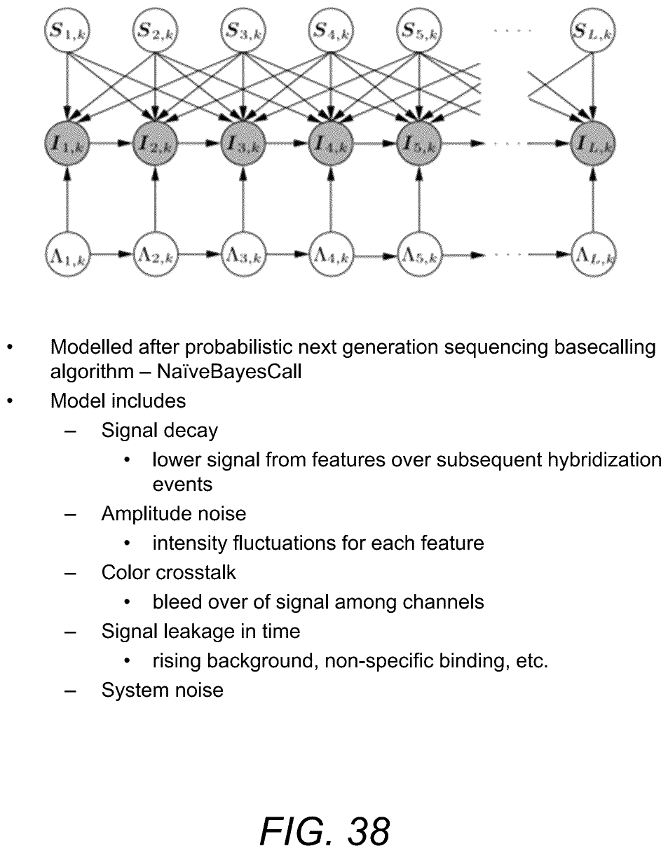

FIG. 38 is a summary of a channel model for a base calling algorithm that may be used in a soft decoding process. The model may include, for example, parameters for signal decay, amplitude noise, color crosstalk, signal leakage in time and system noise.

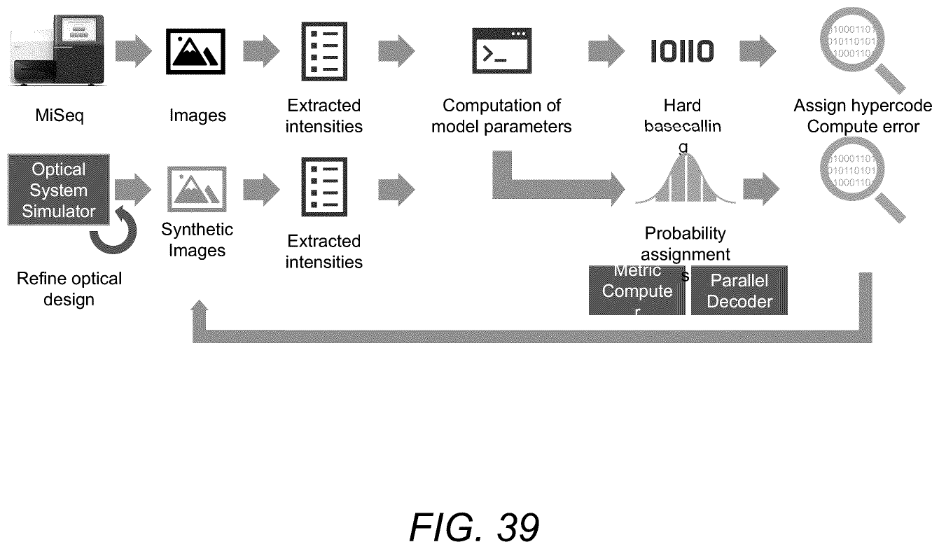

FIG. 39 is a schematic diagram illustrating an overview of an encoded assay analysis process.

In the methods of the invention, each code may include at least one segment encoding one or more symbols that correspond to a sequence of one or more nucleotides.

In some instances, the codes that are amplified are decoded using a soft decision decoding method. For example, decoding the codes that are amplified may include recording signal produced in response to interrogation of each segment of the codes and, upon completion of the interrogation, determining a probably of the presence of each of the codes by applying a soft-decision probabilistic decoding algorithm to the recorded signal. The signal produced may include, but is not limited to, signal from one or a combination of nanopore sequencing, next-generation sequencing, massively parallel sequencing, Sanger sequencing, sequencing by synthesis (SBS), pyrosequencing, sequencing by hybridization, decoding by hybridization, single molecule real-time sequencing, SOLID, and sequencing by ligation.

For the codes, each segment may comprise one symbol corresponding to one nucleotide. In one instance, each of the codes includes up to 50 segments for a length of each code up to 50 nucleotides. In this instance, decoding the codes that are amplified may include using sequencing by synthesis (SBS).

In other instances, each segment includes one symbol corresponding to more than one nucleotide.

In various embodiments, each code may include two or more segments, three or more segments, four or more segments, or five to sixteen segments.

In one instance, interrogation of the segments includes decoding by hybridization. At least one of the segments may be interrogated more than one time by hybridization with one or more hybridization probes each having at least one label to produce the signal. In some cases, at least four different labels may be utilized in the decoding by hybridization. The label may be an optical label or a fluorescent label.

In one example, each code includes at least four segments and at least sixteen symbols.

In the methods of the invention, the unique number of possibilities at each of the segments may include up to the number of different labels raised to the power of the number of the hybridizations per segment.

In one embodiment, at least one hybridization probe has two or more of the labels to create a pseudo label and generate a larger number of the symbols.

In the methods described herein, the set of targets may include tens of target analytes, hundreds of target analytes, thousands of target analytes, or tens of thousands of target analytes. In some embodiments, a method is provided of conducting an assay for a set of targets, the method comprising: (a) dividing a sample comprising a set of target nucleic acid sequences into two parallel samples for analysis, one for methylation specific analysis (MS sample) and one for non-methylation specific analysis (nMS sample); (b) adding a methylation specific binding moiety to the MS sample and incubating for a period of time sufficient for binding to methylated cytosines; (c) hybridizing an encoded detection probe in a set of encoded detection probes to each target in the set, each of the probes in the set comprising a target-specific binding site and a nucleotide sequence code from a set of nucleotide sequence codes, each code comprising at least one segment encoding one or more symbols that correspond to a sequence of one or more nucleotides, to yield a set of coded targets comprising the target and the encoded detection probe; (d) performing a molecular transformation on the set of coded targets in which a set of modified probes comprising the codes is produced to enable differentiation of a methylated and an unmethylated status of each target in the MS sample; and (e) amplifying and detecting the codes of the set of modified probes bound to target in the nMS sample and bound to the targets that are unmethylated in the MS sample, wherein the targets are detected by decoding the amplified codes associated with the targets.

In other instances, a method is provided of conducting an assay for a set of targets, the method comprising: (a) performing a hybridization and ligation reaction on a sample comprising a set of target nucleic acid sequences in which each target is hybridized to an encoded detection probe in a set of encoded detection probes, each of the probes in the set comprising: (i) a nucleotide sequence code from a set of nucleotide sequence codes comprising at least one segment encoding one or more symbols that correspond to a sequence of one or more nucleotides, and (ii) a sequence for a methylation sensitive endonuclease site, to yield a set of coded targets comprising the target and the encoded detection probe wherein the bound probes are circularized; (b) performing a transformation process on the set of coded targets, in which the methylation sensitive endonuclease cuts the targets that are unmethylated and does not cut the methylated targets to produce a set of modified encoded detection probes; and (c) performing an amplification and detection process on the set of modified encoded detection probes, in which the circularized probes bound to methylated targets are amplified and the cut probes bound to unmethylated targets are not amplified, wherein the methylated targets are detected by decoding the amplified codes associated with the methylated targets.

In various embodiments, a method is provided of conducting an assay for a set of targets, the method comprising: (a) performing a transformation process on a sample comprising a set of target nucleic acid sequences, in which a methylation sensitive endonuclease cuts the targets that are unmethylated at a target site but does not cut the methylated targets; (b) performing a hybridization and ligation reaction with an encoded detection probe from a set of encoded detection probes unique for each of the targets, each of the probes in the set comprising: (i) a nucleotide sequence code from a set of nucleotide sequence codes, each code comprising at least one segment encoding one or more symbols that correspond to a sequence of one or more nucleotides and (ii) a sequence for the methylation sensitive endonuclease site, wherein the probes hybridized to methylated targets are circularized thereby generating a set of modified encoded detection probes that are amplifiable, and wherein the probes unique for unmethylated targets are not circularized and are not amplifiable; and (c) performing an amplification and detection process on the set of modified encoded detection probes, wherein the methylated targets are detected by decoding the amplified codes associated with the methylated targets.

In some instances, a method is provided of conducting an assay for a set of targets, the method comprising: (a) performing a transformation process on a sample comprising a set of target nucleic acid sequences, in which a methylation sensitive endonuclease cuts the targets that are unmethylated at a target site but does not cut the methylated targets; (b) performing a hybridization and ligation reaction with an encoded detection probe from a set of encoded detection probes unique for each of the targets, each of the probes in the set comprising: (i) a nucleotide sequence code from a set of nucleotide sequence codes, each code comprising at least one segment encoding one or more symbols that correspond to a sequence of one or more nucleotides and (ii) a sequence for the methylation sensitive endonuclease site, wherein the probes hybridized to methylated targets are circularized thereby generating a set of modified encoded detection probes that are amplifiable, and wherein the probes unique for unmethylated targets are not circularized and are not amplifiable; and (c) performing an amplification and detection process on the set of modified encoded detection probes, wherein the methylated targets are detected by decoding the amplified codes associated with the methylated targets.

In other embodiments, a method is provided of conducting an assay for a set of targets, the method comprising: (a) performing a hybridization and ligation reaction on a sample comprising a set of target nucleic acid sequences in which a pair of encoded ligation detection probes, from a set of pairs of encoded ligation detection probes is hybridized to each of the targets, each pair of probes in the set of encoded ligation detection probes comprising: (i) a nucleotide sequence code from a set of nucleotide sequence codes, each code comprising at least one segment encoding one or more symbols that correspond to a sequence of one or more nucleotides, and (ii) a sequence for a methylation sensitive endonuclease, wherein the pair of probes are hybridized to the target immediately adjacent to each other leaving no gap and are ligated to produce a ligated dual probe strand, to yield a set of coded targets comprising the target and the encoded ligation detection probe; (b) performing a methylation sensitive endonuclease reaction on the on the set of coded targets in which the hybridized targets that are unmethylated are cut and the hybridized targets that are methylated are not cut, and performing a single-stranded DNA ligation reaction in which uncut methylated target is circularized to generate an intact primer site and in which cut unmethylated target is circularized to generate two circular elements that do not include an intact primer site to produce a set of modified detection probes; and (c) performing an amplification and detection process on the set of modified detection probes, wherein the methylated targets are detected by decoding the amplified codes associated with the methylated targets. The methods provided also include instances where steps (a) and (b) are reversed.

In another example, a method is provided of conducting an assay for a set of targets, the method comprising: (a) performing a hybridization and methyl binding moiety reaction on a sample comprising a set of target nucleic acid sequences, in which: (i) a location-specific oligonucleotide probe is hybridized adjacent to a site on each of the targets, the location-specific oligonucleotide probe comprising a first hybridization sequence complementary to the target and a second hybridization sequence complementary to a corresponding sequence on a linear encoded detection probe in a set of linear encoded detection probes, (ii) a methylation specific binding moiety is bound to a methyl group on each of the targets that are methylated at the site, the moiety modified with an oligonucleotide complementary to a corresponding sequence on the detection probe, and (iii) the detection probe is hybridized to the sequence on the location specific oligonucleotide probe and the oligonucleotide on the methyl binding moiety which effectively circularizes the detection probe, each of the detection probes in the set comprising a nucleotide sequence code from a set of nucleotide sequence codes, each code comprising at least one segment encoding one or more symbols that correspond to a sequence of one or more nucleotides to yield a set of coded targets comprising target and the encoded detection probe; (b) performing a ligation reaction on the set of coded targets, in which the detection probe is circularized to generate an intact primer site for the targets that are methylated at the site, but not for the targets that are not methylated at the site to produce a set of modified detection probes; and (c) performing an amplification and detection process on the set of modified detection probes, wherein the methylated targets are detected by decoding the amplified codes associated with the methylated targets.

In various embodiments, a method is provided of conducting an assay for a set of targets, the method comprising: (a) contacting a sample comprising a set of target nucleic acid sequences with a substrate having a methylation specific binding moiety immobilized thereto, wherein targets having one or more methylated cytosines are captured on the substrate; (b) hybridizing a set of target-specific, encoded detection probes to the fully or partially methylated targets in the set, the probes comprising: (i) a nucleotide sequence code from a set of nucleotide sequence codes, each code comprising at least one segment encoding one or more symbols that correspond to a sequence of one or more nucleotides and (ii) an amplification primer binding site, to yield a set of coded targets comprising the target and the encoded detection probe; (c) optionally, performing a ligation reaction in which the detection probe is circularized; and (d) performing an amplification and detection process on the set of coded targets, wherein the methylated targets are detected by decoding the amplified codes associated with the methylated targets.

In the methods of the invention, the set of encoded detection probes may comprise at least 10, 100, 1,000, or 10,000 encoded detection probes and each of the encoded detection probes may comprise a soft decodable code.

Decoding the amplified codes in the methods of the invention may include: (a) recording signal produced in response to interrogation of each segment of the codes; and (b) upon completion of the interrogation, determining a probably of the presence of each of the codes by applying a soft-decision probabilistic decoding algorithm to the recorded signal, wherein the presence of the code is indicative of the presence of the target. In some embodiments, interrogation of the segments may include one or a combination of nanopore sequencing, next-generation sequencing, massively parallel sequencing, Sanger sequencing, sequencing by synthesis (SBS), pyrosequencing, sequencing by hybridization, decoding by hybridization, single molecule real-time sequencing, SOLID, and sequencing by ligation.

### Examples

The invention provides a method for using dual hybridization probes in combination with a methylation sensitive endonuclease digestion reaction to differentiate a methylated target sequence and an unmethylated target sequence as described above with reference to FIG. 9. To demonstrate the feasibility of using a methylation sensitive endonuclease to differentiate a methylated target sequence and an unmethylated target sequence, the cutting event was evaluated using a qPCR assay. Briefly, a methylated target sample and an unmethylated target sample were digested with the methylation sensitive endonuclease Hha I for 30 minutes or 2 hours and then amplified in qPCR assay to determine the amount of amplifiable target present in each sample. In this assay, a methylated target should remain intact and amplifiable, while an unmethylated target should be cut and have less efficient amplification.

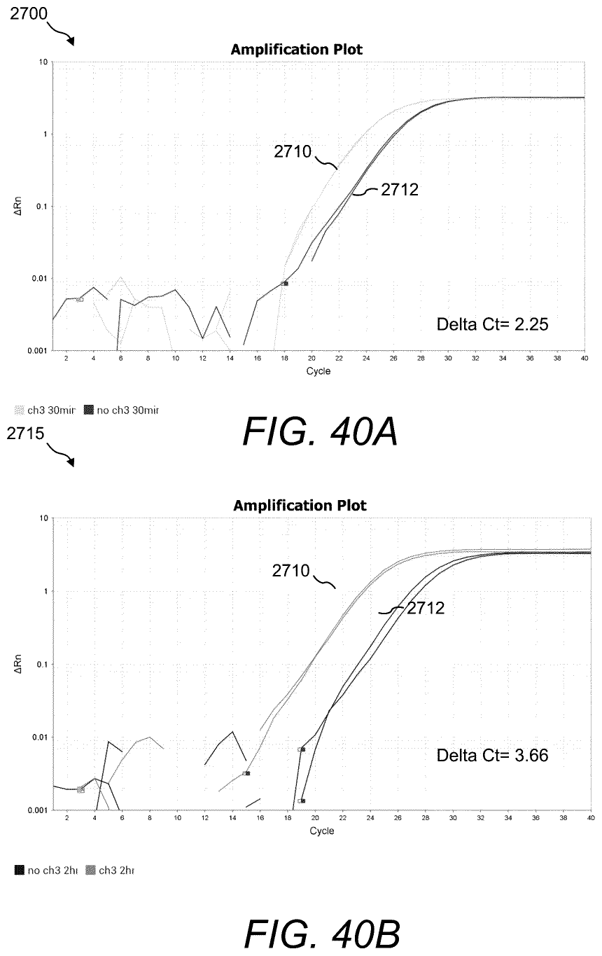

FIG. 40A and FIG. 40B are a pair of plots 2700 and 2715 showing qPCR amplification plots after 30 minutes and 2 hours, respectively, of cutting with Hha I. The data show that at a lower cycle value, the methylated target plot line 2710 had more material that was amplifiable than the unmethylated target plot line 2712, indicating that the unmethylated target was cleaved.

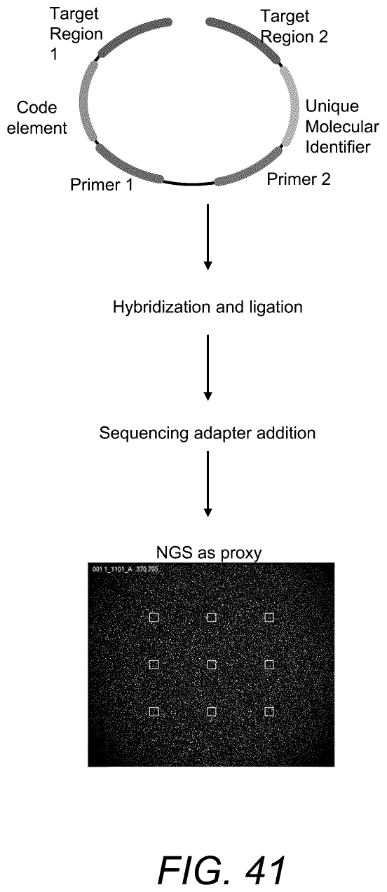

To demonstrate the feasibility of using a panel of code element probes (e.g., coded padlock probes) to detect different targets in a multiplexed hybridization/ligation assay, we used 12 different targets with 3 targets for testing for allele specificity and 14 probes with unique code elements were designed (some probes targeting multiple regions). As an assay read out, the steps of hybridization and ligation were performed followed by a step to introduce sequencing adapters to generate a library for next generation sequencing (NGS). Cluster codes and UMIs enabled counting of ligation events. FIG. 41 is a schematic diagram showing an example of a code element probe and some of the workflow steps in the multiplexed assay.

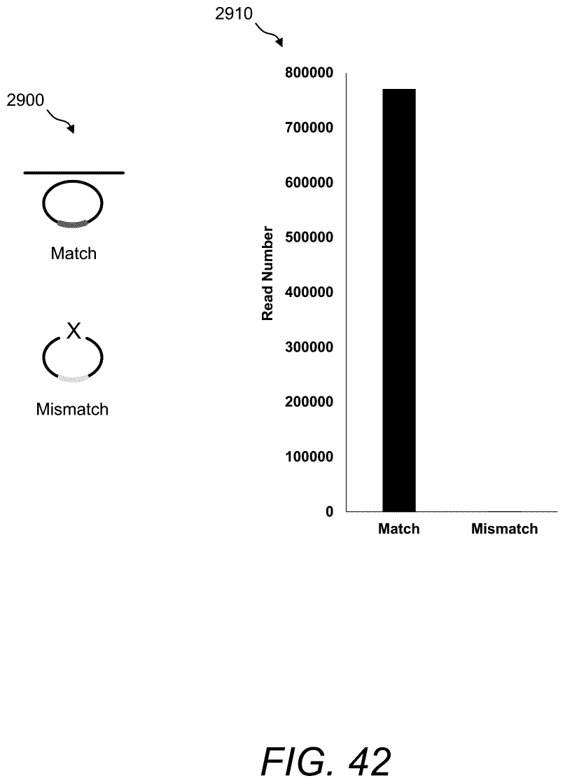

FIG. 42 is a schematic diagram 2900 and a plot 2910 showing the specificity of target interaction in the multiplexed assay. Schematic diagram 2900 shows a match and a mismatch between a probe and target sequence, where a match between a probe and a target results in a closing event, and a mismatch (X) does not. The dark gray arc and the light gray arc in the example probes represent different code elements. Plot 2910 shows the count of read number for the match and mismatch events. The data shows that the multiplexed assay provides high specificity of target interaction, i.e., about a 1000 to 1 read counts for matched vs mismatched.

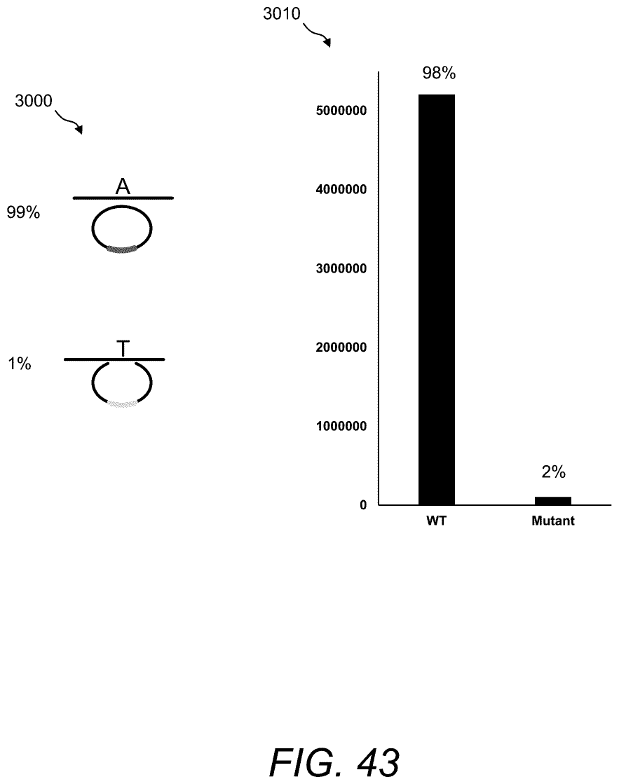

FIG. 43 is a schematic diagram 3000 and a plot 3010 showing the allele ratio in the multiplexed assay. Schematic diagram 3000 shows a match between a probe and a wildtype allele (A), and a mismatch between a probe and mutant allele (T), input into the assay at a ratio of 99% and 1%. The dark gray arc and the light gray arc in the example probes represent different code elements. Plot 3010 shows the read counts for the wildtype (WT) and mutant alleles. The data shows that the read count is close to the expected ratio for wildtype (A) and mutant alleles (T), at 98% and 2%, respectively.

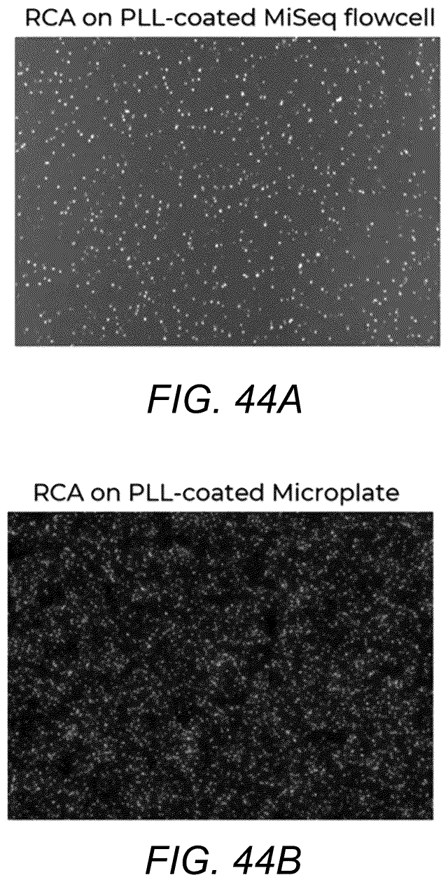

In another experiment, nanoballs were generated in an RCA reaction performed on a polylysine coated surface. Specifically, FIG. 44A and FIG. 44B are photos showing the density, size and uniformity of nanoballs generated in an RCA reaction performed on a polylysine-coated MiSeq flow cell or on a polylysine-coated microplate, respectively. In this example, RCA was performed as follows: RCA on Polylysine surface: MiSeq flowcells were washed to remove surface coatings before 0.01% poly-lysine (PLL) was applied, incubated for 30 minutes, washed and dried. PLL-coated microplates are assembled using purchased PLL-coated glass coverslips and plastic multi-well chambers. RCA reactions are prepared normally in tubes on ice containing phi29 polymerase, buffer, a primer and ligated purified probes, and the complete reaction is applied to the flowcell or microplate. The flowcell or microplate was incubated at 30 C. for 6-8 hours, and then washed with Tris/EDTA to stop the reaction. NBs were detected with different methods. The NBs on the MiSeq flowcell were detected by SBS using a MiSeq instrument while the NBs on the microplate surface were hybridized with a fluorophore-labeled oligonucleotide probe and imaged on a Lionheart automated microscope.

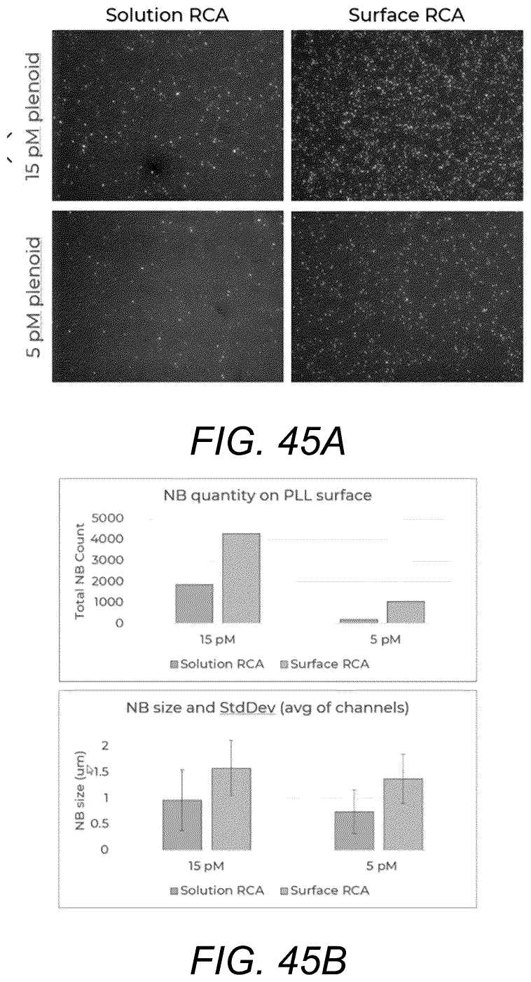

FIG. 45A and 45B are panel of photos and a pair of plots, respectively, of a comparison of nanoballs generated on a polylysine (PLL) surface to nanoballs absorbed to a surface after an RCA solution reaction. In this example, surface vs solution RCA reactions were performed as follows: RCA reactions were prepared normally in tubes on ice containing phi29 polymerase, buffer, a primer and either 5 pM or 15 pM ligated purified probes. A fraction of the RCA reactions was applied to different wells of a microplate with a PLL-coated bottom surface, and then the plate was incubated at 30 C. for 4 hours. The remainder of the RCA reactions in tubes were placed at 30 C. for 4 hours. The RCA reactions in the microplate were stopped by washing with Tris/EDTA. EDTA and TBS were added to the RCA reactions in tubes and fluorophore-labeled oligonucleotide probes were also added before the reactions were applied to the PLL-coated microplate and allowed to absorb for 1 hr. Fluorophore-labeled oligonucleotide probes in TBS were also applied to the wells in which the RCA was performed in the microplate for specific detection of NBs. After washing, all wells were imaged on a Lionheart automated microscope and analyzed with Lionheart software.

Various modifications and variations of the disclosed methods, compositions and uses of the invention will be apparent to the skilled person without departing from the scope and spirit of the invention. Although the invention has been disclosed in connection with specific preferred aspects or embodiments, it should be understood that the invention as claimed should not be unduly limited to such specific aspects or embodiments.

The present invention may be implemented using hardware, software, or a combination thereof and may be implemented in one or more computer systems or other processing systems. In one aspect, the invention is directed toward one or more computer systems capable of carrying out the functionality described herein.

In one embodiment, the system includes (a) a reaction vessel; (b) a reagent dispensing module; and (c) software to execute the method of any of the foregoing claims, wherein the method is executed robotically.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

We claim:

1. A method for conducting an assay for a set of target nucleic acid sequences, the method comprising:
a) dividing a sample comprising a set of target nucleic acid sequences into subsamples for analysis, yielding a set of subsamples comprising a methylation specific analysis subsample and a non-methylation specific analysis subsample, each comprising a subset of the target nucleic acid sequences;
b) adding a methylation specific binding moiety to the methylation specific analysis subsample and incubating for a time sufficient for binding the methylation specific binding moiety to methylated cytosines;
c) hybridizing an encoded detection probe from a set of encoded detection probes to each target nucleic acid sequence in the set of target nucleic acid sequences of the set of subsamples, each encoded detection probe in the set of encoded detection probes comprising a target specific binding site and a nucleic acid sequence code from a set of nucleic acid sequence codes, each nucleic acid sequence code comprising at least one segment encoding one or more symbols that corresponds to a sequence of one or more nucleotides, to yield a set of coded targets comprising a target nucleic acid sequence of the set of target nucleic acid sequences and the encoded detection probe;
d) performing a molecular transformation on the set of coded targets in which a set of modified probes comprising the set of nucleic acid sequence codes is produced to enable differentiation of a methylated status and an unmethylated status of each target nucleic acid sequence in the methylation specific analysis sample; and
e) amplifying and detecting the set of nucleic acid sequence codes of the set of modified probes in the non-methylation specific analysis sample, wherein the target nucleic acid sequences are detected by decoding the amplified codes associated therewith.

2. The method of claim 1, further comprising determining a fraction or percent of methylation at one or more of the target nucleic acid sequences in the set of target nucleic acid sequences of the set of subsamples using the non-methylation specific analysis subsample as a baseline reference.

3. The method of claim 1, wherein:
a) the molecular transformation comprises circularizing the encoded detection probe in the non-methylation specific analysis subsample, only if a target nucleic acid sequence matching the encoded detection probe sequence is present; and
b) the molecular transformation comprises circularizing the encoded detection probe in the methylation specific analysis subsample, only if an unmethylated target nucleic acid sequence matching the encoded detection probe sequence is present.

4. The method of claim 1, wherein decoding the set of nucleic acid sequence codes that are amplified comprises:
a) recording a signal produced in response to an interrogation of each segment of each code of the set of nucleic acid sequence codes; and
b) upon completion of the interrogation, determining a probability of the presence of each of the nucleic acid sequence codes by applying a soft decision probabilistic decoding algorithm to the recorded signal, wherein the presence of the nucleic acid sequence code is indicative of the presence of that target nucleic acid sequence.

5. The method of claim 1, wherein decoding the set of nucleic acid sequence codes that are amplified comprises one or a combination of nanopore sequencing, next-generation sequencing, massively parallel sequencing, Sanger sequencing, sequencing by synthesis, pyrosequencing, sequencing by hybridization, decoding by hybridization, single molecule real-time sequencing, sequencing by ligation, microarray detection, oligonucleotide probes in a hybridization based reaction, electronic or electrical sensing mechanism, and in situ sequencing.

6. The method of claim 1, wherein the encoded detection probe comprises one or a combination of sequencing primers, one or more amplification primer sequences, unique identifiers sequences or sample indexes.

7. The method of claim 6, wherein the one or more amplification primer sequences comprise a universal primer sequence that is common to each encoded detection probe in the set of encoded detection probes.

8. The method of claim 1, wherein the amplifying comprises rolling circle amplification or PCR amplification.

9. The method of claim 1, further comprises performing an exonuclease reaction to digest single stranded nucleic acid that is present after the amplifying.

10. The method of claim 1, wherein the methylation specific binding moiety comprises one or a combination of monoclonal antibodies, polyclonal antibodies, nanobodies, methyl CpG binding domain, Uhrf1pr proteins, or methylation-specific aptamers.

11. The method of claim 1, wherein the sample and set of target nucleic acid sequences comprises one or more of:
a) wild type nucleic acid sequences,
b) mutant nucleic acid sequences,
c) double stranded DNA targets,
d) a biopsy sample,
e) cell free DNA,
f) biological markers for screening or diagnosing cancer,
g) a panel of methylation markers for diagnosing cancer, and
h) a biological liquid or a biological tissue.

12. The method of claim 1, wherein each nucleic acid sequence code from the set of nucleotide sequence codes is a predetermined code and is one or more of:
a) selected to avoid interaction with other assay components,
b) selected to ensure that the nucleic acid sequence code differs from each other nucleic acid sequence code, and
c) is homopolymer free.

13. The method of claim 1, wherein each nucleic acid sequence code from the set of nucleotide sequence codes comprises two or more segments.

14. The method of claim 4, wherein the interrogation of each segment of each code of the set of nucleic acid sequence codes comprises decoding by hybridization and at least one of the segments is interrogated more than one time by hybridization with one or more hybridization probes each having at least one label to produce the signal, wherein at least four different labels are utilized in the decoding by hybridization, and optionally wherein each code comprises at least four segments and at least sixteen symbols.

15. A method of conducting an assay for a set of targets, the method comprising:
a) performing a hybridization and methyl binding moiety reaction on a sample comprising a set of target nucleic acid sequences, in which:
i) a location-specific oligonucleotide probe is hybridized adjacent to a site on each of the targets, the location-specific oligonucleotide probe comprising a first hybridization sequence complementary to the target and a second hybridization sequence complementary to a corresponding sequence on a linear encoded detection probe in a set of linear encoded detection probes,
ii) a methylation specific binding moiety is bound to a methyl group on each of the targets that are methylated at the site, the moiety modified with an oligonucleotide complementary to a corresponding sequence on the detection probe, and
iii) the detection probe is hybridized to the sequence on the location specific oligonucleotide probe and the oligonucleotide on the methyl binding moiety thereby circularizing the detection probe, each of the detection probes in the set comprising a nucleotide sequence code from a set of nucleotide sequence codes, each code comprising at least one segment encoding one or more symbols that correspond to a sequence of one or more nucleotides to yield a set of coded targets comprising the target, and the encoded detection probe;
b) performing a ligation reaction on the set of coded targets, in which the detection probe is circularized to generate an intact primer site for the targets, in which the detection probe is circularized to generate an intact primer site for the targets that are methylated at the site, but not for the targets that are not methylated at the site to produce a set of modified detection probes; and
c) performing an amplification and detection process on the set of modified detection probes, wherein the methylated targets are detected by decoding the amplified codes associated with the methylated targets.

16. A method of conducting an assay for a set of targets, the method comprising:
a) contacting a sample comprising a set of target nucleic acid sequences with a substrate having a methylation specific binding moiety immobilized thereto, wherein targets having one or more methylated cytosines are captured on the substrate;
b) hybridizing a set of target specific, encoded detection probes to the fully or partially methylated targets in the set, each of the probes comprising:
(i) a nucleotide sequence code from a set of nucleotide sequence codes, each code comprising at least one segment encoding one or more symbols that correspond to a sequence of one or more nucleotides; and
(ii) an amplification primer binding site to yield a set of coded targets comprising the target and the encoded detection probe;
c) performing a ligation reaction in which the detection probe is circularized; and
d) performing an amplification and detection process on the set of coded targets, wherein the methylated targets are detected by decoding the amplified codes associated with the methylated targets.

17. The method of claim 16, wherein the encoded detection probe is a linear encoded detection probe and the ligation reaction is performed to circularize the probe.

18. The method of claim 16, wherein the encoded detection probe is a pre-circularized encoded detection probe.

19. The method of claim 16, wherein the solid substrate is a well of a multi-well plate.

20. A system for conducting an assay for a set of target nucleic acid sequences, comprising:
a) a reaction vessel;
b) a reagent dispensing module; and
c) software to execute the method of claim 1, wherein the method is executed robotically.
